(19) United States
(12) Patent Application Publication
(10) Pub. No.: US 2015/0299724 A1
(43) Pub. Date: Oct. 22, 2015

(54) Transgenic Plants Exhibiting Improved Resistance to Abiotic Stress

(71) Applicant: A.B. Seeds Ltd., Lod (IL)

(72) Inventors: Rudy Maor, Rechovot (IL); Iris Nesher, Tel-Aviv (IL)

(73) Assignee: A. B. Seeds Ltd., Lod (IL)

(21) Appl. No.: 14/438,748

(22) PCT Filed: Oct. 28, 2013
(86) PCT No.: PCT/IL13/50882

§ 371 Date: Apr. 27, 2015

Related U.S. Application Data
(60) Provisional application No. 61/719,413, filed on Oct. 28, 2012.

(51) Int. Cl. C12N 15/82

(52) U.S. Cl. CPC C12N 15/8271; C12N 15/8273; C12N 15/8274; C12N 15/8275; C12N 15/8277; C12N 15/8278; C12N 15/8218

(57) ABSTRACT

This application provides and discloses small RNAs and their target genes that are involved in response and resistance to abiotic stresses, and methods of modulating expression or activity of these small RNAs and target genes. This application further provides transgenic plants, plant parts, e.g., seeds, that have altered expression or activity of these small RNAs and target genes and have improved abiotic stress tolerance. This application also provides methods of producing and growing transgenic plants or seeds that have improved abiotic stress tolerance. In specific embodiments, this application discloses small RNAs, small RNA target genes, and uses thereof to improve plant abiotic stress tolerance. In specific embodiments, this application also discloses mi RNA, mi RNA target genes, and uses thereof to improve plant drought tolerance.

### CROSS-REFERENCE TO RELATED APPLICATIONS

This application claims priority to U.S. Provisional Application No. 61/719,413 filed Oct. 28, 2012, which is herein incorporated by reference in its entirety.

### INCORPORATION OF SEQUENCE LISTING

This application contains an electronic equivalent paper copy of the sequence listing submitted herewith electronically via EFS web and a computer-readable form of the sequence listing submitted herewith electronically via EFS web and contains the file named “57696.txt” and is hereby incorporated by reference in its entirety.

### FIELD OF THE DISCLOSURE

Methods and compositions for improving plant tolerance to abiotic stresses are provided. Also provided are miRNAs and their target genes, and transgenic and non-transgenic uses thereof for improving plant drought tolerance.

### BACKGROUND

Consumption of soybean for food production is increasing worldwide because of its reported beneficial health effects (Friedman and Brandon, 2001, J. Agric. Food Chem., 49:1069-1086). Soybean is also viewed as an attractive crop for the production of biodiesel (reviewed in Manavalan et al., 2009, Plant Cell Physiol., 50(7):1260-76). Importantly, it has the ability to fix atmospheric nitrogen, which in turn may cut the input of nitrogen fertilizer that often accounts for the single largest energy input in agriculture.

With a growing world population, increasing demand for food, fuel and fiber, and a changing climate, agriculture faces unprecedented challenges. In general, shortage in water supply is one of the most severe global agricultural problems affecting plant growth and crop yield. Excessive efforts are made to alleviate the harmful effects of desertification of the world's arable land. Farmers are seeking advanced, biotechnology-based solutions to enable them to obtain stable high yields and give them the potential to reduce irrigation costs or to grow crops in areas where potable water is a limiting factor. It should be noted that improved abiotic stress tolerance will confer plants with improved vigor also under non-stress conditions, resulting in crops having improved biomass and/or yield.

Identification of stress response genes and their expression in transgenic plants has been extensively undertaken. However, the expression of stress response genes introduced into plants is commonly suboptimal. Reasons for the poor expression may include inappropriate choice of promoters and/or other regulatory elements and destruction of exon-intron structure. In contrast to the abundance of genes involved in the responses to abiotic stress in plants, there is limited information on small RNA molecules involved in plant response and adaptation to abiotic stress.

### SUMMARY

This application provides and discloses small RNAs and their target genes that are involved in response and resistance to abiotic stresses, and methods of modulating expression or activity of these small RNAs and target genes. This application further provides transgenic plants, plant parts, e.g., seeds, that have altered expression of these small RNAs and target genes and have improved abiotic stress tolerance. This application also provides methods of producing and growing transgenic plants or seeds that have improved abiotic stress tolerance. In specific embodiments, this application discloses small RNAs, small RNA target genes, and uses thereof to improve plant drought tolerance.

In one aspect, the instant application discloses a method of improving abiotic stress tolerance in a soybean plant comprising transgenically expressing in the soybean plant a recombinant DNA construct comprising a heterologous promoter operably linked to at least one DNA selected from the group consisting of: a DNA encoding at least one miRNA precursor that yields a mature miRNA selected from the group consisting of a mature miR164 and a mature miR168; a DNA encoding a miR397 target mimic, a miR408 target mimic, or a miR1093 target mimic; and a DNA encoding a miR397-, miR408-, or miR1093-resistant target gene, wherein the miR397-, miR408-, or miR1093-resistant target gene comprises an introduced silent mutation in a nucleotide sequence that is otherwise substantially identical to the nucleotide sequence of an endogenous gene that is natively regulated by miR397, miR408, or miR1093, and wherein the silent mutation prevents binding by a mature miR397, miR408, or miR1093 to a transcript of the miR397-, miR408-, or miR1093-resistant target gene.

In another aspect, the instant application discloses a method of providing a plant with increased root branching or root depth comprising transgenically expressing in the plant a recombinant DNA construct comprising a heterologous promoter operably linked to at least one DNA selected from the group consisting of: a DNA encoding at least one miRNA precursor that yields a mature miRNA selected from the group consisting of a mature miR164 and a mature miR168; a DNA encoding a miR397 target mimic, a miR408 target mimic, or a miR1093 target mimic; and a DNA encoding a miR397-, miR408-, or miR1093-resistant target gene, wherein the miR397-, miR408-, or miR1093-resistant target gene comprises an introduced silent mutation in a nucleotide sequence that is otherwise substantially identical to the nucleotide sequence of an endogenous gene that is natively regulated by miR397, miR408, or miR1093, and wherein the silent mutation prevents binding by a mature miR397, miR408, or miR1093 to a transcript of the miR397-, miR408-, or miR1093-resistant target gene.

In one aspect, the instant application discloses a method of producing a transgenic soybean plant, the method comprising transforming a soybean plant cell with a transgene comprising a heterologous promoter operably linked to at least one DNA encoding a mature miRNA, comprising a sequence having at least 95% sequence identity to a sequence selected from the group consisting of SEQ ID NOs: 1, 2, and 11-94, and producing a transgenic soybean plant from the transformed cell, wherein the transgenic soybean plant has improved drought tolerance compared to a control soybean plant lacking the transgene.

In another aspect, the instant application discloses a method of producing a transgenic soybean plant, the method comprising transforming a soybean plant cell with a transgene comprising a heterologous promoter operably linked to at least one DNA having at least 95% sequence identity to a sequence selected from the group consisting of SEQ ID NOs: 417-419 and 386-410, and producing a transgenic soybean plant from the transformed cell, wherein the transgenic soybean plant has improved drought tolerance compared to a control soybean plant lacking the transgene.

In one aspect, the instant application discloses a transgenic soybean plant, or part thereof, comprising a transgene that encodes a mature miRNA comprising a sequence having at least 95% sequence identity to a sequence selected from the group consisting of SEQ ID NOs: 1, 2, and 11-94, wherein the transgenic soybean plant has improved drought tolerance compared to a non-transgenic control soybean plant.

In another aspect, the instant application discloses a transgenic soybean plant, or part thereof, comprising a transgene that encodes a miR397 target mimic, a miR408 target mimic, or a miR1093 target mimic, and the transgenic soybean plant has improved drought tolerance compared to a non-transgenic control soybean plant.

In a further aspect, the instant application discloses a transgenic soybean plant, or part thereof, comprising a transgene that encodes a miR397-, miR408-, or miR1093-resistant target gene, wherein the miR397-, miR408-, or miR1093-resistant target gene comprises a sequence having at least 90% sequence identity to a sequence selected from the group consisting of SEQ ID NOs: 386-410, and the transgenic soybean plant has improved drought tolerance compared to a non-transgenic control soybean plant.

In one aspect, the instant application discloses a method of improving abiotic stress tolerance in a soybean plant comprising transgenically expressing in the soybean plant a transgene comprising a sequence having at least 80%, 85%, 88%, 90%, 92%, 95%, 96%, 97%, 98%, or 99% sequence identity to a sequence selected from the group consisting of SEQ ID NOs: 1 to 262, 317 to 370, and 380 to 419, wherein the transgenic plant has improved drought tolerance compared to a control plant lacking the transgene.

In one aspect, the instant application discloses a method of improving abiotic stress tolerance in a soybean plant comprising transgenically expressing in the soybean plant a transgene encoding an amino acid sequence having at least 80%, 85%, 88%, 90%, 92%, 95%, 96%, 97%, 98%, or 99% sequence identity to a sequence selected from the group consisting of SEQ ID NOs: 263 to 317 and 371 to 379, wherein the transgenic plant has improved drought tolerance compared to a control plant lacking the transgene.

In one aspect, the instant application discloses a method of improving abiotic stress tolerance in a soybean plant comprising transgenically expressing in the soybean plant a transgene encoding a small RNA or in a particular aspect a miRNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, and producing a transgenic plant from the transformed cell, wherein the transgenic plant has improved drought tolerance compared to a control plant lacking the transgene. In a further aspect, a method of the instant application further comprises collecting a seed from the transgenic plant.

In one aspect, the instant application discloses a method of improving abiotic stress tolerance in a soybean plant comprising transgenically expressing in the soybean plant a transgene encoding a target nucleic acid molecule that is complementary to a small RNA or in a particular aspect a miRNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, and producing a transgenic plant from the transformed cell, wherein the transgenic plant has improved drought tolerance compared to a non-transgenic control plant.

In one aspect, a transgenic plant, or part thereof, disclosed herein comprises a transgene comprising a sequence having at least 80%, 85%, 88%, 90%, 92%, 95%, 96%, 97%, 98%, or 99% sequence identity to a sequence selected from the group consisting of SEQ ID NOs: 1 to 262, 317 to 370, and 380 to 419, wherein the transgenic plant has improved drought tolerance compared to a control plant lacking the transgene.

In another aspect, a transgenic plant, or part thereof, disclosed herein comprises a transgene encoding a polypeptide sequence having at least 80%, 85%, 88%, 90%, 92%, 95%, 96%, 97%, 98%, or 99% sequence identity to a sequence selected from the group consisting of SEQ ID NOs: 263 to 317 and 371 to 379, wherein the transgenic plant has improved drought tolerance compared to a control plant lacking the transgene.

In one aspect, a transgenic plant, or part thereof, disclosed herein comprises a transgene encoding a small RNA or in a particular aspect a miRNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, wherein the transgenic plant has improved drought tolerance compared to a control plant lacking the transgene.

In one aspect, a transgenic plant, or part thereof, disclosed herein comprises a transgene encoding a small RNA target nucleic acid molecule that is complementary to a small RNA or in a particular aspect a miRNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, wherein the transgenic plant has improved drought tolerance compared to a non-transgenic control plant.

### DETAILED DESCRIPTION

Unless defined otherwise, technical and scientific terms as used herein have the same meaning as commonly understood by one of ordinary skill in the art. One skilled in the art will recognize many methods can be used in the practice of the present disclosure. Indeed, the present disclosure is in no way limited to the methods and materials described. Any references cited herein are incorporated by reference in their entireties. For purposes of the present disclosure, the following terms are defined below.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to a mature gma-miR164 molecule, or the RNA sequence of a mature gma-miR164 molecule. Similarly, though SEQ ID NO: 6 is expressed in a RNA sequence format (e.g., reciting U for uracil), depending on the actual type of molecule being described, SEQ ID NO: 6 can refer to either the sequence of a RNA molecule comprising a stem loop structure giving rise to a gma-miR164 molecule, or the sequence of a DNA molecule that corresponds to the RNA sequence shown. In any event, both DNA and RNA molecules having the sequences disclosed with any substitutes are envisioned.

As used herein, “small RNA” refers to any RNA molecule that is about 15-30 nucleotides long, preferably 20-24 nucleotides long. A small RNA can be either double-stranded or single-stranded. Small RNA includes, without limitation, miRNA (microRNA), ta-siRNA (trans activating siRNA), siRNA, activating RNA (RNAa), nat-siRNA (natural anti-sense siRNA), hc-siRNA (heterochromatic siRNA), cis-acting siRNA, lmiRNA (long miRNA), lsiRNA (long siRNA) and easiRNA (epigenetically activated siRNA) and their respective precursors. Preferred sRNA molecules of the disclosure are microRNA molecules, ta-siRNA molecules and RNAa molecules and their respective precursors.

As used herein, the term “siRNA” (also referred to herein interchangeably as “small interfering RNA”), is a class of double-stranded RNA molecules, 20-25 nucleotides in length. Without being limited by any theory, a role of siRNA is its involvement in the RNA interference (RNAi) pathway, where it interferes with the expression of a specific gene.

As used herein, the term “microRNA” (also referred to herein interchangeably as “miRNA” or “miR”) refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule (i.e., target), wherein the other RNA molecule comprises a nucleotide sequence essentially complementary to the nucleotide sequence of the miRNA molecule. It is understood that the combination of “miR” with a number, e.g., miR164, refers to one or more microRNAs including, without limitation, family members.

While not limited by a particular theory, a miRNA molecule is often processed from a “pre-miRNA” or as used herein a precursor of a miRNA molecule by proteins, such as DCL proteins. Pre-microRNA molecules are typically processed from pri-microRNA molecules (primary transcripts). The single-stranded RNA segments flanking the pre-microRNA are important for processing of the pri-miRNA into the pre-miRNA. The cleavage site appears to be determined by the distance from the stem-ssRNA junction (Han et al., 2006, Cell, 125:887-901).

As used herein, a “pre-miRNA” molecule is an RNA molecule of about 100 to about 200 nucleotides, preferably about 100 to about 130 nucleotides, which can adopt a secondary structure comprising a double-stranded RNA stem and a single-stranded RNA loop (also referred to as “hairpin”) and further comprising the nucleotide sequence of the miRNA (and its complement sequence) in the double-stranded RNA stem. According to a specific embodiment, the miRNA and its complement are located about 10 to about 20 nucleotides from the free ends of the miRNA double-stranded RNA stem. The length and sequence of the single-stranded loop region are not critical and may vary considerably, e.g., between 30 and 50 nt (nucleotide) in length. The complementarity between the miRNA and its complement need not be perfect and about 1 to 3 bulges of unpaired nucleotides can often be tolerated. The secondary structure adopted by an RNA molecule can be predicted by computer algorithms conventional in the art such as mFOLD. The particular strand of the double-stranded RNA stem from the pre-miRNA which is released by DCL activity and loaded onto the RISC complex is determined by the degree of complementarity at the 5′ end, whereby the strand, which at its 5′ end is the least involved in hydrogen bonding between the nucleotides of the different strands of the cleaved dsRNA stem, is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule.

As used herein, the term “target mimic” refers to a miR-specific inhibitor possessing at least one microRNA binding site, mimicking the microRNA target. In some embodiments, a target mimic may possess at least one nucleotide sequence comprising 6 consecutive nucleotides complementary to positions 2-8 of a corresponding small RNA. In some embodiments, the target mimic is a RNA molecule comprising a small RNA including without limitation miRNA, binding site modified to render it resistant to small RNA induced cleavage. In some embodiments, a variation is introduced in the nucleotide of the target sequence complementary to the nucleotides 10 or 11 of the small RNA resulting in a mismatch.

As used herein, the term “stem-loop precursor” refers to stem-loop precursor RNA structure from which the miRNA can be processed. In the case of siRNA, the precursor is typically devoid of a stem-loop structure.

As used herein, an “artificial microRNA” (amiRNA) is a type of miRNA which is derived by replacing native miRNA duplexes from a natural miRNA precursor. Generally, an artificial miRNA is a non-naturally-existing miRNA molecule produced from a pre-miRNA molecule scaffold engineered by exchanging a miRNA sequence of a naturally-existing pre-miRNA molecule for a sequence of interest which corresponds to the sequence of an artificial miRNA.

As used herein, with respect to a nucleic acid sequence, nucleic acid molecule, or a gene, the term “natural” or “native” means that the respective sequence or molecule is present in a wild-type plant cell, that has not been genetically modified or manipulated by man. A small RNA molecule naturally targeting a target gene means a small RNA molecule present in a wild-type plant cell, the cell has not been genetically modified or manipulated by man which is targeting a target gene naturally occurring in the respective plant cell.

As used herein, a “hybrid plant” refers to a plant, or a part thereof, resulting from a cross between two parent plants, wherein one or more parents are genetically engineered plants of the disclosure (transgenic plant expressing an exogenous small RNA sequence or a precursor thereof). Such a cross can occur by, for example, sexual reproduction, or in vitro nuclear fusion.

As used herein, the term “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present disclosure may comprise a particular type of a plant cell or a plurality of different types of plant cells.

As used herein a “transgenic plant” means a plant whose genetic material has been altered from its naturally-occurring composition. Alternations to genetic materials include, without limitation, the stable integration of recombinant DNA into a plant's nuclear genome. A transgenic plant as used herein further includes stable integration of recombinant DNA into the plant's chloroplast. A transgenic plant includes, without limitation, a plant developed from an originally-transformed plant cell and progeny transgenic plants from later generations or crosses of a transformed plant.

As used herein, the term “recombinant DNA” means DNA which has been genetically engineered and constructed outside of a cell.

As used herein, a “DNA construct” means a recombinant DNA having one or more of a promoter, a transcription terminator, an enhancer or other transcriptional regulatory element, post-transcriptional regulatory sequences including, for example, polyadenlyation and splicing signals. A DNA construct according to the present disclosure may further include targeting signals, for example, sequences providing for homologous recombination with a target genome or sequences for intracellular target such as nuclear localization signals.

As used herein, the term “structural gene” means a DNA sequence that is transcribed into mRNA which is then translated into a sequence of amino acids characteristic of a specific polypeptide, or processed into one or more specific small RNA molecules of about 21 to 24 nucleotides long.

As used herein, the term “nucleotide sequence of interest” refers to any nucleotide sequence, the manipulation of which may be deemed desirable for any reason (e.g., confer improved qualities), by one of ordinary skill in the art.

As used herein, the term “expression” refers to the biosynthesis of a gene product. For example, in the case of a structural gene, expression involves transcription of the structural gene into mRNA and, optionally, the subsequent translation of mRNA into one or more polypeptides. In another example, expression may involve the transcription of a small RNA precursor and, optionally, the subsequent processing of the small RNA precursor to an miRNA, ta-siRNA, siRNA, activating RNA, nat-siRNA, hc-siRNA, cis-acting siRNA, lmiRNA, lsiRNA, easiRNA, or their respective intermediates.

As used herein, the term “heterologous” means not naturally occurring together.

As used herein, the terms “promoter,” “promoter element,” and “promoter sequence” refer to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

As used herein, the terms “operable linkage” and “operably linked” are to be understood as meaning, for example, the sequential arrangement of a regulatory element (e.g., a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of the nucleic acid sequence. The expression may result depending on the arrangement of the nucleic acid sequences in relation to sense or antisense RNA. To this end, direct linkage in the chemical sense is not necessarily required.

As used herein, the terms “transcription terminator” and “transcription terminator sequence” are intended to mean a sequence which leads to or initiates a stop of transcription of a nucleic acid sequence initiated from a promoter. Preferably, a transcription terminator sequences further comprises sequences which cause polyadenylation of the transcript.

As used herein, the term “transformation” refers to the introduction of genetic material (e.g., a transgene) into a cell. Transformation of a cell may be stable or transient. The term “transient transformation” or “transiently transformed” refers to the introduction of one or more transgenes into a cell in the absence of integration of the transgene into the host cell's genome. The term “transient transformant” refers to a cell which has transiently incorporated one or more transgenes.

In contrast, the terms “stable transformation” and “stably transformed” refer to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction of genomic DNA of the cell to amplify transgene sequences. The term “stable transformant” refers to a cell which has stably integrated one or more transgenes into the genomic DNA. Thus, a stable transformant is distinguished from a transient transformant in that, whereas genomic DNA from the stable transformant contains one or more transgenes, DNA from the transient transformant does not contain a transgene. In certain preferred embodiments, a stable transformant comprises one or more integrated transgenes that segregate together in a Mendelian fashion. Transformation also includes introduction of genetic material into plant cells in the form of plant viral vectors involving epichromosomal replication and gene expression which may exhibit variable properties with respect to meiotic stability. Stable transformation also includes introduction of genetic material into cells in the form of viral vectors involving epichromosomal replication and gene expression which may exhibit variable properties with respect to meiotic stability.

As used herein, the term “Agrobacterium” refers to a soil-borne, Gram-negative, rod-shaped phytopathogenic bacterium which causes crown gall. The term “Agrobacterium” includes, but is not limited to, the strains Agrobacterium tumefaciens (which typically causes crown gall in infected plants), and Agrobacterium rhizogenes (which causes hairy root disease in infected host plants).

As used herein, the term “heterozygous” means a genetic condition existing when two different alleles reside at a specific locus, but are positioned individually on corresponding pairs of homologous chromosomes in the cell of a diploid organism. Conversely, as used herein, the term “homozygous” means a genetic condition existing when two identical alleles reside at a specific locus, but are positioned individually on corresponding pairs of homologous chromosomes in the cell of a diploid organism.

As used herein, the terms “homology” and “identity” when used in relation to nucleic acids, describe the degree of similarity between two or more nucleotide sequences. The percentage of “sequence identity” between two sequences is determined by comparing two optimally aligned sequences over a comparison window, such that the portion of the sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. An alignment of two or more sequences may be performed using any suitable computer program. For example, a widely used and accepted computer program for performing sequence alignments is CLUSTALW v1.6 (Thompson, et al. Nucl. Acids Res., 22: 4673-4680, 1994).

As used herein, the terms “exogenous polynucleotide” and “exogenous nucleic acid molecule” relative to a plant refer to a heterologous nucleic acid sequence which is not naturally expressed within that plant. An exogenous nucleic acid molecule may be introduced into a plant in a stable or transient manner. An exogenous nucleic acid molecule may comprise a nucleic acid sequence which is identical or partially homologous to an endogenous nucleic acid sequence of the plant.

As used herein, a “control plant” means a plant that does not contain the recombinant DNA that expresses a biomolecule (e.g., protein, miRNA, small RNA-resistant target mRNA, target mimic) that imparts an enhanced trait. Control plants are generally from same species and of the same developmental stage which is grown under the same growth conditions as the transformed plant. A suitable control plant can be a non-transgenic plant of the parental line used to generate a transgenic plant, i.e., devoid of recombinant DNA. A suitable control plant may in some cases be a progeny of a hemizygous transgenic plant line that is does not contain the recombinant DNA, known as a negative segregant.

As used herein, the term “wild-type” means, with respect to an organism, polypeptide, or nucleic acid sequence, that the organism, polypeptide, or nucleic acid sequence is naturally occurring or available in at least one naturally-occurring organism which is not changed, mutated, or otherwise manipulated by man.

As used herein, an “enhanced trait” means a characteristic of a transgenic plant that includes, but is not limited to, an enhanced agronomic trait characterized by enhanced plant morphology, physiology, growth and development, yield, nutritional enhancement, disease or pest resistance, or environmental or chemical tolerance. In more specific aspects of this disclosure, an enhanced trait is selected from the group of enhanced traits consisting of enhanced water use efficiency, enhanced cold tolerance, increased yield, enhanced nitrogen use efficiency, enhanced seed protein, and enhanced seed oil. In an important aspect of the disclosure, the enhanced trait is enhanced yield, including, but not limited to, increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability, and high plant density.

As used herein, the term “abiotic stress” refers to any adverse effect on metabolism, growth, viability, and/or reproduction of a plant. Abiotic stress can be induced by any suboptimal environmental growth conditions such as, for example, water deficit or drought, flooding, freezing, low or high temperature, strong winds, heavy metal toxicity, anaerobiosis, high or low nutrient levels (e.g., nutrient deficiency), high or low salt levels (e.g., salinity), atmospheric pollution, high or low light intensities (e.g., insufficient light), or UV irradiation. Abiotic stress may be a short term effect (e.g., acute effect, e.g., lasting for about a week) or alternatively may be persistent (e.g., chronic effect, e.g., lasting, for example, 10 days or more). The present disclosure contemplates situations in which there is a single abiotic stress condition or alternatively situations in which two or more abiotic stresses occur.

As used herein, the term “abiotic stress tolerance” refers to the ability of a plant to endure an abiotic stress without exhibiting substantial physiological or physical damage (e.g., alteration in metabolism, growth, viability, and/or reproductivity of the plant).

As used herein the terms “biomass,” “biomass of a plant,” and “plant biomass” refer to the amount (e.g., measured in grams of air-dry tissue) of a tissue produced from the plant in a growing season. An increase in plant biomass can be in the whole plant or in parts thereof such as aboveground (e.g., harvestable) parts, vegetative biomass, roots, and/or seeds.

As used herein, the terms “vigor,” “vigor of a plant,” and “plant vigor” refer to the amount (e.g., measured by weight) of tissue produced by the plant in a given time. Increased vigor could determine or affect the plant yield or the yield per growing time or growing area. In addition, early vigor (e.g., seed and/or seedling) results in improved field stand.

As used herein, the terms “yield,” “yield of a plant,” and “plant yield” refer to the amount (e.g., as determined by weight or size) or quantity (e.g., numbers) of tissues or organs produced per plant or per growing season. Increased yield of a plant can affect the economic benefit one can obtain from the plant in a certain growing area and/or growing time.

As used herein, the terms “improving,” “improved,” “increasing,” and “increased” refer to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or greater increase in nitrogen use efficiency (NUE), in tolerance to abiotic stress, in yield, in biomass, or in vigor of a plant, as compared to a control plant.

As used herein, “a reduction” of the level of an agent such as a protein or mRNA means that the level is reduced relative to a cell or organism lacking a dsRNA molecule capable of reducing the agent.

As used herein, the term “at least a partial reduction” of the level of an agent such as a protein or mRNA means that the level is reduced at least 25% relative to a cell or organism lacking a dsRNA molecule capable of reducing the agent.

As used herein, “a substantial reduction” of the level of an agent such as a protein or mRNA means that the level is reduced relative to a cell or organism lacking a dsRNA molecule capable of reducing the agent, where the reduction of the level of the agent is at least 75%.

As used herein, “an effective elimination” of an agent such as a protein or mRNA is relative to a cell or organism lacking a dsRNA molecule capable of reducing the agent, where the reduction of the level of the agent is greater than 95%. An agent, preferably a dsRNA molecule, is preferably capable of providing at least a partial reduction, more preferably a substantial reduction, or most preferably effective elimination of another agent such as a protein or mRNA, wherein the agent leaves the level of a second agent essentially unaffected, substantially unaffected, or partially unaffected.

As used herein, the terms “suppress,” “repress,” and “downregulate” when referring to the expression or activity of a nucleic acid molecule in a plant cell are used equivalently herein and mean that the level of expression or activity of the nucleic acid molecule in a plant, a plant part, or plant cell after applying a method of the present disclosure is lower than its expression or activity in the plant, part of the plant, or plant cell before applying the method, or compared to a control plant lacking a recombinant nucleic acid molecule of the disclosure.

The terms “suppressed,” “repressed” and “downregulated” as used herein are synonymous and mean herein lower, preferably significantly lower, expression or activity of the nucleic acid molecule to be expressed.

As used herein, a “suppression,” “repression,” or “downregulation” of the level or activity of an agent such as a protein, mRNA, or RNA means that the level or activity is reduced relative to a substantially identical plant, part of a plant, or plant cell grown under substantially identical conditions, lacking a recombinant nucleic acid molecule of the disclosure, for example, lacking the region complementary to at least a part of the precursor molecule of the miRNA, the recombinant construct or recombinant vector of the disclosure. As used herein, “suppression,” “repression,” or “downregulation” of the level or activity of an agent, such as, for example, a preRNA, mRNA, rRNA, tRNA, snoRNA, snRNA expressed by the target gene, and/or of the protein product encoded by it, means that the amount is reduced by 10% or more, for example, 20% or more, preferably 30% or more, more preferably 50% or more, even more preferably 70% or more, most preferably 80% or more, for example, 90%, relative to a cell or organism lacking a recombinant nucleic acid molecule of the disclosure.

Abiotic stress is a collective term for numerous extreme environmental parameters such as drought, high or low salinity, high or low temperature/light, and nutrient imbalances. The major agricultural crops (corn, rice, wheat, canola, and soybean) account for over half of total human caloric intake, giving their overall yield and quality importance. Abiotic stress causes more than 50% yield loss of the above-mentioned major crops (Wang et al., 2007, Planta, 218:1-14). Among the various abiotic stresses, drought is the major factor that limits crop productivity worldwide. Furthermore, drought is associated with increased susceptibility to various diseases. Abiotic-stress-induced dehydration or osmotic stress, in the form of reduced availability of water and disruption of turgor pressure, causes irreversible cellular damage. A water-limiting environment at various plant developmental stages may activate various physiological changes.

In soybean, drought can reduce yield by approximately 40%, with a critical period for water deprivation being the flowering stage and the period following flowering (Meckel et al., 1984, Agron. J., 75:1027-1031). Water deficit, salinity, and low/high temperatures are stresses that cause plant cellular dehydration, due to a transpiration rate that exceeds water uptake. Water use efficiency (WUE), defined as the amount of biomass accumulated per unit of water used, plays an important role in determining a plant's ability to tolerate drought stress. The higher the WUE of a plant, the higher the crop productivity and total biomass yield under drought conditions. Thus, efforts are made worldwide to increase the WUE of the most important crops and to reach the best yield performance under extreme water deficiency conditions. In an aspect, a transgenic plant of the present disclosure can show enhanced WUE relative to a control or wild type plant.

Drought is known to elicit a response in the plant that mainly affects root architecture (Jiang and Huang, 2001, Crop Sci., 41:1168-1173; Lopez-Bucio et al., 2003, Curr. Opin. Plant Biol., 6:280-287; Morgan and Condon, 1986, Aust. J. Plant Physiol., 13:523-532), causing activation of plant metabolic pathways driven to maximize water assimilation. Improvement of root architecture, e.g., making branched and longer roots, allows the plant to reach water and nutrient/fertilizer deposits located deeper in the soil by an increase in soil coverage. In soybean, there are correlations between drought resistance and various root traits such as dry weight, total length, volume, and number of lateral roots (Liu et al., 2005, Environ. Exp. Bot., 54:33-40). Thus, genes governing enhancement of root architecture may be used to improve drought tolerance. Furthermore, nitrogen (N₂) fixation in soybean is sensitive to drought conditions, resulting in reduced supply of N₂ for protein production, which is the critical seed product of the plant, and thus translates into lower crop yields (Purcell and King, 1996, J. Plant Nutr., 19:969-993). In an aspect, a transgenic plant of the present disclosure can have improved root architecture and improved drought tolerance relative to a control or wild-type plant. In another aspect, a transgenic plant of the present disclosure may have improved N₂ fixation relative to a control or wild-type plant.

High salt levels, or salinity, of the soil acts similarly to drought; it prevents roots from extracting water and nutrients and thus reduces the availability of arable land and crop production worldwide, since none of the top five food crops can tolerate excessive salt. Salinity causes a water deficit which leads to osmotic stress (similar to freezing and drought stress) and critically damages biochemical processes. Large land areas throughout the world naturally have high salt levels and thus are currently uncultivable. In regions that rely heavily on agricultural production, soil salinity is a significant problem expected to worsen due to growing population and extreme climatic changes. Since salt accumulates in the upper soil layer where seeds are placed, and may interfere with their germination, salt tolerance is of particular importance early in a plant's lifecycle. In an aspect, a transgenic plant of the present disclosure can have improved salt tolerance relative to a control or wild-type plant.

Temperature is a factor in germination of many crops. Seedlings as well as mature plants that are exposed to excess heat may experience heat shock, which may arise in various organs when transpiration is insufficient to overcome heat stress. Heat shock damages cellular structures and impairs membrane function and overall protein synthesis (except that of heat shock proteins). Heat stress often accompanies conditions of low water availability, such as drought, and the combined stress can fatally alter plant metabolism. Dehydration invokes survival strategies in plants that include structural (lower surface area) as well as cellular content (increase in oil and soluble material) modifications to prevent evaporation and water loss caused by heat, drought, or salinity. In an aspect, a transgenic plant of the present disclosure can have improved resistance to heat shock damage and improved germination relative to a control or wild-type plant.

Yield is affected by various factors, such as the number and size of the plant organs, plant architecture, grain's set length, number of filled grains, vigor (e.g., seedling), growth rate, root development, utilization of water, nutrients (e.g., nitrogen) and fertilizers, and stress tolerance. Seeds are also a source of sugars, oils, and metabolites used in industrial processes. The ability to increase plant yield, whether through increased dry matter accumulation rate, modified cellulose or lignin composition, increased stalk strength, enlarged meristem size, changed plant branching pattern, erectness of levees, increased fertilization efficiency, enhanced seed dry matter accumulation rate, modified seed development, enhanced seed filling, or increased content of oil, starch, or protein in the seeds would have many applications in agricultural and non-agricultural uses such as in the biotechnological production of pharmaceuticals, antibodies or vaccines. In an aspect, a transgenic plant of the present disclosure can have improved yield relative to a control or wild-type plant.

While not limited by a particular theory, two prevalent types of small RNAs, microRNAs (miRNAs) and small interfering RNAs (siRNAs) are similar in certain aspects and distinct in other aspects. For example, both promote specific down-regulation/silencing of a target gene through RNA interference (RNAi). Both miRNAs and siRNAs are oligonucleotides (20-24 bps) processed from longer RNA precursors by Dicer-like ribonucleases, although the source of their precursors is different (e.g., local single RNA molecules with imperfect stem-loop structures for miRNA, and long, double-stranded precursors potentially from bimolecular duplexes for siRNA). Additional characteristics that differentiate miRNAs from siRNAs are their sequence conservation level between related organisms (high in miRNAs, low to non-existent in siRNAs), regulation of genes unrelated to their locus of origin (typical for miRNAs, infrequent in siRNAs), and the genetic requirements for their respective functions are somewhat dissimilar in many organisms (Jones-Rhoades et al., 2006, Ann. Rev. Plant Biol., 57:19-53). While not limited by a particular theory, despite all their differences, miRNAs and siRNAs are overall chemically and functionally similar, and both are incorporated into silencing complexes, wherein they can guide post-transcriptional repression of multiple target genes, and thus function catalytically.

Various approaches are contemplated herein to regulate, either upregulate or downregulate, the expression or activity of a small RNA, including without limitation a miRNA, associated with abiotic stress. Upregulation of small RNA activity, including without limitation miRNA activity, can be achieved either permanently or transiently. Nucleic acid agents that down-regulate small RNA activity include, but are not limited to, target mimics, small RNA, including without limitation miRNA, resistant target genes, and a small RNA, including without limitation an mRNA, inhibitor.

This application provides and discloses small RNAs, including without limitation miRNAs, and their target genes that are involved in response and resistance to abiotic stresses, and methods of modulating expression or activity of these small RNAs, including without limitation miRNAs, and target genes. This application further provides transgenic plants, plant parts, e.g., seeds that have altered expression of these small RNAs, including without limitation miRNAs, and target genes and have improved abiotic stress tolerance. This application also provides methods of producing and growing transgenic plants or seeds that have improved abiotic stress tolerance. In specific embodiments, this application discloses small RNAs, including without limitation miRNAs, small RNA target genes, including without limitation miRNA target genes, small RNA, including without limitation miRNA, target mimics, engineered small RNA, including without limitation miRNA, resistant target genes, and uses thereof to improve plant drought tolerance.

In an aspect, the instant application discloses a method of improving abiotic stress tolerance in a soybean plant comprising transgenically expressing in the soybean plant a recombinant DNA construct comprising encoding a small RNA including, without limitation, miRNA, ta-siRNA, siRNA, activating RNA, nat-siRNA, hc-siRNA, cis-acting siRNA, lmiRNA, lsiRNA, easiRNA, or their respective intermediates and precursors.

In one aspect, the instant application discloses a method of improving abiotic stress tolerance in a soybean plant comprising transgenically expressing in the soybean plant a recombinant DNA construct comprising a heterologous promoter operably linked to a DNA encoding at least one miRNA precursor that yields a mature miRNA selected from the group consisting of a mature miR164 and a mature miR168. In certain aspects, a recombinant DNA construct may further comprise a transcription terminator. In certain aspects, a DNA encoding at least one miRNA precursor comprises a nucleotide sequence having at least 80%, 85%, 88%, 90%, 92%, 95%, 96%, 97%, 98%, or 99% sequence identity to a sequence selected from the group consisting of SEQ ID NOs: 1, 2, and 11-94. In another aspect, a DNA encoding at least one miRNA precursor comprises a nucleotide sequence selected from the group consisting of SEQ ID NOs: 1, 2, and 11-94.

In one aspect, the instant application discloses a method of improving abiotic stress tolerance in a soybean plant comprising transgenically expressing in the soybean plant a recombinant DNA construct comprising a heterologous promoter operably linked to a DNA encoding a miR397 target mimic, a miR408 target mimic, or a miR1093 target mimic. In certain aspects, a recombinant DNA construct may further comprise a transcription terminator. In certain aspects, a DNA encoding a miR397 target mimic, a miR408 target mimic, or a miR1093 target mimic comprises a nucleotide sequence having at least 80%, 85%, 88%, 90%, 92%, 95%, 96%, 97%, 98%, or 99% sequence identity to a sequence as set forth in SEQ ID NOs: 414, 415, or 416. In another aspect, a DNA encoding a miR397 target mimic, a miR408 target mimic, or a miR1093 target mimic comprises a nucleotide sequence as set forth in SEQ ID NOs: 414, 415, or 416. In another aspect, a DNA encoding a miR397 target mimic, a miR408 target mimic, or a miR1093 target mimic is set forth in SEQ ID NOs: 417, 418, or 419.

In a further aspect, the instant application discloses a method of improving abiotic stress tolerance in a soybean plant comprising transgenically expressing in the soybean plant a recombinant DNA construct comprising a heterologous promoter operably linked to a DNA encoding a miR397-, miR408-, or miR1093-resistant target gene, wherein the miR397-, miR408-, or miR1093-resistant target gene comprises an introduced silent mutation in a nucleotide sequence that is otherwise substantially identical to the nucleotide sequence of an endogenous gene that is natively regulated by miR397, miR408, or miR1093, and wherein the silent mutation prevents binding by a mature miR397, miR408, or miR1093 to a transcript of the miR397-, miR408-, or miR1093-resistant target gene. In certain aspects, a recombinant DNA construct may further comprise a transcription terminator. In certain aspects, a DNA encoding a miR397-, miR408-, or miR1093-resistant target gene comprises a nucleotide sequence having at least 80%, 85%, 88%, 90%, 92%, 95%, 96%, 97%, 98%, or 99% sequence identity to a sequence selected from the group consisting of SEQ ID NOs: 386-410. In another aspect, a DNA encoding a miR397-, miR408-, or miR1093-resistant target gene comprises a nucleotide sequence selected from the group consisting of SEQ ID NOs: 386-410.

In one aspect, a heterologous promoter used herein is selected from the group consisting of a constitutive promoter, a tissue-specific promoter, and an inducible promoter. In one aspect, a constitutive promoter is the CaMV 35S promoter. In another aspect, a promoter is an abiotic stress inducible promoter.

In one aspect, a method of improving abiotic stress tolerance in a soybean plant disclosed herein further involves transgenically expressing a recombinant DNA construct encoding a protein that provides tolerance to an herbicide selected from the group consisting of glyphosate, 2,4-dichloropropionic acid, bromoxynil, sulfonylurea, imidazolinone, triazolopyrimidine, pyrimidyloxybenzoates, phthalide, bialaphos, phosphinothricin, glufosinate, atrazine, dicamba, cyclohexanedione (sethoxydim), and aryloxyphenoxypropionate (haloxyfop). A recombinant DNA construct providing herbicide resistance and a recombinant DNA construct providing abiotic stress tolerance disclosed herein can be part of a single transgene which has a single site in the genome, or belong to separate transgenes that are located at different sites in the genome.

In an aspect, the instant application discloses a method of providing a plant with increased root branching or root depth comprising transgenically expressing in the soybean plant a recombinant DNA construct comprising encoding a small RNA including, without limitation, miRNA, ta-siRNA, siRNA, activating RNA, nat-siRNA, hc-siRNA, cis-acting siRNA, lmiRNA, lsiRNA, easiRNA or their respective intermediates and precursors.

In one aspect, the instant application discloses a method of providing a plant with increased root branching or root depth comprising transgenically expressing in the soybean plant a recombinant DNA construct comprising a heterologous promoter operably linked to a DNA encoding at least one miRNA precursor that yields a mature miRNA selected from the group consisting of a mature miR164 and a mature miR168. In certain aspects, a recombinant DNA construct may further comprise a transcription terminator. In some aspects, a DNA encoding at least one miRNA precursor comprises a nucleotide sequence having at least 80%, 85%, 88%, 90%, 92%, 95%, 96%, 97%, 98%, or 99% sequence identity to a sequence selected from the group consisting of SEQ ID NOs: 1, 2, and 11-94. In another aspect, a DNA encoding at least one miRNA precursor comprises a nucleotide sequence selected from the group consisting of SEQ ID NOs: 1, 2, and 11-94.

In one aspect, the instant application discloses a method of providing a plant with increased root branching or root depth comprising transgenically expressing in the soybean plant a recombinant DNA construct comprising a heterologous promoter operably linked to a DNA encoding a miR397 target mimic, a miR408 target mimic, or a miR1093 target mimic. In certain aspects, a recombinant DNA construct may further comprise a transcription terminator. In certain aspects, a DNA encoding a miR397 target mimic, a miR408 target mimic, or a miR1093 target mimic comprises a nucleotide sequence having at least 80%, 85%, 88%, 90%, 92%, 95%, 96%, 97%, 98%, or 99% sequence identity to a sequence as set forth in SEQ ID NOs: 414, 415, or 416. In another aspect, a DNA encoding a miR397 target mimic, a miR408 target mimic, or a miR1093 target mimic comprises a nucleotide sequence as set forth in SEQ ID NOs: 414, 415, or 416. In another aspect, a DNA encoding a miR397 target mimic, a miR408 target mimic, or a miR1093 target mimic is set forth in SEQ ID NOs: 417, 418, or 419.

In a further aspect, the instant application discloses a method of providing a plant with increased root branching or root depth comprising transgenically expressing in the soybean plant a recombinant DNA construct comprising a heterologous promoter operably linked to a DNA encoding a miR397-, miR408-, or miR1093-resistant target gene, wherein the miR397-, miR408-, or miR1093-resistant target gene comprises an introduced silent mutation in a nucleotide sequence that is otherwise substantially identical to the nucleotide sequence of an endogenous gene that is natively regulated by miR397, miR408, or miR1093, and wherein the silent mutation inhibits binding by a mature miR397, miR408, or miR1093 to a transcript of the miR397-, miR408-, or miR1093-resistant target gene. In certain aspects, a recombinant DNA construct may further comprise a transcription terminator. In certain aspects, a DNA encoding a miR397-, miR408-, or miR1093-resistant target gene comprises a nucleotide sequence having at least 80%, 85%, 88%, 90%, 92%, 95%, 96%, 97%, 98%, or 99% sequence identity to a sequence selected from the group consisting of SEQ ID NOs: 386-410. In another aspect, a DNA encoding a miR397-, miR408-, or miR1093-resistant target gene comprises a nucleotide sequence selected from the group consisting of SEQ ID NOs: 386-410.

In one aspect, a heterologous promoter providing a plant with increased root branching or root depth is selected from the group consisting of a constitutive promoter, a tissue-specific promoter, and an inducible promoter. In one aspect, a constitutive promoter is the CaMV 35S promoter. In another aspect, a promoter is an abiotic stress inducible promoter.

In one aspect, a method of providing a plant with increased root branching or root depth disclosed herein further involves transgenically expressing a recombinant DNA construct encoding a protein that provides tolerance to an herbicide selected from the group consisting of glyphosate, 2,4-dichloropropionic acid, bromoxynil, sulfonylurea, imidazolinone, triazolopyrimidine, pyrimidyloxybenzoates, phthalide, bialaphos, phosphinothricin, glufosinate, atrazine, dicamba, cyclohexanedione (sethoxydim), and aryloxyphenoxypropionate (haloxyfop). A recombinant DNA construct providing herbicide resistance and a recombinant DNA construct providing abiotic stress tolerance disclosed herein can be part of a single transgene which has a single site in the genome, or belong to separate transgenes that are located at different sites in the genome.

In another aspect, the instant application discloses a method of producing a transgenic soybean plant, the method comprising transforming a soybean plant cell with a transgene comprising a heterologous promoter operably linked to at least one DNA encoding a mature miRNA, comprising a sequence having at least 80%, 85%, 88%, 90%, 92%, 95%, 96%, 97%, 98%, or 99% sequence identity to a sequence selected from the group consisting of SEQ ID NOs: 1, 2, and 11-94, and producing a transgenic soybean plant from the transformed cell, wherein the transgenic soybean plant has improved drought tolerance compared to a control soybean plant lacking the transgene. In certain aspects, the transgene may further comprise a transcription terminator sequence.

In another aspect, the instant application discloses a method of producing a transgenic soybean plant, the method comprising transforming a soybean plant cell with a transgene comprising a heterologous promoter operably linked to at least one DNA encoding a mature miRNA, comprising a sequence having at least 80%, 85%, 88%, 90%, 92%, 95%, 96%, 97%, 98%, or 99% sequence identity to a sequence selected from the group consisting of SEQ ID NOs: 1, 2, and 11-94, and producing a transgenic soybean plant from the transformed cell, wherein the transgenic soybean plant has increased root branching or root depth compared to a control soybean plant lacking the transgene. In certain aspects, the transgene may further comprise a transcription terminator sequence.

In another aspect, the instant application discloses a method of producing a transgenic soybean plant, the method comprising transforming a soybean plant cell with a transgene comprising a heterologous promoter operably linked to at least one DNA encoding a pre-miRNA or target mimic RNA, comprising a sequence having at least 80%, 85%, 88%, 90%, 92%, 95%, 96%, 97%, 98%, or 99% sequence identity to a sequence selected from the group consisting of SEQ ID NOs: 1, 2 and 11-94, and producing a transgenic soybean plant from the transformed cell, wherein the transgenic soybean plant has improved drought tolerance compared to a control soybean plant lacking the transgene. In certain aspects, the transgene may further comprise a transcription terminator sequence.

In another aspect, the instant application discloses a method of producing a transgenic soybean plant, the method comprising transforming a soybean plant cell with a transgene comprising a heterologous promoter operably linked to at least one DNA encoding a pre-miRNA or target mimic RNA, comprising a sequence having at least 80%, 85%, 88%, 90%, 92%, 95%, 96%, 97%, 98%, or 99% sequence identity to a sequence selected from the group consisting of SEQ ID NOs: 1, 2, and 11-94, and producing a transgenic soybean plant from the transformed cell, wherein the transgenic soybean plant has increased root branching or root depth compared to a control soybean plant lacking the transgene. In certain aspects, the transgene may further comprise a transcription terminator sequence.

In another aspect, the instant application discloses a method of producing a transgenic soybean plant, the method comprising transforming a soybean plant cell with a transgene comprising a heterologous promoter operably linked to at least one DNA having at least 80%, 85%, 88%, 90%, 92%, 95%, 96%, 97%, 98%, or 99% sequence identity to a sequence selected from the group consisting of SEQ ID NOs: 417-419 and 386-410, and producing a transgenic soybean plant from the transformed cell, wherein the transgenic soybean plant has improved drought tolerance compared to a control soybean plant lacking the transgene. In certain aspects, the transgene may further comprise a transcription terminator sequence.

In another aspect, the instant application discloses a method of producing a transgenic soybean plant, the method comprising transforming a soybean plant cell with a transgene comprising a heterologous promoter operably linked to at least one DNA having at least 80%, 85%, 88%, 90%, 92%, 95%, 96%, 97%, 98%, or 99% sequence identity to a sequence selected from the group consisting of SEQ ID NOs: 417-419 and 386-410, and producing a transgenic soybean plant from the transformed cell, wherein the transgenic soybean plant has increased root branching or root depth compared to a control soybean plant lacking the transgene. In certain aspects, the transgenic soybean plant may further comprise a transcription terminator sequence.

In one aspect, the instant application discloses a transgenic soybean plant, or part thereof, comprising a transgene that encodes a mature miRNA comprising a sequence having at least 80%, 85%, 88%, 90%, 92%, 95%, 96%, 97%, 98%, or 99% sequence identity to a sequence selected from the group consisting of SEQ ID NOs: 1, 2, and 11-94, wherein the transgenic soybean plant has improved drought tolerance compared to a non-transgenic control soybean plant.

In one aspect, the instant application discloses a transgenic soybean plant, or part thereof, comprising a transgene that encodes a mature miRNA comprising a sequence having at least 80%, 85%, 88%, 90%, 92%, 95%, 96%, 97%, 98%, or 99% sequence identity to a sequence selected from the group consisting of SEQ ID NOs: 1, 2, and 11-94, wherein the transgenic soybean plant has increased root branching or root depth compared to a non-transgenic control soybean plant.

In another aspect, the instant application discloses a transgenic soybean plant, or part thereof, comprising a transgene that encodes a miR397 target mimic, a miR408 target mimic, or a miR1093 target mimic, and the transgenic soybean plant has improved drought tolerance compared to a non-transgenic control soybean plant.

In another aspect, the instant application discloses a transgenic soybean plant, or part thereof, comprising a transgene that encodes a miR397 target mimic, a miR408 target mimic, or a miR1093 target mimic, and the transgenic soybean plant has increased root branching or root depth compared to a non-transgenic control soybean plant.

In a further aspect, the instant application discloses a transgenic soybean plant, or part thereof, comprising a transgene that encodes a miR397-, miR408-, or miR1093-resistant target gene, wherein the miR397-, miR408-, or miR1093-resistant target gene comprises a sequence having at least 80%, 85%, 88%, 90%, 92%, 95%, 96%, 97%, 98%, or 99% sequence identity to a sequence selected from the group consisting of SEQ ID NOs: 386-410, and the transgenic soybean plant has improved drought tolerance compared to a non-transgenic control soybean plant.

In a further aspect, the instant application discloses a transgenic soybean plant, or part thereof, comprising a transgene that encodes a miR397-, miR408-, or miR1093-resistant target gene, wherein the miR397-, miR408-, or miR1093-resistant target gene comprises a sequence having at least 80%, 85%, 88%, 90%, 92%, 95%, 96%, 97%, 98%, or 99% sequence identity to a sequence selected from the group consisting of SEQ ID NOs: 386-410, and the transgenic soybean plant has increased root branching or root depth compared to a non-transgenic control soybean plant.

In one aspect, the instant application discloses a method of producing a transgenic plant, the method comprising transforming a plant cell with a transgene comprising a sequence having at least 80%, 85%, 88%, 90%, 92%, 95%, 96%, 97%, 98%, or 99% sequence identity to a sequence selected from the group consisting of SEQ ID NOs: 1 to 262, 317 to 370, and 380 to 419, and producing a transgenic plant from the transformed cell, wherein the transgenic plant has improved drought tolerance compared to a control plant lacking the transgene.

In one aspect, the instant application discloses a method of producing a transgenic plant, the method comprising transforming a plant cell with a transgene encoding a polypeptide sequence having at least 80%, 85%, 88%, 90%, 92%, 95%, 96%, 97%, 98%, or 99% sequence identity to a sequence selected from the group consisting of SEQ ID NOs: 263 to 317 and 371 to 379, and producing a transgenic plant from the transformed cell, wherein the transgenic plant has improved drought tolerance compared to a control plant lacking the transgene.

In one aspect, the instant application discloses a method of producing a transgenic plant, the method comprising transforming a plant cell with a transgene encoding a small RNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, and producing a transgenic plant from the transformed cell, wherein the transgenic plant has improved drought tolerance compared to a control plant lacking the transgene. In a further aspect, a method of the instant application further comprises collecting a seed from the transgenic plant.

In another aspect, a method of producing a transgenic plant disclosed herein produces a transgenic plant having a transgene stably integrated into the nuclear genome of the transgenic plant. In a further aspect, a method of producing a transgenic plant disclosed herein produces a transgenic plant having a transgene stably integrated into the chloroplast of the transgenic plant.

In a further aspect, a method of producing a transgenic plant disclosed herein produces a transgenic plant having improved drought tolerance measured by an increase of at least 1% in water use efficiency (WUE) when the transgenic and control plants grow under similar drought conditions, and the WUE is measured by the amount of biomass accumulated per unit of water used.

In another aspect, a method of producing a transgenic plant disclosed herein uses a transgene comprising a promoter selected from the group consisting of a constitutive promoter, a tissue-specific promoter, and an inducible promoter. In one aspect, a constitutive promoter is the CaMV 35S promoter. In another aspect, a promoter is an abiotic stress inducible promoter.

In one aspect, the instant application discloses a method of producing a transgenic plant, the method comprising transforming a plant cell with a transgene encoding a target nucleic acid molecule that is complementary to a small RNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, and producing a transgenic plant from the transformed cell, wherein the transgenic plant has improved drought tolerance compared to a non-transgenic control plant. In another aspect, a transgene used in a method of producing a transgenic plant disclosed herein expresses a small RNA target nucleic acid or in an articular aspect a miRNA molecule that is substantially resistant to small RNA-mediated cleavage. In a further aspect, a small RNA target nucleic acid molecule used in a method disclosed herein is constitutively expressed.

In one aspect, a small RNA, or in a particular aspect, a miRNA target nucleic acid molecule used in a method disclosed herein comprises a sequence having at least 80%, 85%, 88%, 90%, 92%, 95%, 96%, 97%, 98%, or 99% sequence identity to a sequence selected from the group consisting of SEQ ID NOs: 317 to 370.

In another aspect, a small RNA, or in a particular aspect, a miRNA target nucleic acid molecule used in a method disclosed herein encodes a polypeptide having at least 80%, 85%, 88%, 90%, 92%, 95%, 96%, 97%, 98%, or 99% sequence identity to a sequence selected from the group consisting of SEQ ID NOs: 263 to 316.

In one aspect, a small RNA target, or in a particular aspect, a miRNA nucleic acid molecule used in a method disclosed herein is a target mimic. In one aspect, a target mimic is capable of binding the small RNA, or in a particular aspect, a miRNA without being cleaved, and thus sequestering the small RNA, or in a particular aspect, a miRNA and preventing the small RNA, or in a particular aspect, a miRNA from binding other target molecules of the small RNA, or in a particular aspect, a miRNA. In another aspect, a target mimic comprises extra nucleotides within a small RNA binding, or in a particular aspect, a miRNA site between two nucleotides that are complementary to bases 10 and 11 of the small RNA, or in a particular aspect, a miRNA. In a further aspect, extra nucleotides contained in a target mimic consist of Adenine, Uracil, and Cytosine (AUC).

In one aspect, a target mimic of a small RNA, miRNA or a small or miRNA-resistant target nucleic acid molecule used herein is operably linked to a promoter naturally associated with a precursor of the small RNA or in a particular aspect miRNA. In this way, without being bound to any scientific theory or mechanism, the target mimic or small RNA-, or in a particular aspect, miRNA-resistant target nucleic acid molecule will be expressed under the same circumstances as the small RNA, or in a particular aspect, miRNA. In turn, the target mimic or small RNA-resistant target nucleic acid molecule will compete with an endogenous target RNA for binding to the small RNA, or in a particular aspect, miRNA, and thus prevent cleavage or downregulation of the endogenous target RNA.

In one aspect, the instant application discloses a method of producing a transgenic plant, the method comprising transforming a plant cell with a transgene that regulates the expression of a target nucleic acid molecule that is complementary to a small RNA, or in a particular aspect, a miRNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, and producing a transgenic plant from the transformed cell, wherein the transgenic plant has improved drought tolerance compared to a non-transgenic control plant. In another aspect, a transgene used in a method disclosed herein regulates the expression of a small RNA, or in a particular aspect, a miRNA target nucleic acid molecule via an artificial miRNA complementary with the small RNA target nucleic acid molecule. In a further aspect, a transgene used in a method disclosed herein regulates the expression of a small RNA, or in a particular aspect, a miRNA target nucleic acid molecule via RNA interference.

In one aspect, the instant application discloses a method of improving plant drought tolerance, the method comprising transforming a plant with an exogenous nucleic acid molecule comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136. In another aspect, the instant application further discloses a plant having improved drought tolerance, and comprising an exogenous nucleic acid molecule comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136.

In one aspect, an exogenous nucleic acid molecule used herein is or encodes a small RNA, or in a particular aspect a miRNA, which modulates abiotic stress tolerance of a plant. In a further aspect, an exogenous nucleic acid molecule used herein is or encodes a dsRNA molecule. In another aspect, an exogenous nucleic acid molecule used herein is or encodes an artificial miRNA. In a further aspect, an exogenous nucleic acid molecule used herein is or encodes an siRNA. In one aspect, an exogenous nucleic acid molecule used herein is or encodes a precursor of a small RNA. In another aspect, an exogenous nucleic acid molecule used herein is or encodes a precursor of a miRNA or siRNA. In one aspect, an exogenous nucleic acid molecule used herein is a naturally-occurring molecule. In another aspect, an exogenous nucleic acid molecule used herein is a synthetic molecule.

In one aspect, an exogenous nucleic acid molecule used herein is or encodes a stem-loop precursor of a small RNA or in a particular aspect a miRNA, comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136. A stem-loop precursor used herein comprises a sequence having at least 60%, 65%, 70%, 75%, 80%, 85%, 88%, 90%, 92%, 95%, 96%, 97%, 98%, or 99% sequence identity to a sequence selected from the group consisting of SEQ ID NOs: 6 to 10 and 137 to 262.

In one aspect, an exogenous nucleic acid molecule used herein is naked RNA or expressed from a nucleic acid expression construct, where it is operably linked to a regulatory sequence.

In one aspect, a recombinant DNA construct or a transgene disclosed herein further comprises a transcription terminator.

In one aspect, agrobacterium-mediated transformation is used in a method disclosed. In another aspect, a transgenic plant disclosed herein is produced by agrobacterium-mediated transformation.

In one aspect, a transgenic plant, or part thereof, disclosed herein is homozygous for the transgene. In another aspect, a transgenic plant, or part thereof, disclosed herein is heterozygous for the transgene.

In one aspect, a transgenic plant, or part thereof, disclosed herein has a single insertion of the transgene. In one aspect, a transgenic plant, or part thereof, disclosed herein has multiple insertions of the transgene at different genomic loci or at a single site in a tandem manner.

In one aspect, a transgenic plant disclosed herein comprises one or more additional enhanced traits. In one aspect, a transgenic plant disclosed herein comprises increased vigor over that of a control plant. In another aspect, a transgenic plant disclosed herein comprises higher yield than a control plant.

In one aspect, the transgenic expression of miR164 or miR168 causes a reduction in the expression or activity of at least one target gene of miR164 or miR168 in at least one cell type. In another aspect, the transgenic expression of miR164 or miR168 causes a partial reduction in the expression or activity of at least one target gene of miR164 or miR168 in at least one cell type. In a further aspect, the transgenic expression of miR164 or miR168 causes a substantial reduction in the expression or activity of at least one target gene of miR164 or miR168 in at least one cell type. In another aspect, the transgenic expression of miR164 or miR168 causes an effective elimination of the expression or activity of at least one target gene of miR164 or miR168 in at least one cell type.

In one aspect, the transgenic expression of miR164 or miR168 causes a reduction in one or more cell types of the expression or activity of one gene encoding an amino acid sequence having at least 80%, 85%, 88%, 90%, 92%, 95%, 96%, 97%, 98%, or 99% sequence identity to a sequence selected from the group consisting of SEQ ID NOs: 263 to 317 and 371 to 379.

In one aspect, the transgenic expression of miR164 or miR168 causes a substantial reduction in one or more cell types of the expression or activity of one gene encoding an amino acid sequence having at least 80%, 85%, 88%, 90%, 92%, 95%, 96%, 97%, 98%, or 99% sequence identity to a sequence selected from the group consisting of SEQ ID NOs: 263 to 317 and 371 to 379.

In one aspect, the transgenic expression of miR164 or miR168 causes in one or more cell type an effective elimination of the expression or activity of one gene encoding an amino acid sequence having at least 80%, 85%, 88%, 90%, 92%, 95%, 96%, 97%, 98%, or 99% sequence identity to a sequence selected from the group consisting of SEQ ID NOs: 263 to 317 and 371 to 379.

In one aspect, the transgenic expression of a miR397 mimic, a miR408 mimic, or a miR1093 mimic causes an increase in the expression or activity of at least one target gene of miR397, miR408, or miR1093 in at least one cell type.

In another aspect, the transgenic expression of a miR397 mimic, a miR408 mimic, or a miR1093 mimic causes an increase of at least 20%, 40%, 60%, 80%, 100%, 200%, 300%, 400%, or 500% in the expression or activity of at least one gene encoding an amino acid sequence having at least 80%, 85%, 88%, 90%, 92%, 95%, 96%, 97%, 98%, or 99% sequence identity to a sequence selected from the group consisting of SEQ ID NOs: 263 to 317 and 371 to 379.

In a further aspect, an exogenous nucleic acid molecule used herein is a synthetic single-stranded nucleic acid molecule known as a miRNA inhibitor. A miRNA inhibitor is typically between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, a miRNA inhibitor has a sequence (from 5′ to 3′) that is or is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% complementary, or any range derivable therein, to the 5′ to 3′ sequence of a mature miRNA, particularly a mature, naturally-occurring miRNA.

The instant application further discloses a transgenic plant or part thereof produced by a method disclosed herein. In an aspect, a transgenic plant or part thereof disclosed herein is a hybrid plant.

In one aspect, a transgenic plant, or part thereof, disclosed herein comprises a transgene comprising a sequence having at least 80%, 85%, 88%, 90%, 92%, 95%, 96%, 97%, 98%, or 99% sequence identity to a sequence selected from the group consisting of SEQ ID NOs: 1 to 262, 317 to 370, and 380 to 419, wherein the transgenic plant has improved drought tolerance compared to a control plant lacking the transgene.

In another aspect, a transgenic plant, or part thereof, disclosed herein comprises a transgene encoding a polypeptide sequence having at least 80%, 85%, 88%, 90%, 92%, 95%, 96%, 97%, 98%, or 99% sequence identity to a sequence selected from the group consisting of SEQ ID NOs: 263 to 317 and 371 to 379, wherein the transgenic plant has improved drought tolerance compared to a control plant lacking the transgene.

In one aspect, a transgenic plant, or part thereof, disclosed herein comprises a transgene encoding a small RNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, wherein the transgenic plant has improved drought tolerance compared to a control plant lacking the transgene.

In another aspect, a transgenic plant, or part thereof, disclosed herein comprises a transgene stably integrated into the nuclear genome of the transgenic plant. In a further aspect, a method of producing a transgenic plant disclosed herein produces a transgenic plant having a transgene stably integrated into the chloroplast of the transgenic plant.

In a further aspect, a transgenic plant, or part thereof, disclosed herein has improved drought tolerance measured by an increase of at least 1% in water use efficiency (WUE) when the transgenic and control plants grow under similar drought conditions, and the WUE is measured by the amount of biomass accumulated per unit of water used.

In another aspect, a transgenic plant, or part thereof, disclosed herein comprises a transgene comprising a promoter selected from the group consisting of a constitutive promoter, a tissue-specific promoter, and an inducible promoter. In one aspect, a constitutive promoter is the CaMV 35S promoter. In another aspect, a promoter is an abiotic stress-inducible promoter.

In one aspect, a transgenic plant, or part thereof, disclosed herein comprises a transgene encoding a small RNA target nucleic acid molecule that is complementary to a small RNA comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, wherein the transgenic plant has improved drought tolerance compared to a non-transgenic control plant.

In another aspect, a transgenic plant, or part thereof, disclosed herein comprises a small RNA target nucleic acid molecule, or in a particular aspect a miRNA, that is substantially resistant to small RNA-mediated cleavage. In a further aspect, a small RNA, or in a particular aspect a miRNA, target nucleic acid molecule used in a method disclosed herein is constitutively expressed. In one aspect, a small RNA target, or in a particular aspect a miRNA, nucleic acid molecule produced from a transgene of a transgenic plant, or part thereof, disclosed herein comprises a sequence having at least 80%, 85%, 88%, 90%, 92%, 95%, 96%, 97%, 98%, or 99% sequence identity to a sequence selected from the group consisting of SEQ ID NOs: 317 to 370.

In another aspect, a small RNA, or in a particular aspect a miRNA, target nucleic acid molecule produced by a transgene in a transgenic plant, or part thereof, disclosed herein encodes a polypeptide having at least 80%, 85%, 88%, 90%, 92%, 95%, 96%, 97%, 98%, or 99% sequence identity to a sequence selected from the group consisting of SEQ ID NOs: 263 to 316.

In one aspect, a transgenic plant, or part thereof, disclosed herein comprises a transgene encoding a small RNA, or in a particular aspect a miRNA, target mimic. In one aspect, a small RNA, or in a particular aspect a miRNA, target mimic is expressed in a transgenic plant, or part thereof, disclosed herein, and is capable of binding a small RNA, or in a particular aspect a miRNA, without being cleaved, and thus sequestering the small RNA, or in a particular aspect a miRNA, and preventing the small RNA, or in a particular aspect a miRNA, from binding other target molecules of the small RNA, or in a particular aspect a miRNA. In another aspect, a target mimic comprises extra nucleotides within a small RNA, or in a particular aspect a miRNA, binding site between two nucleotides that are complementary to bases 10 and 11 of the small RNA, or in a particular aspect a miRNA. In a further aspect, extra nucleotides contained in a target mimic consist of Adenine, Uracil, and Cytosine (AUC).

In one aspect, the instant application discloses a transgenic plant, or part thereof, comprising a transgene that regulates the expression of a small RNA, or in a particular aspect a miRNA, target nucleic acid molecule that is complementary to a small RNA, or in a particular aspect a miRNA, comprising a sequence having at least 80%, 85%, 88%, 90%, 92%, 95%, 96%, 97%, 98%, or 99% sequence identity to a sequence selected from the group consisting of SEQ ID NOs: 1 to 5 and 11 to 136, wherein the transgenic plant has improved drought tolerance compared to a non-transgenic control plant. In another aspect, a transgenic plant, or part thereof, disclosed herein comprises a transgene encoding an artificial miRNA complementary with a small RNA, or in a particular aspect a miRNA, target nucleic acid molecule. In a further aspect, a transgenic plant, or part thereof, disclosed herein comprises a transgene that regulates the expression of a small RNA target nucleic acid molecule via RNA interference.

In one aspect, a transgenic plant, or part thereof, disclosed herein further comprises a transgene encoding a protein that provides tolerance to an herbicide. A transgene providing herbicide resistance and a transgene provide an enhanced trait disclosed herein can be part of a single transgene which has a single site in the genome, or belong to separate transgenes that are located at different sites in the genome.

In another aspect, a transgenic plant, or part thereof, disclosed herein is resistant to an herbicide selected from the group consisting of glyphosate, 2,4-dichloropropionic acid, bromoxynil, sulfonylurea, imidazolinone, triazolopyrimidine, pyrimidyloxybenzoates, phthalide, bialaphos, phosphinothricin, glufosinate, atrazine, dicamba, cyclohexanedione (sethoxydim), and aryloxyphenoxypropionate (haloxyfop).

In a further aspect, a transgenic plant part disclosed herein is selected from the group consisting of a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast.

In one aspect, the instant disclosure provides a non-reproductive plant cell or part, for example, a leaf, a stem, a root, a pedicel, a cotyledon, or a hypocotyl. In another aspect, the instant disclosure provides a plant part or cell that cannot regenerate into a complete plant. In another aspect, the instant disclosure provides a plant part or cell that cannot regenerate into a new plant as a means to reproduce or propagate a plant.

In one aspect, the instant disclosure provides a population of transgenic plants provided herein which have improved drought tolerance compared to a non-transgenic control plant. In another aspect, the instant disclosure also provides a container of transgenic seeds provided herein which have improved drought tolerance compared to a non-transgenic control seed.

In one aspect, the instant disclosure provides a population of transgenic soybean plants provided herein which have improved drought tolerance compared to a non-transgenic control soybean plant. In another aspect, the instant disclosure also provides a container of transgenic soybean seeds provided herein which have improved drought tolerance compared to a non-transgenic control soybean seeds.

A container of transgenic soybean seeds of the instant disclosure may contain any number, weight, or volume of seeds. For example, a container can contain at least, or greater than, 100, 200, 300, 400, 500, 600, 700, 800, 900, 1000, 1500, 2000, 2500, 3000, 3500, 4000 or more seeds. Alternatively, the container can contain at least, or greater than, 1 ounce, 5 ounces, 10 ounces, 1 pound, 2 pounds, 3 pounds, 4 pounds, 5 pounds or more seeds. Containers of soybean seeds may be any container available in the art. By way of non-limiting example, a container may be a box, a bag, a packet, a pouch, a tape roll, or a tube.

In one aspect, the instant disclosure also provides a food or feed comprising the plants or a portion thereof of the present disclosure. In a further aspect, a transgenic plants, or part thereof disclosed herein is comprised in a food or feed product (e.g., dry, liquid, paste). A food or feed product is any ingestible preparation containing the transgenic plants, or parts thereof, of the present disclosure, or preparations made from these plants. Thus, the plants or preparations are suitable for human (or animal) consumption, e.g., the transgenic plants or parts thereof are more readily digested. Feed products of the present disclosure further include an oil or a beverage adapted for animal consumption.

In another aspect, a transgenic plant disclosed herein can be used directly as feed products, or alternatively can be incorporated or mixed with feed products for consumption. Furthermore, the food or feed products can be processed or used as is. Exemplary feed products comprising the transgenic plants, or parts thereof, include, but are not limited to, grains; cereals, such as oats, e.g., black oats, barley, wheat, or rye; sorghum; corn; vegetables; leguminous plants, especially soybeans, root vegetables, and cabbage; or green forage, such as grass or hay.

Also contemplated in the present disclosure are hybrids produced from a transgenic plant disclosed herein.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” promoter is one which is capable of directing a level of transcription of an operably linked nucleic acid sequence in the presence of a stimulus (e.g., heat shock, chemicals, light, etc.) which is different from the level of transcription of the operably linked nucleic acid sequence in the absence of the stimulus. In an aspect, a transgenic plant of the present disclosure can include DNA constructs having constitutive or regulatable promoters that provide transient or constitutive expression of one or more siRNAs, siRNA precursors, miRNAs or miRNA precusors. In certain aspects, transgenic plants can include DNA constructs having both a constitutive promoter and a regulatable promoter.

Any promoter that functions in a plant cell to cause the production of a RNA molecule, such as those promoters described herein, without limitation, can be used. In a preferred embodiment, the promoter is a plant promoter.

Tissue-specific or cell-type-specific expression of a nucleic acid molecule disclosed herein can be achieved by tissue-specific or cell-type-specific promoters. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue.

Root-specific promoters may also be used. An example of such a promoter is the promoter for the acid chitinase gene (Samac et al., Plant Mol. Biol., 25:587-596 (1994)). Expression in root tissue could also be accomplished by utilizing the root specific subdomains of the CaMV35S promoter that have been identified (Lam et al., PNAS USA, 86:7890-7894 (1989)). Other root-cell-specific promoters include those reported by Conkling et al. (Plant Physiol., 93:1203-1211 (1990)).

In an aspect according to the instant specification, tissue-specific or cell-type-specific expression of a nucleic acid molecule disclosed herein can be achieved by tissue-specific or cell-type-specific enhancer sequences. The term “tissue specific” as it applies to an enhancer refers to an enhancer that is capable of directing selective expression of a nucleotide sequence of interest to a specific type of tissue (e.g., petals) in the relative absence of expression of the same nucleotide sequence of interest in a different type of tissue (e.g., roots). The term “cell type specific” as applied to an enhancer refers to an enhancer which is capable of directing selective expression of a nucleotide sequence of interest in a specific type of cell in the relative absence of expression of the same nucleotide sequence of interest in a different type of cell within the same tissue. The term “cell type specific” when applied to an enhancer also means an enhancer capable of promoting selective expression of a nucleotide sequence of interest in a region within a single tissue.

In a further aspect, a nucleic acid molecule disclosed herein can be applied to the surface of a plant (e.g., leaf surface), or treated to a plant seed to induce a physiological response in a plant, including without limitation, providing improved tolerance to abiotic stresses (e.g., drought or salinity). Methods and composition components for applying a nucleic acid molecule to the surface of a plant was disclosed in US 2011/0296556 A1, which publication is incorporated by reference in its entirety.

Any commercially or scientifically valuable plant is envisaged in accordance with these embodiments of the disclosure. Plants that are particularly useful in the methods of the disclosure include all plants which belong to the super family Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp., Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lo tonus bainesli, Lotus spp., Macro tyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canadensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp., Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, eucalyptus, a tree, an ornamental plant, a perennial grass, and a forage crop. Alternatively algae and other non-Viridiplantae can be used for the methods of the present disclosure.

In aspects according to the present disclosure, a transgenic plant may be any plant. In certain aspects, a transgenic plant may preferably be a soybean plant.

Genetic material provided in the present disclosure may be introduced into any species, for example, without limitation, monocotyledons or dicotyledons, including, but not limited to alfalfa, apple, Arabidopsis, banana, barley, Brassica campestris, canola, castor bean, chrysanthemum, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, perennial, Phaseolus, potato, rapeseed, rice, rye, ryegrass, safflower, sesame, sorghum, soybean, sugarbeet, sugarcane, sunflower, tobacco, tomato, turfgrass, or wheat (Christou, I N O: Particle Bombardment for Genetic Engineering of Plants, Biotechnology Intelligence Unit. Academic Press, San Diego, Calif. (1996)), with alfalfa, Arabidopsis, Brassica campestris, canola, castor bean, corn, cotton, cottonseed, crambe, flax, linseed, mustard, oil palm, oilseed rape, peanut, potato, rapeseed, sunflower, sesame, soybean, sunflower, tobacco, tomato, and wheat preferred, and Brassica campestris, canola, corn, oil palm, oilseed rape, peanut, rapeseed, safflower, soybean, and sunflower more preferred. In a more preferred aspect, genetic material is transferred into canola. In another more preferred aspect, genetic material is transferred into oilseed rape. In another particularly preferred embodiment, genetic material is transferred into soybean or corn.

A transgenic soybean plant of the instant disclosure can be from any maturity group or any variety. Any enhanced plant trait of the present disclosure, for example, the improved abiotic stress tolerance may be introduced into an elite Glycine max line. An “elite line” is any line that has resulted from breeding and selection for superior agronomic performance. Examples of elite lines are lines that are commercially available to farmers or soybean breeders such as HARTZ™ variety H4994, HARTZ™ variety H5218, HARTZ™ variety H5350, HARTZ™ variety H5545, HARTZ™ variety H5050, HARTZ™ variety H5454, HARTZ™ variety H5233, HARTZ™ variety H5488, HARTZ™ variety HLA572, HARTZ™ variety H6200, HARTZ™ variety H6104, HARTZ™ variety H6255, HARTZ™ variety H6586, HARTZ™ variety H6191, HARTZ™ variety H7440, HARTZ™ variety H4452 ROUNDUP READY™, HARTZ™ variety H4994 ROUNDUP READY™, HARTZ™ variety H4988 ROUNDUP READY™, HARTZ™ variety H5000 ROUNDUP READY™ HARTZ™ variety H5147 ROUNDUP READY™, HARTZ™ variety H5247 ROUNDUP READY™, HARTZ™ variety H5350 ROUNDUP READY™, HARTZ™ variety H5545 ROUNDUP READY™, HARTZ™ variety H5855 ROUNDUP READY™, HARTZ™ variety H5088 ROUNDUP READY™, HARTZ™ variety H5164 ROUNDUP READY™, HARTZ™ variety H5361 ROUNDUP READY™ HARTZ™ variety H5566 ROUNDUP READY™, HARTZ™ variety H5181 ROUNDUP READY™, HARTZ™ variety H5889 ROUNDUP READY™, HARTZ™ variety H5999 ROUNDUP READY™, HARTZ™ variety H6013 ROUNDUP READY™, HARTZ™ variety H6255 ROUNDUP READY™, HARTZ™ variety H6454 ROUNDUP READY™, HARTZ™ variety H6686 ROUNDUP READY™ HARTZ™ variety H7152 ROUNDUP READY™, HARTZ™ variety H7550 ROUNDUP READY™, HARTZ™ variety H8001 ROUNDUP READY™ (HARTZ SEED, Stuttgart, Ark., USA); A0868, AG0202, AG0401, AG0803, AG0901, A1553, A1900, AG1502, AG1702, AG1901, A1923, A2069, AG2101, AG2201, AG2205, A2247, AG2301, A2304, A2396, AG2401, AG2501, A2506, A2553, AG2701, AG2702, AG2703, A2704, A2833, A2869, AG2901, AG2902, AG2905, AG3001, AG3002, AG3101, A3204, A3237, A3244, AG3301, AG3302, AG3006, AG3203, A3404, A3469, AG3502, AG3503, AG3505, AG3305, AG3602, AG3802, AG3905, AG3906, AG4102, AG4201, AG4403, AG4502, AG4603, AG4801, AG4902, AG4903, AG5301, AG5501, AG5605, AG5903, AG5905, A3559, AG3601, AG3701, AG3704, AG3750, A3834, AG3901, A3904, A4045 AG4301, A4341, AG4401, AG4404, AG4501, AG4503, AG4601, AG4602, A4604, AG4702, AG4703, AG4901, A4922, AG5401, A5547, AG5602, AG5702, A5704, AG5801, AG5901, A5944, A5959, AG6101, AJW260000R, FPG26932, QR4459 and QP4544 (Asgrow Seeds, Des Moines, Iowa, USA); DKB26-52, DKB28-51, DKB32-52, DKB08-51, DKB09-53, DKB10-52, DKB18-51, DKB26-53, DKB29-51, DKB42-51, DKB35-51 DKB34-51, DKB36-52, DKB37-51, DKB38-52, DKB46-51, DKB54-52 and DeKalb variety CX445 (DeKalb, Ill., USA); 91B91, 92B24, 92B37, 92B63, 92B71, 92B74, 92B75, 92B91, 93B01, 93B11, 93B26, 93B34, 93B35, 93B41, 93B45, 93B51, 93B53, 93B66, 93B81, 93B82, 93B84, 94B01, 94B32, 94B53, 94M80 RR, 94M50 RR, 95B71, 95B95, 95M81 RR, 95M50 RR, 95M30 RR, 9306, 9294, 93M50, 93M93, 94B73, 94B74, 94M41, 94M70, 94M90, 95B32, 95B42, 95B43 and 9344 (Pioneer Hi-bred International, Johnston, Iowa, USA); SSC-251RR, SSC-273CNRR, AGRA 5429RR, SSC-314RR, SSC-315RR, SSC-311STS, SSC-320RR, AGRA5432RR, SSC-345RR, SSC-356RR, SSC-366, SSC-373RR and AGRA5537CNRR (Schlessman Seed Company, Milan, Ohio, USA); 39-E9, 44-R4, 44-R5, 47-G7, 49-P9, 52-Q2, 53-K3, 5646, 58-V8, ARX A48104, ARX B48104, ARX B55104 and GP530 (Armor Beans, Fisher, Ark., USA); HT322STS, HT3596STS, L0332, L0717, L1309CN, L1817, L1913CN, L1984, L2303CN, L2495, L2509CN, L2719CN, L3997CN, L4317CN, RC1303, RC1620, RC1799, RC1802, RC1900, RC1919, RC2020, RC2300, RC2389, RC2424, RC2462, RC2500, RC2504, RC2525, RC2702, RC2964, RC3212, RC3335, RC3354, RC3422, RC3624, RC3636, RC3732, RC3838, RC3864, RC3939, RC3942, RC3964, RC4013, RC4104, RC4233, RC4432, RC4444, RC4464, RC4842, RC4848, RC4992, RC5003, RC5222, RC5332, RC5454, RC5555, RC5892, RC5972, RC6767, RC7402, RT0032, RT0041, RT0065, RT0073, RT0079, RT0255, RT0269, RT0273, RT0312, RT0374, RT0396, RT0476, RT0574, RT0583, RT0662, RT0669, RT0676, RT0684, RT0755, RT0874, RT0907, RT0929, RT0994, RT0995, RT1004, RT1183, RT1199, RT1234, RT1399, RT1413, RT1535, RT1606, RT1741, RT1789, RT1992, RT2000, RT2041, RT2089, RT2092, RT2112, RT2127, RT2200, RT2292, RT2341, RT2430, RT2440, RT2512, RT2544, RT2629, RT2678, RT2732, RT2800, RT2802, RT2822, RT2898, RT2963, RT3176, RT3200, RT3253, RT3432, RT3595, RT3836, RT4098, RX2540, RX2944, RX3444 and TS466RR (Croplan Genetics, Clinton, Ky., USA); 4340RR, 4630RR, 4840RR, 4860RR, 4960RR, 4970RR, 5260RR, 5460RR, 5555RR, 5630RR and 5702RR (Delta Grow, England, Ark., USA); DK3964RR, DK3968RR, DK4461RR, DK4763RR, DK4868RR, DK4967RR, DK5161RR, DK5366RR, DK5465RR, DK55T6, DK5668RR, DK5767RR, DK5967RR, DKXTJ446, DKXTJ448, DKXTJ541, DKXTJ542, DKXTJ543, DKXTJ546, DKXTJ548, DKXTJ549, DKXTJ54J9, DKXTJ54X9, DKXTJ554, DKXTJ555, DKXTJ55J5 and DKXTJ5K57 (Delta King Seed Company, McCrory, Ark., USA); DP 3861RR, DP 4331 RR, DP 4546RR, DP 4724 RR, DP 4933 RR, DP 5414RR, DP 5634 RR, DP 5915 RR, DPX 3950RR, DPX 4891RR, DPX 5808RR (Delta & Pine Land Company, Lubbock, Tex., USA); DG31T31, DG32C38, DG3362NRR, DG3390NRR, DG33A37, DG33B52, DG3443NRR, DG3463NRR, DG3481NRR, DG3484NRR, DG3535NRR, DG3562NRR, DG3583NRR, DG35B40, DG35D33, DG36M49, DG37N43, DG38K57, DG38T47, SX04334, SX04453 (Dyna-gro line, UAP-MidSouth, Cordova, Tenn., USA); 8374RR CYSTX, 8390 NNRR, 8416RR, 8492NRR and 8499NRR (Excel Brand, Camp Point, Ill., USA); 4922RR, 5033RR, 5225RR and 5663RR (FFR Seed, Southhaven, Miss., USA); 3624RR/N, 3824RR/N, 4212RR/N, 4612RR/N, 5012RR/N, 5212RR/N and 5412RR/STS/N (Garst Seed Company, Slater, Iowa, USA); 471, 4R451, 4R485, 4R495, 4RS421 and 5R531 (Gateway Seed Company, Nashville, Ill., USA); H-3606RR, H-3945RR, H-4368RR, H-4749RR, H-5053RR and H-5492RR (Golden Harvest Seeds, Inc., Pekin, Ill., USA); HBK 5324, HBK 5524, HBK R4023, HBK R4623, HBK R4724, HBK R4820, HBK R4924, HBK R4945CX, HBK R5620 and HBK R5624 (Hornbeck Seed Co. Inc., DeWitt, Ark., USA); 341 RR/SCN, 343 RR/SCN, 346 RR/SCN, 349 RR, 355 RR/SCN, 363 RR/SCN, 373 RR, 375 RR, 379 RR/SCN, 379+RR/SCN, 380 RR/SCN, 380+RR/SCN, 381 RR/SCN, 389 RR/SCN, 389+RR/SCN, 393 RR/SCN, 393+RR/SCN, 398 RR, 402 RR/SCN, 404 RR, 424 RR, 434 RR/SCN and 442 RR/SCN (Kruger Seed Company, Dike, Iowa, USA); 3566, 3715, 3875, 3944, 4010 and 4106 (Lewis Hybrids, Inc., Ursa, Ill., USA); C3999NRR (LG Seeds, Elmwood, Ill., USA); Atlanta 543, Austin RR, Cleveland VIIRR, Dallas RR, Denver RRSTS, Everest RR, Grant 3RR, Olympus RR, Phoenix IIIRR, Rocky RR, Rushmore 553RR and Washington IXRR (Merschman Seed Inc., West Point, Iowa, USA); RT 3304N, RT 3603N, RT 3644N, RT 3712N, RT 3804N, RT 3883N, RT 3991N, RT 4044N, RT 4114N, RT 4124N, RT 4201N, RT 4334N, RT 4402N, RT 4480N, RT 4503N, RT 4683N, RT 4993N, RT 5043N, RT 5204, RT 5553N, RT 5773, RT4731N and RTS 4824N (MFA Inc., Columbia, Mo., USA); 9A373NRR, 9A375XRR, 9A385NRS, 9A402NRR, 9A455NRR, 9A485XRR and 9B445NRS (Midland Genetics Group L.L.C., Ottawa, Kans., USA); 3605nRR, 3805nRR, 3903nRR, 3905nRR, 4305nRR, 4404nRR, 4705nRR, 4805nRR, 4904nRR, 4905nRR, 5504nRR and 5505nRR (Midwest Premium Genetics, Concordia, Mo., USA); S37-N4, S39-K6, S40-R9, S42-P7, S43-B1, S49-Q9, S50-N3, S52-U3 and S56-D7 (Syngenta Seeds, Henderson, Ky., USA); NT-3707 RR, NT-3737 RR/SCN, NT-3737+RR/SCN, NT-3737sc RR/SCN, NT-3777+RR, NT-3787 RR/SCN, NT-3828 RR, NT-3839 RR, NT-3909 RR/SCN/STS, NT-3909+RR/SCN/ST, NT-3909sc RR/SCN/S, NT-3919 RR, NT-3922 RR/SCN, NT-3929 RR/SCN, NT-3999 RR/SCN, NT-3999+RR/SCN, NT-3999sc RR/SCN, NT-4040 RR/SCN, NT-4040+RR/SCN, NT-4044 RR/SCN, NT-4122 RR/SCN, NT-4414 RR/SCN/STS, NT-4646 RR/SCN and NT-4747 RR/SCN (NuTech Seed Co., Ames, Iowa, USA); PB-3494NRR, PB-3732RR, PB-3894NRR, PB-3921NRR, PB-4023NRR, PB-4394NRR, PB-4483NRR and PB-5083NRR (Prairie Brand Seed Co., Story City, Iowa, USA); 3900RR, 4401RR, 4703RR, 4860RR, 4910, 4949RR, 5250RR, 5404RR, 5503RR, 5660RR, 5703RR, 5770, 5822RR, PGY 4304RR, PGY 4604RR, PGY 4804RR, PGY 5622RR and PGY 5714RR (Progeny Ag Products, Wynne, Ark., USA); R3595RCX, R3684Rcn, R3814RR, R4095Rcn, R4385Rcn and R4695Rcn (Renze Hybrids Inc., Carroll, Iowa, USA); S3532-4, S3600-4, S3832-4, S3932-4, S3942-4, S4102-4, S4542-4 and S4842-4 (Stine Seed Co., Adel, Iowa USA); 374RR, 398RRS (Taylor Seed Farms Inc., White Cloud, Kans., USA); USG 5002T, USG 510nRR, USG 5601T, USG 7440nRR, USG 7443nRR, USG 7473nRR, USG 7482nRR, USG 7484nRR, USG 7499nRR, USG 7504nRR, USG 7514nRR, USG 7523nRR, USG 7553nRS and USG 7563nRR (UniSouth Genetics Inc., Nashville, Tenn., USA); V38N5RS, V39N4RR, V42N3RR, V48N5RR, V284RR, V28N5RR, V315RR, V35N4RR, V36N5RR, V37N3RR, V40N3RR, V47N3RR, and V562NRR (Royster-Clark Inc., Washington C.H., Ohio, USA); RR2383N, 2525NA, RR2335N, RR2354N, RR2355N, RR2362, RR2385N, RR2392N, RR2392NA, RR2393N, RR2432N, RR2432NA, RR2445N, RR2474N, RR2484N, RR2495N and RR2525N (Willcross Seed, King City Seed, King City, Mo., USA); 1493RR, 1991NRR, 2217RR, 2301NRR, 2319RR, 2321NRR, 2341NRR, 2531NRR, 2541NRR, 2574RR, 2659RR, 2663RR, 2665NRR, 2671NRR, 2678RR, 2685RR, 2765NRR, 2782NRR, 2788NRR, 2791NRR, 3410RR, 3411NRR, 3419NRR, 3421NRR, 3425NRR, 3453NRR, 3461NRR, 3470CRR, 3471NRR, 3473NRR, 3475RR, 3479NRR, 3491NRR, 3499NRR, WX134, WX137, WX177, and WX300 (Wilken Seeds, Pontiac, Ill., USA). An elite plant is a representative plant from an elite line.

Plants of the present disclosure can be part of or generated from a breeding program, or subject to further breeding. The choice of breeding method depends on the mode of plant reproduction, the heritability of the trait(s) being improved, and the type of cultivar used commercially (e.g., F₁ hybrid cultivar, pureline cultivar, etc). Selected, non-limiting approaches, for breeding the plants of the present disclosure are set forth below. A breeding program can be enhanced using marker-assisted selection of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new cultivars requires the development and selection of varieties, the crossing of these varieties and the selection of superior hybrid crosses. The hybrid seed can be produced by manual crosses between selected male-fertile parents or by using male sterility systems. Hybrids are selected for certain single gene traits such as pod color, flower color, seed yield, pubescence color, or herbicide resistance, which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits are crossed to produce an F₁. A F₂ population is produced by selfing one or several F₁'s. Selection of the best individuals from the best families is carried out. Replicated testing of families can begin in the F₄ generation to improve the effectiveness of selection for traits with low heritability. At an advanced stage of inbreeding (e.g., F₆ and F₇), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is faster to thresh pods with a machine than to remove one seed from each by hand for the single-seed procedure. The multiple-seed procedure also makes it possible to plant the same number of seed of a population each generation of inbreeding.

A transgenic plant of the present disclosure may also be reproduced using apomixis. Apomixis is a genetically-controlled method of reproduction in plants where the embryo is formed without union of an egg and a sperm. There are three basic types of apomictic reproduction: 1) apospory where the embryo develops from a chromosomally unreduced egg in an embryo sac derived from the nucleus, 2) diplospory where the embryo develops from an unreduced egg in an embryo sac derived from the megaspore mother cell, and 3) adventitious embryony where the embryo develops directly from a somatic cell. In most forms of apomixis, pseudogamy or fertilization of the polar nuclei to produce endosperm is necessary for seed viability. In apospory, a nurse cultivar can be used as a pollen source for endosperm formation in seeds. The nurse cultivar does not affect the genetics of the aposporous apomictic cultivar since the unreduced egg of the cultivar develops parthenogenetically, but makes possible endosperm production. Apomixis is economically important, especially in transgenic plants, because it causes any genotype, no matter how heterozygous, to breed true. Thus, with apomictic reproduction, heterozygous transgenic plants can maintain their genetic fidelity throughout repeated life cycles. Methods for the production of apomictic plants are known in the art. See, e.g., U.S. Pat. No. 5,811,636.

Transgenic plants comprising or derived from plant cells of this disclosure transformed with recombinant DNA can be further enhanced with stacked traits, e.g., a crop plant having an enhanced trait resulting from expression of DNA disclosed herein in combination with herbicide and/or pest resistance traits. For example, genes of the current disclosure can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present disclosure can be applied include, but are not limited to, glyphosate, dicamba, glufosinate, sulfonylurea, bromoxynil, and norflurazon herbicides.

This disclosure also envisages expressing a plurality of exogenous polynucleotides in a single plant to thereby achieve superior effect on multiple traits, for example, nitrogen use efficiency, biotic or abiotic stress tolerance, yield, vigor, and biomass. Expressing a plurality of exogenous polynucleotides in a single plant can be effected by co-introducing multiple nucleic acid constructs, each including a different exogenous polynucleotide, into a single plant cell. The transformed cell can then be regenerated into a mature plant using the methods described hereinabove. Alternatively, expressing a plurality of exogenous polynucleotides in a single host plant can be effected by co-introducing into a single plant-cell a single nucleic-acid construct including a plurality of different exogenous polynucleotides. Such a construct can be designed with a single promoter sequence which can transcribe a polycistronic messenger RNA including all the different exogenous polynucleotide sequences. Alternatively, the construct can include several promoter sequences each linked to a different exogenous polynucleotide sequence.

Alternatively, expressing a plurality of exogenous polynucleotides can be effected by introducing different nucleic acid constructs, including different exogenous polynucleotides, into a plurality of plants. The regenerated transformed plants can then be cross-bred and the resultant progeny selected for superior yield or fiber traits as described above, using conventional plant breeding techniques.

In one aspect, a plant expressing the exogenous polynucleotide(s) disclosed herein is grown under stress (nitrogen or abiotic) or normal conditions (e.g., biotic conditions and/or conditions with sufficient water, and nutrients such as nitrogen and fertilizer). Such conditions, which depend on the plant being grown, are known to those skilled in the art of agriculture, and are further described above. The instant disclosure also contemplates a method of growing a plant expressing the exogenous polynucleotide(s) disclosed herein under abiotic stress or nitrogen-limiting conditions. Non-limiting examples of abiotic stress conditions include water deprivation, drought, excess of water (e.g., flood, waterlogging), freezing, low temperature, high temperature, strong winds, heavy metal toxicity, anaerobiosis, nutrient deficiency, nutrient excess, salinity, atmospheric pollution, intense light, insufficient light, UV irradiation, etiolation, and atmospheric pollution.

Methods of determining the level in a plant of an exogenous polynucleotide disclosed herein are well known in the art and include, for example, Northern blot analysis, reverse transcription polymerase chain reaction (RT-PCR) analysis (including quantitative, semi-quantitative, or real-time RT-PCR), and RNA-in situ hybridization.

Plants exogenously expressing the polynucleotide of the disclosure can be screened to identify those that show the greatest increase of a desired plant trait. In one aspect, the present disclosure also provides a method of evaluating a trait of a plant, the method comprising: (a) expressing in a plant or a portion thereof the nucleic acid construct and (b) evaluating a trait of a plant as compared to a wild type plant of the same type, thereby evaluating the trait of the plant.

The effect of a transgene or an exogenous polynucleotide on different plant characteristics may be determined by any method known to one of ordinary skill in the art.

Tolerance to abiotic stress (e.g., tolerance to drought or salinity) can be evaluated by determining the differences in physiological and/or physical condition, including but not limited to, vigor, growth, size, or root length, or specifically, leaf color or leaf area size of the transgenic plant compared to a non-modified plant of the same species grown under the same conditions. Other techniques for evaluating tolerance to abiotic stress include, but are not limited to, measuring chlorophyll fluorescence, photosynthetic rates, and gas exchange rates. Further assays for evaluating tolerance to abiotic stress are provided herein below and in the Examples section which follows.

Drought Tolerance Assay—

Soil-based drought screens are performed with plants overexpressing the polynucleotides detailed above. Seeds from control Arabidopsis plants, or other transgenic plants overexpressing nucleic acid of the disclosure are germinated and transferred to pots. Drought stress is obtained after irrigation is ceased. Transgenic and control plants are compared to each other when the majority of the control plants develop severe wilting. Plants are re-watered after obtaining a significant fraction of the control plants displaying a severe wilting. Plants are ranked comparing to controls for each of two criteria: tolerance to the drought conditions and recovery (survival) following re-watering.

Quantitative parameters of tolerance measured include, but are not limited to, the average wet and dry weight, growth rate, leaf size, leaf coverage (overall leaf area), the weight of the seeds yielded, the average seed size, and the number of seeds produced per plant. Transformed plants not exhibiting substantial physiological and/or morphological effects, or exhibiting higher biomass than wild-type plants, are identified as drought stress tolerant plants.

Salinity Tolerance Assay—

Transgenic plants with tolerance to high salt concentrations are expected to exhibit better germination, seedling vigor, or growth in high salt. Salt stress can be effected in many ways such as, for example, by irrigating the plants with a hyperosmotic solution, by cultivating the plants hydroponically in a hyperosmotic growth solution (e.g., Hoagland solution with added salt), or by culturing the plants in a hyperosmotic growth medium (e.g., 50% Murashige-Skoog medium (MS medium) with added salt). Since different plants vary considerably in their tolerance to salinity, the salt concentration in the irrigation water, growth solution, or growth medium can be adjusted according to the specific characteristics of the specific plant cultivar or variety, so as to inflict a mild or moderate effect on the physiology and/or morphology of the plants (for guidelines as to appropriate concentration, see Bernstein and Kafkafi, Root Growth Under Salinity Stress In: Plant Roots, The Hidden Half, 3rd ed., Waisel Y, Eshel A and Kafkafi U. (eds.), Marcel Dekker Inc., New York, 2002, and references therein).

For example, a salinity tolerance test can be performed by irrigating plants at different developmental stages with increasing concentrations of sodium chloride (for example, 50 mM, 150 mM, 300 mM NaCl) applied from the bottom and from above to ensure even dispersal of salt. Following exposure to the stress condition, the plants are frequently monitored until substantial physiological and/or morphological effects appear in wild-type plants. Thus, the external phenotypic appearance, degree of chlorosis, and overall success to reach maturity and yield progeny are compared between control and transgenic plants. Quantitative parameters of tolerance measured include, but are not limited to, the average wet and dry weight, growth rate, leaf size, leaf coverage (overall leaf area), the weight of the seeds yielded, the average seed size, and the number of seeds produced per plant. Transformed plants not exhibiting substantial physiological and/or morphological effects, or exhibiting higher biomass than wild-type plants, are identified as abiotic stress tolerant plants.

Osmotic Tolerance Test—

Osmotic stress assays (including sodium chloride and PEG assays) are conducted to determine if an osmotic stress phenotype was sodium chloride-specific or if it was a general osmotic-stress-related phenotype. Plants which are tolerant to osmotic stress may have more tolerance to drought and/or freezing. For salt and osmotic stress experiments, the medium is supplemented for example with 50 mM, 100 mM, 200 mM NaCl, or 15%, 20% or 25% PEG.

Cold Stress Tolerance—

One way to analyze cold stress is as follows. Mature (25 day old) plants are transferred to 4° C. chambers for 1 or 2 weeks, with constitutive light. Later on, plants are moved back to the greenhouse. Two weeks later, damages from a chilling period, resulting in growth retardation and other phenotypes, are compared between control and transgenic plants, by measuring plant weight (wet and dry), and by comparing growth rates measured as time to flowering, plant size, yield, and the like.

Heat Stress Tolerance—

One way to measure heat stress tolerance is by exposing the plants to temperatures above 34° C. for a certain period. Plant tolerance is examined after transferring the plants back to 22° C. for recovery and evaluation after 5 days relative to internal controls (non-transgenic plants) or plants not exposed to either cold or heat stress.

The biomass, vigor, and yield of the plant can also be evaluated using any method known to one of ordinary skill in the art. Thus, for example, plant vigor can be calculated by the increase in growth parameters such as leaf area, fiber length, rosette diameter, plant fresh weight, and the like, per time.

As mentioned, the increase of plant yield can be determined by various parameters. For example, increased yield of rice may be manifested by an increase in one or more of the following: number of plants per growing area, number of panicles per plant, number of spikelets per panicle, number of flowers per panicle, seed filling rate, thousand kernel weight (1000-weight), oil content per seed, and starch content per seed, among others. An increase in yield may also result in modified architecture, or may occur because of modified architecture. Similarly, increased yield of soybean may be manifested by an increase in one or more of the following: number of plants per growing area, number of pods per plant, number of seeds per pod, seed filling rate, thousand seed weight (1000-weight), oil content per seed, and protein content per seed, among others. Alternatively, an increase in yield of soybean may also be manifested by a reduction of pod shattering. An increase in yield may also result in modified architecture, or may occur because of modified architecture.

In an aspect, a transgenic plant of the present disclosure can show enhanced performance based on one or more of the assays set forth herein, including without limitation, in the Examples. In an aspect, a transgenic plant of the present disclosure can be a homozygous plant showing enhanced performance based on one or more of the assays set forth herein, including without limitation, in the Examples. In an aspect, a transgenic plant of the present disclosure can be a hybrid plant showing enhanced performance based on one or more of the assays set forth herein, including without limitation, in the Examples. In an aspect, a transgenic plant of the present disclosure can be a heterozygous plant showing enhanced performance based on one or more of the assays set forth herein, including without limitation, in the Examples.

In an aspect, a transgenic plant of the present disclosure can exhibit transiently or constitutively one or more traits or phenotypes selected from an enhanced trait, including without limitation, abiotic stress tolerance, improved drought tolerance, increased biomass, improved vigor, improved yield, enhanced water use efficiency, increased root branching, increased root depth, increased salt tolerance, resistance to heat shock damage, and improved germination.

In an aspect, a transgenic plant of the present disclosure can be a homozygous plant that exhibits transiently or constitutively one or more traits or phenotypes selected from an enhanced trait, including without limitation, abiotic stress tolerance, improved drought tolerance, increased biomass, improved vigor, improved yield, enhanced water use efficiency, increased root branching, increased root depth, increased salt tolerance, resistance to heat shock damage, and improved germination.

In an aspect, a transgenic plant of the present disclosure can be a hybrid plant that exhibits transiently or constitutively one or more traits or phenotypes selected from an enhanced trait, including without limitation, abiotic stress tolerance, improved drought tolerance, increased biomass, improved vigor, improved yield, enhanced water use efficiency, increased root branching, increased root depth, increased salt tolerance, resistance to heat shock damage, and improved germination.

In an aspect, a transgenic plant of the present disclosure can be a heterozygous plant that exhibits transiently or constitutively one or more traits or phenotypes selected from an enhanced trait, including without limitation, abiotic stress tolerance, improved drought tolerance, increased biomass, improved vigor, improved yield, enhanced water use efficiency, increased root branching, increased root depth, increased salt tolerance, resistance to heat shock damage, and improved germination.

The following Examples are presented for the purposes of illustration and should not be construed as limitations.

### EXAMPLES

### Example 1

### Differential Expression of miRNAs in Soybean Plant Under Abiotic Stress Versus Optimal Conditions

### Plant Material

Soybean seeds are obtained from Taam-Teva shop (Israel). Plants are grown at 28° C. under a 16 hours light: 8 hours dark regime.

### Stress Induction

Plants are grown under standard conditions as described above until seedlings are two weeks old. Next, plants are divided into two groups: control plants are irrigated with tap water twice a week and drought-treated plants receive no irrigation. The experiment continues for one week, after which plants are harvested for RNA extraction.

### Total RNA Extraction

Total RNA of leaf samples from eight biological repeats are extracted using the mirVana™ kit (Ambion, Austin, Tex.) by pooling 3-4 plants to one biological repeat.

### Microarray Design

Custom microarrays are manufactured by Agilent Technologies by in situ synthesis of DNA oligonucleotide probes for 890 plant and algal microRNAs, with each probe being printed in triplicate.

### Results

The following table presents sequences that are found to be differentially expressed in soybean grown under various drought or control conditions. Upregulated means the sequence is induced under irrigation limiting conditions (drought) and downregulated means the sequence is repressed under irrigation limiting conditions.

TABLE 1
Differentially Expressed Small RNAs in Soybean Plants
Growing under Drought versus Optimal Conditions.

| | | SEQ ID | Direction | | |
|---|---|---|---|---|---|
| | | NO | of miRNA | | |
| | SEQ ID | Stem- | Expression | | |
| | NO of | Loop | Change | | |
| | Mature | Precursor | under | Fold | |
| Mir Name | Sequence | Sequence | Drought | Change | P value |
| gma-miR164 | 1 | 6 | Up | 1.81 | 2.90E−03 |
| gma-miR168 | 2 | 7 | Up | 1.67 | 1.00E−05 |
| osa-miR397b | 3 | 8 | Down | 3.09 | 6.70E−03 |
| sof-miR408e | 4 | 9 | Down | 1.74 | 1.80E−02 |
| smo- | 5 | 10 | Down | 1.78 | 4.60E−04 |
| miR1093 | | | | | |

### Example 2

### Assessments of Abiotic Stress Tolerance in Control and Transgenic Plants

Transgenic plants with tolerance to abiotic stress in the form of extreme deficiency in water are expected to exhibit better overall survival and growth compared to control non-transgenic plants. Since different plants vary considerably in their tolerance to drought stress, the duration of drought effected can be tailored to the specific plant cultivar or variety (for guidelines specifically to appropriate salt concentrations see, Bernstein and Kafkafi, Root Growth Under Salinity Stress In: Plant Roots, The Hidden Half 3^{rd}, ed. Waisel Y, Eshel A and Kafkafi U. (eds.), Marcel Dekker Inc., New York, 2002).

Quantitative parameters of tolerance measured include, but are not limited to, the average wet and dry weight, growth rate, leaf size, leaf coverage (overall leaf area), the weight of the seeds yielded, the average seed size, and the number of seeds produced per plant. Under normal conditions, transgenic plants are expected to exhibit a phenotype equivalent or superior to that of the wild type plants. Following stress induction, transformed plants not exhibiting substantial physiological and/or morphological effects, or exhibiting higher biomass than wild-type plants, are identified as abiotic stress tolerant plants.

### 2.1 Methods for Drought Tolerance Assessment

### 2.1.1. Soil-Based Drought Tolerance Assay

Screens are performed with plants over-expressing the differential small RNAs detailed above. Briefly, seeds from control Arabidopsis plants, or other transgenic plants over-expressing the small RNA molecule of the disclosure are germinated and transferred to pots. Drought stress is obtained after irrigation is ceased and the two plant types (transgenic and control plants) are compared when most control plants develop severe wilting, and concurrently, rehydration of the plants is initiated. Transgenic plants are ranked on two levels compared to controls: (1) tolerance to drought conditions and (2) recovery (survival) following re-watering.

To illustrate and elaborate on the above drought tolerance assays of any given wild type plant compared to a corresponding transgenic plant (in which a drought-associated miRNA has been over-expressed), two different approaches are taken as follows:

Lethal drought stress—whereby wild type (used as a control) and transgenic plants (1-3 weeks old) are grown under prolonged extreme drought conditions (duration varies in accordance with plant species). Next, a recovery attempt is implemented during which plants are regularly irrigated and survival level is estimated in the two plant groups 1-2 days post irrigation initiation. While the control (wild type) plant is not expected to survive this extreme stress, the transgenic plant is expected to demonstrate some improved drought tolerance, usually within hours of re-hydration.

Non-lethal drought stress—whereby wild type (used as a control) and transgenic plants (1-3 weeks old) are grown under regular short-term cycles of drought and re-hydration steps, such that re-hydration is applied when general visible drought symptoms (e.g., evident decrease in turgor pressure of lower leaves) emerge in the experimental plants. This drought/irrigation alternating treatment continues until the flowering stage of the plants is reached, followed by an evaluation of dry matter weight. Both wild-type and transgenic plants are expected to survive this non-lethal stress; however, measurable differences in drought tolerance are demonstrated by increased yield of the transgenic compared with the wild type plants.

### 2.1.2. Drought Tolerance Assay Using Sorbitol

Another assay designed to assess whether transgenic plants are more tolerant to drought or severe water deprivation compared to control plants involves induction of an osmotic stress by the non-ionic osmolyte sorbitol. Control and transgenic plants are germinated and grown in plant-agar plates for 4 days, after which they are transferred to plates containing 500 mM sorbitol, to cause delayed growth. Following the stress treatment, control and transgenic plants are compared by measuring plant weight (wet and dry), yield, and by growth rates measured as time to flowering.

### 2.2 Methods for Salinity Tolerance Assessment

Osmotic stress assays, such as chloride and mannitol assays, are aimed to determine whether an osmotic stress phenotype is sodium chloride-specific or a result of a general osmotic stress. Plants which are tolerant to osmotic stress may also exhibit tolerance to drought and/or freezing. For salt and osmotic stress germination experiments, the medium is supplemented with 50, 100, or 200 mM NaCl or 100 mM, 200 mM NaCl, 400 mM mannitol.

### 2.3 Methods for Heat Stress Tolerance Assessment

Heat stress tolerance is achieved by exposing the plants to temperatures above 34° C. for a certain period. Plant tolerance is examined after transferring the plants back to 22° C. for recovery and evaluation after 5 days relative to internal controls (non-transgenic plants) or plants not exposed to either cold or heat stress.

### 2.4 Methods for Cold Stress Tolerance Assessment

To analyze cold stress, mature (25 day old) plants are transferred to 4° C. chambers for 1 or 2 weeks, with constitutive light. Next, plants are moved back to the greenhouse for 2 weeks to recover. Following the recovery period, chilling damages such as growth retardation are determined based on measurements of plant weight (wet and dry) and growth rates (e.g. time to flowering, plant size, yield, etc.) taken on control and transgenic plants.

### Example 3

### Identification of Homologous and Orthologous Sequences of Differential Small RNAs Associated with Enhanced Abiotic Stress Tolerance

The small RNA sequences of the disclosure that were either down- or up-regulated under abiotic stress conditions were examined for homologous and orthologous sequences using the miRBase database (available on the internet at www.rmirbase.org) and the Plant MicroRNA Database (PMRD, available on the internet at bioinformatics.cau.edu.cn/PMRD). The mature miRNA sequences that are homologous or orthologous to the miRNAs of the disclosure (listed in Table 1) are found using miRNA public databases, having at least 80% identity of the entire small RNA length, and are summarized in Table 2 below.

TABLE 2
Summary of Homologs/Orthologs of Small RNA Probes of Table 1.

| | | | | | | | | SEQ ID |
|---|---|---|---|---|---|---|---|---|
| | | | | | | | | NO of |
| | SEQ ID | | SEQ ID | | SEQ ID | | Percentage | stem-loop |
| Query | NO of | | NO of | | NO of | | of | sequence |
| Small | Mature | Query | Stem- | Homolog | homolog | Homolog | Identity | of |
| RNA | miRNA | miRNA | loop | miRNA | miRNA | miRNA | (1 = | homolog |
| Name | Sequence | length | sequence | Name | sequence | length | 100%) | miRNA |
| Gma- | 1 | 21 | 6 | aly- | 11 | 21 | 1 | 137 |
| miR164 | | | | miR164a | | | | |
| Gma- | 1 | 21 | 6 | aly- | 12 | 21 | 1 | 138 |
| miR164 | | | | miR164b | | | | |
| Gma- | 1 | 21 | 6 | aly- | 13 | 21 | 0.95 | 139 |
| miR164 | | | | miR164c | | | | |
| Gma- | 1 | 21 | 6 | ath- | 14 | 21 | 1 | 140 |
| miR164 | | | | miR164a | | | | |
| Gma- | 1 | 21 | 6 | ath- | 15 | 21 | 1 | 141 |
| miR164 | | | | miR164b | | | | |
| Gma- | 1 | 21 | 6 | ath- | 16 | 21 | 0.95 | 142 |
| miR164 | | | | miR164c | | | | |
| Gma- | 1 | 21 | 6 | bdi- | 17 | 21 | 1 | 143 |
| miR164 | | | | miR164a | | | | |
| Gma- | 1 | 21 | 6 | bdi- | 18 | 21 | 1 | 144 |
| miR164 | | | | miR164b | | | | |
| Gma- | 1 | 21 | 6 | bdi- | 19 | 21 | 0.95 | 145 |
| miR164 | | | | miR164c | | | | |
| Gma- | 1 | 21 | 6 | bdi- | 20 | 21 | 1 | 146 |
| miR164 | | | | miR164d | | | | |
| Gma- | 1 | 21 | 6 | bdi- | 21 | 21 | 1 | 147 |
| miR164 | | | | miR164e | | | | |
| Gma- | 1 | 21 | 6 | bdi- | 22 | 21 | 0.9 | 148 |
| miR164 | | | | miR164f | | | | |
| Gma- | 1 | 21 | 6 | bna- | 23 | 21 | 1 | 149 |
| miR164 | | | | miR164 | | | | |
| Gma- | 1 | 21 | 6 | bra- | 24 | 21 | 1 | 150 |
| miR164 | | | | miR164a | | | | |
| Gma- | 1 | 21 | 6 | csi- | 25 | 21 | 1 | 151 |
| miR164 | | | | miR164 | | | | |
| Gma- | 1 | 21 | 6 | ctr- | 26 | 21 | 1 | 152 |
| miR164 | | | | miR164 | | | | |
| Gma- | 1 | 21 | 6 | far- | 27 | 21 | 0.9 | 153 |
| miR164 | | | | miR164a | | | | |
| Gma- | 1 | 21 | 6 | far- | 28 | 21 | 0.9 | 154 |
| miR164 | | | | miR164b | | | | |
| Gma- | 1 | 21 | 6 | ghr- | 29 | 21 | 1 | 155 |
| miR164 | | | | miR164 | | | | |
| Gma- | 1 | 21 | 6 | mtr- | 30 | 21 | 1 | 156 |
| miR164 | | | | miR164a | | | | |
| Gma- | 1 | 21 | 6 | mtr- | 31 | 21 | 1 | 157 |
| miR164 | | | | miR164b | | | | |
| Gma- | 1 | 21 | 6 | mtr- | 32 | 21 | 1 | 158 |
| miR164 | | | | miR164c | | | | |
| Gma- | 1 | 21 | 6 | mtr- | 33 | 21 | 0.9 | 159 |
| miR164 | | | | miR164d | | | | |
| Gma- | 1 | 21 | 6 | osa- | 34 | 21 | 1 | 160 |
| miR164 | | | | miR164a | | | | |
| Gma- | 1 | 21 | 6 | osa- | 35 | 21 | 1 | 161 |
| miR164 | | | | miR164b | | | | |
| Gma- | 1 | 21 | 6 | osa- | 36 | 21 | 0.95 | 162 |
| miR164 | | | | miR164c | | | | |
| Gma- | 1 | 21 | 6 | osa- | 37 | 21 | 0.95 | 163 |
| miR164 | | | | miR164d | | | | |
| Gma- | 1 | 21 | 6 | osa- | 38 | 21 | 0.9 | 164 |
| miR164 | | | | miR164e | | | | |
| Gma- | 1 | 21 | 6 | osa- | 39 | 21 | 1 | 165 |
| miR164 | | | | miR164f | | | | |
| Gma- | 1 | 21 | 6 | ptc- | 40 | 21 | 1 | 166 |
| miR164 | | | | miR164a | | | | |
| Gma- | 1 | 21 | 6 | ptc- | 41 | 21 | 1 | 167 |
| miR164 | | | | miR164b | | | | |
| Gma- | 1 | 21 | 6 | ptc- | 42 | 21 | 1 | 168 |
| miR164 | | | | miR164c | | | | |
| Gma- | 1 | 21 | 6 | ptc- | 43 | 21 | 1 | 169 |
| miR164 | | | | miR164d | | | | |
| Gma- | 1 | 21 | 6 | ptc- | 44 | 21 | 1 | 170 |
| miR164 | | | | miR164e | | | | |
| Gma- | 1 | 21 | 6 | ptc- | 45 | 21 | 0.9 | 171 |
| miR164 | | | | miR164f | | | | |
| Gma- | 1 | 21 | 6 | rco- | 46 | 21 | 1 | 172 |
| miR164 | | | | miR164a | | | | |
| Gma- | 1 | 21 | 6 | rco- | 47 | 21 | 1 | 173 |
| miR164 | | | | miR164b | | | | |
| Gma- | 1 | 21 | 6 | rco- | 48 | 21 | 1 | 174 |
| miR164 | | | | miR164c | | | | |
| Gma- | 1 | 21 | 6 | rco- | 49 | 21 | 0.9 | 175 |
| miR164 | | | | miR164d | | | | |
| Gma- | 1 | 21 | 6 | sbi- | 50 | 21 | 1 | 176 |
| miR164 | | | | miR164 | | | | |
| Gma- | 1 | 21 | 6 | sbi- | 51 | 21 | 0.95 | 177 |
| miR164 | | | | miR164b | | | | |
| Gma- | 1 | 21 | 6 | sbi- | 52 | 21 | 0.86 | 178 |
| miR164 | | | | miR164c | | | | |
| Gma- | 1 | 21 | 6 | sbi- | 53 | 21 | 1 | 179 |
| miR164 | | | | miR164d | | | | |
| Gma- | 1 | 21 | 6 | sbi- | 54 | 21 | 1 | 180 |
| miR164 | | | | miR164e | | | | |
| Gma- | 1 | 21 | 6 | tae- | 55 | 21 | 1 | 181 |
| miR164 | | | | miR164 | | | | |
| Gma- | 1 | 21 | 6 | tcc- | 56 | 21 | 1 | 182 |
| miR164 | | | | miR164a | | | | |
| Gma- | 1 | 21 | 6 | tcc- | 57 | 21 | 1 | 183 |
| miR164 | | | | miR164b | | | | |
| Gma- | 1 | 21 | 6 | tcc- | 58 | 21 | 0.9 | 184 |
| miR164 | | | | miR164c | | | | |
| Gma- | 1 | 21 | 6 | vvi- | 59 | 21 | 1 | 185 |
| miR164 | | | | miR164a | | | | |
| Gma- | 1 | 21 | 6 | vvi- | 60 | 21 | 0.9 | 186 |
| miR164 | | | | miR164b | | | | |
| Gma- | 1 | 21 | 6 | vvi- | 61 | 21 | 1 | 187 |
| miR164 | | | | miR164c | | | | |
| Gma- | 1 | 21 | 6 | vvi- | 62 | 21 | 1 | 188 |
| miR164 | | | | miR164d | | | | |
| Gma- | 1 | 21 | 6 | zma- | 63 | 21 | 1 | 189 |
| miR164 | | | | miR164a | | | | |
| Gma- | 1 | 21 | 6 | zma- | 64 | 21 | 1 | 190 |
| miR164 | | | | miR164b | | | | |
| Gma- | 1 | 21 | 6 | zma- | 65 | 21 | 1 | 191 |
| miR164 | | | | miR164c | | | | |
| Gma- | 1 | 21 | 6 | zma- | 66 | 21 | 1 | 192 |
| miR164 | | | | miR164d | | | | |
| Gma- | 1 | 21 | 6 | zma- | 67 | 21 | 0.86 | 193 |
| miR164 | | | | miR164e | | | | |
| Gma- | 1 | 21 | 6 | zma- | 68 | 21 | 0.95 | 194 |
| miR164 | | | | miR164f | | | | |
| Gma- | 1 | 21 | 6 | zma- | 69 | 21 | 1 | 195 |
| miR164 | | | | miR164g | | | | |
| Gma- | 1 | 21 | 6 | zma- | 70 | 21 | 0.9 | 196 |
| miR164 | | | | miR164h | | | | |
| gma- | 2 | 21 | 7 | aly- | 71 | 21 | 1 | 197 |
| miR168 | | | | miR168a | | | | |
| gma- | 2 | 21 | 7 | aly- | 72 | 21 | 1 | 198 |
| miR168 | | | | miR168b | | | | |
| gma- | 2 | 21 | 7 | aqc- | 73 | 21 | 0.81 | 199 |
| miR168 | | | | miR168 | | | | |
| gma- | 2 | 21 | 7 | ath- | 74 | 21 | 1 | 200 |
| miR168 | | | | miR168a | | | | |
| gma- | 2 | 21 | 7 | ath- | 75 | 21 | 1 | 201 |
| miR168 | | | | miR168b | | | | |
| gma- | 2 | 21 | 7 | bdi- | 76 | 21 | 0.9 | 202 |
| miR168 | | | | miR168 | | | | |
| gma- | 2 | 21 | 7 | bna- | 77 | 21 | 1 | 203 |
| miR168 | | | | miR168 | | | | |
| gma- | 2 | 21 | 7 | ccl- | 78 | 21 | 1 | 204 |
| miR168 | | | | miR168 | | | | |
| gma- | 2 | 21 | 7 | crt- | 79 | 21 | 1 | 205 |
| miR168 | | | | miR168 | | | | |
| gma- | 2 | 21 | 7 | hvu- | 80 | 21 | 0.9 | 206 |
| miR168 | | | | miR168- | | | | |
| | | | | 5p | | | | |
| gma- | 2 | 21 | 7 | mtr- | 81 | 21 | 0.9 | 207 |
| miR168 | | | | miR168 | | | | |
| gma- | 2 | 21 | 7 | osa- | 82 | 21 | 0.9 | 208 |
| miR168 | | | | miR168a | | | | |
| gma- | 2 | 21 | 7 | osa- | 83 | 21 | 0.86 | 209 |
| miR168 | | | | miR168b | | | | |
| gma- | 2 | 21 | 7 | ptc- | 84 | 21 | 1 | 210 |
| miR168 | | | | miR168a | | | | |
| gma- | 2 | 21 | 7 | ptc- | 85 | 21 | 1 | 211 |
| miR168 | | | | miR168b | | | | |
| gma- | 2 | 21 | 7 | rco- | 86 | 21 | 1 | 212 |
| miR168 | | | | miR168 | | | | |
| gma- | 2 | 21 | 7 | sbi- | 87 | 21 | 0.9 | 213 |
| miR168 | | | | miR168 | | | | |
| gma- | 2 | 21 | 7 | sof- | 88 | 21 | 0.9 | 214 |
| miR168 | | | | miR168a | | | | |
| gma- | 2 | 21 | 7 | sof- | 89 | 20 | 0.86 | 215 |
| miR168 | | | | miR168b | | | | |
| gma- | 2 | 21 | 7 | ssp- | 90 | 21 | 0.9 | 216 |
| miR168 | | | | miR168a | | | | |
| gma- | 2 | 21 | 7 | tcc- | 91 | 21 | 1 | 217 |
| miR168 | | | | miR168 | | | | |
| gma- | 2 | 21 | 7 | vvi- | 92 | 21 | 1 | 218 |
| miR168 | | | | miR168 | | | | |
| gma- | 2 | 21 | 7 | zma- | 93 | 21 | 0.9 | 219 |
| miR168 | | | | miR168a | | | | |
| gma- | 2 | 21 | 7 | zma- | 94 | 21 | 0.9 | 220 |
| miR168 | | | | miR168b | | | | |
| osa- | 3 | 21 | 8 | aly- | 95 | 21 | 0.95 | 221 |
| miR397b | | | | miR397a | | | | |
| osa- | 3 | 21 | 8 | ath- | 96 | 21 | 0.95 | 222 |
| miR397b | | | | miR397a | | | | |
| osa- | 3 | 21 | 8 | bdi- | 97 | 21 | 0.95 | 223 |
| miR397b | | | | miR397a | | | | |
| osa- | 3 | 21 | 8 | bdi- | 98 | 21 | 0.9 | 224 |
| miR397b | | | | miR397b | | | | |
| osa- | 3 | 21 | 8 | bna- | 99 | 22 | 0.95 | 225 |
| miR397b | | | | miR397a | | | | |
| osa- | 3 | 21 | 8 | bna- | 100 | 22 | 0.95 | 226 |
| miR397b | | | | miR397b | | | | |
| osa- | 3 | 21 | 8 | csi- | 101 | 21 | 0.95 | 227 |
| miR397b | | | | miR397 | | | | |
| osa- | 3 | 21 | 8 | hvu- | 102 | 21 | 0.86 | 228 |
| miR397b | | | | miR397 | | | | |
| osa- | 3 | 21 | 8 | osa- | 103 | 21 | 0.95 | 229 |
| miR397b | | | | miR397a | | | | |
| osa- | 3 | 21 | 8 | pab- | 104 | 21 | 0.9 | 230 |
| miR397b | | | | miR397 | | | | |
| osa- | 3 | 21 | 8 | ptc- | 105 | 21 | 0.95 | 231 |
| miR397b | | | | miR397a | | | | |
| osa- | 3 | 21 | 8 | ptc- | 106 | 21 | 0.9 | 232 |
| miR397b | | | | miR397b | | | | |
| osa- | 3 | 21 | 8 | ptc- | 107 | 21 | 0.86 | 233 |
| miR397b | | | | miR397c | | | | |
| osa- | 3 | 21 | 8 | rco- | 108 | 21 | 0.95 | 234 |
| miR397b | | | | miR397 | | | | |
| osa- | 3 | 21 | 8 | sbi- | 109 | 21 | 0.95 | 235 |
| miR397b | | | | miR397 | | | | |
| osa- | 3 | 21 | 8 | sly- | 110 | 20 | 0.9 | 236 |
| miR397b | | | | miR397 | | | | |
| osa- | 3 | 21 | 8 | tcc- | 111 | 21 | 0.95 | 237 |
| miR397b | | | | miR397 | | | | |
| osa- | 3 | 21 | 8 | vvi- | 112 | 21 | 0.95 | 238 |
| miR397b | | | | miR397a | | | | |
| osa- | 3 | 21 | 8 | zma- | 113 | 21 | 0.9 | 239 |
| miR397b | | | | miR397a | | | | |
| osa- | 3 | 21 | 8 | zma- | 114 | 21 | 0.9 | 240 |
| miR397b | | | | miR397b | | | | |
| sof- | 4 | 21 | 9 | ahy- | 115 | 21 | 0.9 | 241 |
| miR408e | | | | miR408- | | | | |
| | | | | 3p | | | | |
| sof- | 4 | 21 | 9 | aly- | 116 | 21 | 0.9 | 242 |
| miR408e | | | | miR408 | | | | |
| sof- | 4 | 21 | 9 | ath- | 117 | 21 | 0.9 | 243 |
| miR408e | | | | miR408 | | | | |
| sof- | 4 | 21 | 9 | csi- | 118 | 21 | 0.9 | 244 |
| miR408e | | | | miR408 | | | | |
| sof- | 4 | 21 | 9 | osa- | 119 | 21 | 0.95 | 245 |
| miR408e | | | | miR408 | | | | |
| sof- | 4 | 21 | 9 | ppt- | 120 | 22 | 0.9 | 246 |
| miR408e | | | | miR408 | | | | |
| sof- | 4 | 21 | 9 | ppt- | 121 | 21 | 0.9 | 247 |
| miR408e | | | | miR408b | | | | |
| sof- | 4 | 21 | 9 | pta- | 122 | 21 | 0.9 | 248 |
| miR408e | | | | miR408 | | | | |
| sof- | 4 | 21 | 9 | ptc- | 123 | 21 | 0.9 | 249 |
| miR408e | | | | miR408 | | | | |
| sof- | 4 | 21 | 9 | rco- | 124 | 21 | 0.95 | 250 |
| miR408e | | | | miR408 | | | | |
| sof- | 4 | 21 | 9 | sbi- | 125 | 21 | 0.95 | 251 |
| miR408e | | | | miR408 | | | | |
| sof- | 4 | 21 | 9 | smo- | 126 | 22 | 0.9 | 252 |
| miR408e | | | | miR408 | | | | |
| sof- | 4 | 21 | 9 | sof- | 127 | 21 | 0.95 | 253 |
| miR408e | | | | miR408a | | | | |
| sof- | 4 | 21 | 9 | sof- | 128 | 21 | 0.95 | 254 |
| miR408e | | | | miR408b | | | | |
| sof- | 4 | 21 | 9 | sof- | 129 | 21 | 0.95 | 255 |
| miR408e | | | | miR408c | | | | |
| sof- | 4 | 21 | 9 | sof- | 130 | 21 | 0.95 | 256 |
| miR408e | | | | miR408d | | | | |
| sof- | 4 | 21 | 9 | ssp- | 131 | 21 | 0.95 | 257 |
| miR408e | | | | miR408a | | | | |
| sof- | 4 | 21 | 9 | ssp- | 132 | 21 | 0.95 | 258 |
| miR408e | | | | miR408d | | | | |
| sof- | 4 | 21 | 9 | tae- | 133 | 21 | 0.95 | 259 |
| miR408e | | | | miR408 | | | | |
| sof- | 4 | 21 | 9 | vvi- | 134 | 21 | 0.9 | 260 |
| miR408e | | | | miR408 | | | | |
| sof- | 4 | 21 | 9 | zma- | 135 | 21 | 0.95 | 261 |
| miR408e | | | | miR408 | | | | |
| sof- | 4 | 21 | 9 | zma- | 136 | 21 | 0.95 | 262 |
| miR408e | | | | miR408b | | | | |

### Example 4

### Identification of miRNAs Associated with Abiotic Stress and Target Prediction Using Bioinformatics Tools

Small RNAs that are potentially associated with improved abiotic or biotic stress tolerance can be identified by proprietary computational algorithms that analyze RNA expression profiles alongside publicly-available gene and protein databases. A high throughput screening is performed on microarrays loaded with miRNAs that were found to be differential under multiple stress and optimal environmental conditions and in different plant tissues. The initial trait-associated miRNAs are later validated by quantitative Real Time PCR (qRT-PCR).

Target prediction—homologous or orthologous genes to the genes of interest in soybean are found through a proprietary tool that analyzes publicly-available genomic, as well as expression and gene annotation, databases from multiple plant species. Homologous and orthologous protein and nucleotide sequences of target genes of the small RNA sequences of the disclosure, were found using BLAST having at least 70% identity on at least 60% of the entire master gene length, and are summarized in Table 3 below.

TABLE 3
Target Genes of Small RNA Molecules Associated with Abiotic Stress
Tolerance in Soybean Plants.

| | miRNA | | | | Nucleotide |
|---|---|---|---|---|---|
| miRNA | Binding | Homolog NCBI | | Protein SEQ | SEQ ID |
| Name | Position | Accession | Organism | ID NO | NO |
| gma-miR164 | 719-739 | AAK84883 | Phaseolus vulgaris | 263 | 317 |
| gma-miR164 | | ACC66316 | Glycine max | 264 | 318 |
| gma-miR164 | | ACD39385 | Glycine max | 265 | 319 |
| gma-miR164 | | ACU24381 | Glycine max | 266 | 320 |
| gma-miR164 | | AEE99077 | Medicago | 267 | 321 |
| | | | truncatula | | |
| gma-miR164 | | XP_002310688 | Populus trichocarpa | 268 | 322 |
| gma-miR164 | | XP_002529954 | Ricinus communis | 269 | 323 |
| gma-miR164 | | XP_002307195 | Populus trichocarpa | 270 | 324 |
| gma-miR164 | 720-740 | ACD39385 | Glycine max | 271 | 325 |
| gma-miR164 | | ACU24381 | Glycine max | 272 | 326 |
| gma-miR164 | | ACC66316 | Glycine max | 273 | 327 |
| gma-miR164 | | AAK84883 | Phaseolus vulgaris | 274 | 328 |
| gma-miR164 | | AEE99077 | Medicago | 275 | 329 |
| | | | truncatula | | |
| gma-miR164 | | XP_002529954 | Ricinus communis | 276 | 330 |
| gma-miR164 | | XP_002310688 | Populus trichocarpa | 277 | 331 |
| gma-miR164 | | XP_002307195 | Populus trichocarpa | 278 | 332 |
| gma-miR164 | 720-740 | ACU24381 | Glycine max | 279 | 333 |
| gma-miR164 | | ACD39385 | Glycine max | 280 | 334 |
| gma-miR164 | | ACC66316 | Glycine max | 281 | 335 |
| gma-miR164 | | AAK84883 | Phaseolus vulgaris | 282 | 336 |
| gma-miR164 | | AEE99077 | Medicago | 283 | 337 |
| | | | truncatula | | |
| gma-miR164 | | XP_002529954 | Ricinus communis | 284 | 338 |
| gma-miR164 | | XP_002310688 | Populus trichocarpa | 285 | 339 |
| gma-miR164 | | XP_002307195 | Populus trichocarpa | 286 | 340 |
| osa- | 471-491 | ACU22861 | Glycine max | 287 | 341 |
| miR397b | | | | | |
| osa- | 702-722 | AAM54731 | Glycine max | 288 | 342 |
| miR397b | | | | | |
| osa- | | XP_002315131 | Populus trichocarpa | 289 | 343 |
| miR397b | | | | | |
| osa- | | CBI25418 | Vitis vinifera | 290 | 344 |
| miR397b | | | | | |
| osa- | | XP_002520796 | Ricinus communis | 291 | 345 |
| miR397b | | | | | |
| osa- | | ABK92474 | Populus trichocarpa | 292 | 346 |
| miR397b | | | | | |
| osa- | | XP_002273875 | Vitis vinifera | 293 | 347 |
| miR397b | | | | | |
| osa- | | XP_002312186 | Populus trichocarpa | 294 | 348 |
| miR397b | | | | | |
| osa- | | XP_002520797 | Ricinus communis | 295 | 349 |
| miR397b | | | | | |
| osa- | | XP_002881718 | Arabidopsis lyrata | 296 | 350 |
| miR397b | | | subsp. lyrata | | |
| osa- | | CAN75316 | Vitis vinifera | 297 | 351 |
| miR397b | | | | | |
| sof-miR408e | 44986 | ACU13981 | Glycine max | 298 | 352 |
| sof-miR408e | | ACJ84083 | Medicago | 299 | 353 |
| | | | truncatula | | |
| sof-miR408e | | ACU13882 | Glycine max | 300 | 354 |
| sof-miR408e | | CAA10134 | Cicer arietinum | 301 | 355 |
| sof-miR408e | | XP_002298184 | Populus trichocarpa | 302 | 356 |
| sof-miR408e | | ACU13343 | Glycine max | 303 | 357 |
| sof-miR408e | | XP_002520251 | Ricinus communis | 304 | 358 |
| sof-miR408e | 45383 | ACU13343 | Glycine max | 305 | 359 |
| sof-miR408e | | ACJ84194 | Medicago | 306 | 360 |
| | | | truncatula | | |
| smo- | 41-61 | ACU14055 | Glycine max | 307 | 361 |
| miR1093 | | | | | |
| smo- | | XP_002318822 | Populus trichocarpa | 308 | 362 |
| miR1093 | | | | | |
| smo- | | Q9FUL4 | Prunus avium | 309 | 363 |
| miR1093 | | | | | |
| smo- | | ACU13374 | Glycine max | 310 | 364 |
| miR1093 | | | | | |
| smo- | | 065743 | Cicer arietinum | 311 | 365 |
| miR1093 | | | | | |
| smo- | | XP_002283947 | Vitis vinifera | 312 | 366 |
| miR1093 | | | | | |
| smo- | | ACU14473 | Glycine max | 313 | 367 |
| miR1093 | | | | | |
| smo- | | ABE80118 | Medicago | 314 | 368 |
| miR1093 | | | truncatula | | |
| smo- | | AEC11017 | Camellia sinensis | 315 | 369 |
| miR1093 | | | | | |
| smo- | | ACJ86193 | Medicago | 316 | 370 |
| miR1093 | | | truncatula | | |

### Example 5

### Verification of Expression of Small RNA Molecules Associated with Abiotic Stress in Soybean Plants

Following identification of small RNA molecules potentially involved in improvement of soybean abiotic stress tolerance using bioinformatics tools, as described in Example 4 above, the actual mRNA levels in an experiment are determined using reverse transcription assay followed by quantitative Real-Time PCR (qRT-PCR) analysis. RNA levels are compared between different tissues, developmental stages, growing conditions and/or genetic backgrounds incorporated in each experiment. A correlation analysis between mRNA levels in different experimental conditions/genetic backgrounds is applied and used as evidence for the role of the gene in the plant.

### Methods

Root and leaf samples are freshly excised from soybean plants grown as described above on Murashige-Skoog (Duchefa). Experimental plants are grown either under optimal irrigation conditions to be used as a control group, or under stressful conditions of prolonged water deprivation to be used as a stress-induced group. Total RNA is extracted from the different tissues, using mirVana™ commercial kit (Ambion) following the protocol provided by the manufacturer. For measurement and verification of messenger RNA (mRNA) expression level of all genes, reverse transcription followed by quantitative real time PCR (qRT-PCR) is performed on total RNA extracted from each plant tissue (i.e., roots and leaves) from each experimental group as described above. To elaborate, reverse transcription is performed on 1 μg total RNA, using a miScript Reverse Transcriptase kit (Qiagen), following the protocol suggested by the manufacturer. Quantitative RT-PCR is performed on cDNA (0.1 ng/μl final concentration), using a miScript SYBR GREEN PCR (Qiagen) forward (based on the miR sequence itself) and reverse primers (supplied with the kit). All qRT-PCR reactions are performed in triplicates using an AB17500 real-time PCR machine, following the recommended protocol for the machine. To normalize the expression level of miRNAs associated with enhanced abiotic stress tolerance between the different tissues and growing conditions of the soybean plants, normalizer miRNAs are selected and used for comparison. Normalizer miRNAs, which are miRNAs with unchanged expression level between tissues and growing conditions, are custom selected for each experiment. The normalization procedure consists of second-degree polynomial fitting to a reference data (which is the median vector of all the data—excluding outliers) as described by Rosenfeld et al. (2008, Nat. Biotechnol., 26(4):462-469). A summary of primers for the differential small RNA molecules that will be used in the qRT-PCR validation and analysis is presented in Table 4 below.

TABLE 4
Primers for qRT-PCR Analysis of Small RNA Molecules Differentially
Expressed under Drought Stress.

| | miRNA | | |
|---|---|---|---|
| miRNA Name | SEQ ID NO | Primer SEQ ID NO | Primer Length |
| gma-miR164 | 1 | 371 | 21 |
| gma-miR168 | 2 | 372 | 21 |
| osa-miR397b | 3 | 373 | 24 |
| sof-miR408e | 4 | 374 | 21 |
| smo-miR1093 | 5 | 375 | 21 |

### Example 6

### Gene Cloning Strategies for miRNA Molecules and Creation of Binary Vectors for Plant Expression

The best validated miRNA sequences are cloned into pORE-E1 binary vectors for the generation of transgenic plants. The full-length precursor sequence comprising of the hairpin sequence of each selected miRNA, is synthesized by Genscript (USA). The resulting clone is digested with appropriate restriction enzymes and inserted into the Multi Cloning Site (MCS) of a similarly digested binary vector through ligation using T4 DNA ligase enzyme (Promega, Madison, Wis., USA).

### Example 7

### Generation of Transgenic Model Plants Expressing the Abiotic Stress Associated Small RNAs

Arabidopsis thaliana transformation is performed using the floral dip procedure following a slightly modified version of the published protocol (Clough and Bent, 1998, Plant J., 16(6):735-43; and Desfeux et al., 2000, Plant Physiol., 123(3):895-904). Briefly, T₀ plants are planted in small pots filled with soil. The pots are covered with aluminum foil and a plastic dome, kept at 4° C. for 3-4 days, then uncovered and incubated in a growth chamber at 24° C. under 16 hr light: 8 hr dark cycles. A week prior to transformation, all individual flowering stems are removed to allow for growth of multiple flowering stems instead. A single colony of Agrobacterium (GV3101) carrying the binary vectors (pORE-E1), harboring the NUE miRNA hairpin sequences with additional flanking sequences both upstream and downstream of it, is cultured in LB medium supplemented with kanamycin (50 mg/L) and gentamycin (25 mg/L). Three days prior to transformation, each culture is incubated at 28° C. for 48 hrs, shaking at 180 rpm. The starter culture is split the day before transformation into two cultures, which are allowed to grow further at 28° C. for 24 hours at 180 rpm. Pellets containing the agrobacterium cells are obtained by centrifugation of the cultures at 5000 rpm for 15 minutes. The pellets are re-suspended in an infiltration medium (10 mM MgCl₂, 5% sucrose, 0.044 μM BAP (Sigma) and 0.03% Tween 20) prepared with double-distilled water.

Transformation of T0 plants is performed by inverting each plant into the agrobacterium suspension, keeping the flowering stem submerged for 5 minutes. Following inoculation, each plant is blotted dry for 5 minutes on both sides, and placed sideways on a fresh covered tray for 24 hours at 22° C. Transformed (transgenic) plants are then uncovered and transferred to a greenhouse for recovery and maturation. The transgenic T0 plants are grown in the greenhouse for 3-5 weeks until the seeds are ready, which are then harvested from plants and kept at room temperature until sowing.

### Example 8

### Selection of Transgenic Arabidopsis Plants Expressing the Abiotic Stress Genes According to Expression Level

Arabidopsis seeds are sown and Basta (Bayer) is sprayed for the first time on 1-2 weeks old seedlings, at least twice every few days. Only resistant plants, which are heterozygous for the transgene, survive. PCR on the genomic gene sequence is performed on the surviving seedlings using primers pORE-F2 (fwd, 5′-TTTAGCGATGAACTTCACTC-3′, SEQ ID NO:377) and a custom-designed reverse primer based on each small RNA sequence.

### Example 9

### Evaluating Changes in Root Architecture in Transgenic Plants

Many key traits in modern agriculture can be explained by changes in the root architecture of the plant. Root size and depth have been shown to logically correlate with drought tolerance and fertilizer use efficiency, since deeper and more branched root systems provide better coverage of the soil and can access water stored in deeper soil layers.

To test whether the transgenic plants produce a modified root structure, plants can be grown in agar plates placed vertically. A digital picture of the plates is taken every few days and the maximal length and total area covered by the plant roots are assessed. From every construct created, several independent transformation events are checked in replicates. To assess significant differences between root features, a statistical test, such as Student's t-test, is employed in order to identify enhanced root features and to provide a statistical value to the findings.

### Example 10

### Testing for Increased Abiotic Stress Tolerance

To analyze whether the transgenic Arabidopsis plants are more tolerant to abiotic stresses, plants are grown under optimal versus stress conditions, e.g., drought (no irrigation for one week). Plants are allowed to grow until seed production followed by an analysis of their overall size, time to flowering, yield, and protein content of shoot and/or grain. The parameters checked can be the overall size of the plant, wet and dry weight, the weight of the seeds yielded, the average seed size, and the number of seeds produced per plant. Transformed plants not exhibiting substantial physiological and/or morphological effects, or exhibiting higher measured parameter levels compared to wild-type plants, are identified as abiotic stress tolerant plants.

### Example 11

### Method for Generating Transgenic Soybean Plants with Enhanced or Reduced small RNA Regulation of Target Genes

Target prediction enables two contrasting strategies: an enhancement (positive) or a reduction (negative) of small RNA regulation. Both these strategies have been used in plants and have resulted in significant phenotype alterations. For complete in-vivo assessment of the phenotypic effects of the differential small RNAs of this disclosure, the inventors plan to implement both overexpression and downregulation methods on the small RNA molecules found to associate with abiotic stress tolerance as listed in Table 1. In the case of small RNAs that were upregulated under abiotic stress conditions, an enhancement in abiotic stress tolerance can be achieved by maintaining their directionality, e.g., overexpressing them. Conversely, in the case of small RNAs that were downregulated under abiotic stress conditions, enhancement in tolerance can be achieved by reduction of their regulation. Reduction of small RNA regulation of target genes can be accomplished in one of the following approaches:

(a) Expressing a miRNA-Resistant Target

In this method, silent mutations are introduced in the miRNA binding site of the target gene so that the DNA and resulting RNA sequences are changed to prevent miRNA binding, but the amino acid sequence of the protein is unchanged.

For design of miRNA-resistant target sequences for the small RNA molecules of the disclosure, optimization of the nucleic acid sequence in accordance with the preferred codon usage for a particular plant species is required. Tables such as those provided on-line at the Codon Usage Database through the NCBI (National Center for Biotechnology Information) webpage (available on the internet at www.ncbi.nlm.nih.gov/Taxonomy/Utils/wprintgc.cgi) are used. The Genbank database contains codon usage tables for a number of different species, with its Table 11 (The Bacterial, Archaeal and Plant Plastid Code) being the most relevant for plant species of this disclosure.

(b) Expressing a Target-Mimic Sequence

Plant miRNAs usually lead to cleavage of their targeted gene, with this cleavage typically occurring between bases 10 and 11 of the miRNA. This position is therefore especially sensitive to mismatches between the miRNA and the target. It is found that expressing a DNA sequence that could potentially be targeted by a miRNA, but contains three extra nucleotides (ATC), and thus creates a bulge in a key position (between the two nucleotides that are predicted to hybridize with bases 10-11 of the miRNA), can inhibit the regulation of that miRNA on its native targets (Franco-Zorilla et al., 2007, Nat. Genet., 39(8):1033-1037).

This type of sequence is referred to as a “target-mimic.” Inhibition of the miRNA regulation is presumed to occur through physically capturing the miRNA by the target-mimic sequence and titering-out the miRNA, thereby reducing its abundance. This method was used to reduce the amount and, consequentially, the regulation of miRNA 399 in Arabidopsis.

(c) Soybean Transformation

This example illustrates plant transformation useful in producing a transgenic soybean plant cell, and a transgenic plant having an enhanced trait, e.g., enhanced water use efficiency, enhanced cold tolerance, increased yield, enhanced nitrogen use efficiency, enhanced seed protein, and enhanced seed oil.

For Agrobacterium-mediated transformation, soybean seeds are imbibed overnight and the meristem explants excised. The explants are placed in a wounding vessel. Soybean explants and induced Agrobacterium cells from a strain containing plasmid DNA with the gene of interest cassette and a plant selectable marker cassette are mixed no later than 14 hours from the time of initiation of seed imbibition, and wounded using sonication. Following wounding, explants are placed in co-culture for 2-5 days at which point they are transferred to selection media for 6-8 weeks to allow selection and growth of transgenic shoots. Resistant shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

TABLE 5
miRNA-Resistant Target Examples for Selected miRNAs which were
Downregulated under Drought Stress.

| | | | SEQ ID | | SEQ ID NO of | |
|---|---|---|---|---|---|---|
| | | | NO of | | Mutated | |
| | NCBI | | Original | SEQ ID | miRNA- | miRNA |
| miRNA | Accession | Protein | Nucleotide | NO of ORF | Resistant | Binding |
| Name | of a Target | SEQ ID NO | Sequence | Sequence | Sequence | Site |
| osa-miR397b | ACU22861 | 376 | 380 | 383 | 386 | 469-489 |
| osa-miR397b | ACU22861 | 376 | 380 | 383 | 387 | 469-489 |
| osa-miR397b | ACU22861 | 376 | 380 | 383 | 388 | 469-489 |
| osa-miR397b | ACU22861 | 376 | 380 | 383 | 389 | 469-489 |
| osa-miR397b | ACU22861 | 376 | 380 | 383 | 390 | 469-489 |
| osa-miR397b | ACU22861 | 376 | 380 | 383 | 391 | 469-489 |
| osa-miR397b | ACU22861 | 376 | 380 | 383 | 392 | 469-489 |
| osa-miR397b | ACU22861 | 376 | 380 | 383 | 393 | 469-489 |
| osa-miR397b | ACU22861 | 376 | 380 | 383 | 394 | 469-489 |
| osa-miR397b | ACU22861 | 376 | 380 | 383 | 395 | 469-489 |
| osa-miR397b | AAM54731 | 378 | 381 | 384 | 396 | 701-721 |
| osa-miR397b | AAM54731 | 378 | 381 | 384 | 397 | 701-721 |
| osa-miR397b | AAM54731 | 378 | 381 | 384 | 398 | 701-721 |
| osa-miR397b | AAM54731 | 378 | 381 | 384 | 399 | 701-721 |
| osa-miR397b | AAM54731 | 378 | 381 | 384 | 400 | 701-721 |
| osa-miR397b | AAM54731 | 378 | 381 | 384 | 401 | 701-721 |
| osa-miR397b | AAM54731 | 378 | 381 | 384 | 402 | 701-721 |
| osa-miR397b | AAM54731 | 378 | 381 | 384 | 403 | 701-721 |
| osa-miR397b | AAM54731 | 378 | 381 | 384 | 404 | 701-721 |
| osa-miR397b | AAM54731 | 378 | 381 | 384 | 405 | 701-721 |
| sof-miR408e | ACU13343 | 379 | 382 | 385 | 406 | 52-72 |
| sof-miR408e | ACU13343 | 379 | 382 | 385 | 407 | 52-72 |
| sof-miR408e | ACU13343 | 379 | 382 | 385 | 408 | 52-72 |
| sof-miR408e | ACU13343 | 379 | 382 | 385 | 409 | 52-72 |
| sof-miR408e | ACU13343 | 379 | 382 | 385 | 410 | 52-72 |

TABLE 6
Target Mimic Examples for Selected miRNAs of the Disclosure which
were Downregulated under Drought Stress.

| | | SEQ ID NO | SEQ ID NO | SEQ ID NO |
|---|---|---|---|---|
| | | of Reverse | of Bulge- | of |
| | | Complement | containing | Full-length |
| | miRNA | Sequence | Target | Target |
| | SEQ ID | of Bulge | Binding | Mimic |
| mirRNA Name | NO | miR NA | Sequence | Sequence |
| osa-miR397b | 3 | 411 | 414 | 417 |
| sof-miR408e | 4 | 412 | 415 | 418 |
| smo-miR1093 | 5 | 413 | 416 | 419 |

TABLE 7
Abbreviations of Plant Species

| Abbreviation | Organism Full Name | Common Name |
|---|---|---|
| ahy | Arachis hypogaea | Peanut |
| aly | Arabidopsis lyrata | Arabidopsis lyrata |
| aqc | Aquilegia coerulea | Rocky Mountain Columbine |
| ath | Arabidopsis thaliana | Arabidopsis thaliana |
| bdi | Brachypodium distachyon | Grass |
| bna | Brassica napus | Brassica napus canola (“liftit”) |
| bra | Brassica rapa | Brassica rapa yellow mustard |
| ccl | Citrus clementine | Clementine |
| crt | Citrus reticulata | Mandarin |
| csi | Citrus sinensis | Orange |
| ctr | Citrus trifoliata | Trifoliate orange |
| far | Festuca arundinacea | Tall fescue |
| ghr | Gossypium hirsutum | Gossypium hirsutum cotton |
| gma | Glycine max | Glycine max |
| hvu | Hordeum vulgare | Barley |
| mtr | Medicago truncatula | Medicago truncatula - Barrel Clover (“tiltan”) |
| osa | Oryza sativa | Oryza sativa |
| pab | Picea abies | European spruce |
| ppt | Physcomitrella patens | Physcomitrella patens (moss) |
| pta | Pinus taeda | Pinus taeda - Loblolly Pine |
| ptc | Populus trichocarpa | Populus trichocarpa - black cotton wood |
| rco | Ricinus communis | Castor Bean |
| sbi | Sorghum bicolor | Sorghum bicolor Dura |
| sly | Solanum lycopersicum | Solanum lycopersicum tomato |
| smo | Selaginella moellendorffii | Selaginella moellendorffii |
| sof | Saccharum officinarum | Sugarcane |
| ssp | Saccharum spp. | Sugarcane |
| tae | Triticum aestivum | Triticum aestivum |
| tcc | Theobroma cacao | cacao tree |
| vvi | Vitis vinifera | Vitis vinifera Grapes |
| zma | Zea mays | corn |

1. A method of improving abiotic stress tolerance in a soybean plant comprising transgenically expressing in said soybean plant a recombinant DNA construct comprising a heterologous promoter operably linked to at least one DNA selected from the group consisting of:
a. a DNA encoding at least one miRNA precursor that yields a mature miRNA selected from the group consisting of a mature miR164 and a mature miR168;
b. a DNA encoding a miR397 target mimic, a miR408 target mimic, or a miR1093 target mimic; and
c. a DNA encoding a miR397-, miR408-, or miR 1093-resistant target gene, wherein said miR397-, miR408-, or miR 1093-resistant target gene comprises an introduced silent mutation in a nucleotide sequence that is otherwise substantially identical to the nucleotide sequence of an endogenous gene that is natively regulated by miR397, miR408, or miR1093, and wherein said silent mutation prevents binding by a mature miR397, miR408, or miR1093 to a transcript of said miR397-, miR408-, or miR 1093-resistant target gene.

2. The method of claim 1, wherein said at least one DNA is further selected from the group consisting of:
a. a DNA encoding at least one miRNA precursor comprising a nucleotide sequence selected from the group consisting of SEQ ID NOs: 1, 2, and 11-94;
b. a DNA encoding a miR397 target mimic, a miR408 target mimic, or a miR1093 target mimic comprising a nucleotide sequence as set forth in SEQ ID NOs: 414, 415, or 416;
c. a DNA encoding a miR397 target mimic, a miR408 target mimic, or a miR1093 target mimic comprising a nucleotide sequence as set forth in SEQ ID NOs: 417, 418, or 419; and
d. a DNA encoding a miR397-, miR408-, or miR 1093-resistant target gene selected from the group consisting of SEQ ID NOs: 386-410.

3-5. (canceled)

6. The method of claim 1, wherein the abiotic stress is drought, osmotic stress, heat stress, or cold stress.

7. The method of claim 1, wherein the heterologous promoter is a constitutive promoter or an inducible promoter.

8. The method of claim 1, wherein the heterologous promoter is a CaMV 35S promoter or an abiotic stress inducible promoter.

9-10. (canceled)

11. The method of claim 1, wherein said soybean plant further comprises a DNA sequence encoding a protein that provides tolerance to an herbicide.

12. The method of claim 1, wherein the herbicide is selected from the group consisting of glyphosate, 2,4-dichloropropionic acid, bromoxynil, sulfonylurea, imidazolinone, triazolopyrimidine, pyrimidyloxybenzoates, phthalide, bialaphos, phosphinothricin, glufosinate, atrazine, dicamba, cyclohexanedione (sethoxydim), and aryloxyphenoxypropionate (haloxyfop).

13. A method of providing a plant with increased root branching or root depth comprising transgenically expressing in said plant a recombinant DNA construct comprising a heterologous promoter operably linked to at least one DNA selected from the group consisting of:
a. a DNA encoding at least one miRNA precursor that yields a mature miRNA selected from the group consisting of a mature miR 164 and a mature miR 168;
b. a DNA encoding a miR397 target mimic, a miR408 target mimic, or a miR1093 target mimic; and
c. a DNA encoding a miR397-, miR408-, or miR 1093-resistant target gene, wherein said miR397-, miR408-, or miR 1093-resistant target gene comprises an introduced silent mutation in a nucleotide sequence that is otherwise substantially identical to the nucleotide sequence of an endogenous gene that is natively regulated by miR397, miR408, or miR1093, and wherein said silent mutation prevents binding by a mature miR397, miR408, or miR1093 to a transcript of said miR397-, miR408-, or miR 1093-resistant target gene.

14. The method of claim 13, wherein said at least one DNA is further selected from the group consisting of:
a. a DNA encoding at least one miRNA precursor comprising a nucleotide sequence selected from the group consisting of SEQ ID NOs: 1, 2, and 11-94;
b. a DNA encoding a miR397 target mimic, a miR408 target mimic, or a miR1093 target mimic comprising a nucleotide sequence as set forth in SEQ ID NOs: 414, 415, or 416;
c. a DNA encoding a miR397 target mimic, a miR408 target mimic, or a miR1093 target mimic comprising a nucleotide sequence as set forth in SEQ ID NOs: 417, 418, or 419; and
d. a DNA encoding a miR397-, miR408-, or miR 1093-resistant target gene selected from the group consisting of SEQ ID NOs: 386-410.

15-17. (canceled)

18. The method of claim 13, wherein the heterologous promoter is a constitutive promoter or an inducible promoter.

19. The method of claim 13, wherein the heterologous promoter is a CaMV 35S promoter or an abiotic stress inducible promoter.

20-21. (canceled)

22. A method of producing a transgenic soybean plant, said method comprising:
transforming a soybean plant cell with a transgene selected from the group consisting of:
a. a transgene comprising a heterologous promoter operably linked to at least one DNA encoding a mature miRNA, comprising a sequence having at least 90% sequence identity to a sequence selected from the group consisting of SEQ ID NOs: 1, 2, and 11-94; and
b. a transgene comprising a heterologous promoter operably linked to at least one DNA having at least 90% sequence identity to a sequence selected from the group consisting of SEQ ID NOs: 417-419 and 386-410, and
producing a transgenic soybean plant from said transformed cell,
wherein said transgenic soybean plant has improved drought tolerance compared to a control soybean plant lacking said transgene.

23. (canceled)

24. The method of claim 22, wherein said transgene is stably integrated into the genome of said transgenic soybean plant.

25. The method of claim 22, wherein said improved drought tolerance is measured by an increase of at least 1% in water use efficiency (WUE) when said transgenic and control soybean plants are grown under similar drought conditions, and said WUE is measured by the amount of biomass accumulated per unit of water used.

26. (canceled)

27. A transgenic soybean plant produced by the method of claim 22, or a part thereof.

28. The transgenic soybean plant or a part thereof of claim 27, comprising a transgene selected from the group consisting of:
a. a transgene that encodes a mature miRNA comprising a sequence having at least 90% sequence identity to a sequence selected from the group consisting of SEQ ID NOs: 1, 2, and 11-94;
b. a transgene that encodes a miR397 target mimic, a miR408 target mimic, or a miR1093 target mimic;
c. a miR397 target mimic, a miR408 target mimic, or a miR1093 target mimic comprising a sequence having at least 90% sequence identity to a sequence selected from the group consisting of SEQ ID NOs: 417-419; and
d. a transgene that encodes a miR397-, miR408-, or miR 1093-resistant target gene, wherein said miR397-, miR408-, or miR 1093-resistant target gene comprises a sequence having at least 90% sequence identity to a sequence selected from the group consisting of SEQ ID NOs: 386-410,
wherein said transgenic soybean plant has improved drought tolerance compared to a non-transgenic control soybean plant.

29-30. (canceled)

31. The transgenic soybean plant, or part thereof, of claim 27, wherein said transgene further comprises a DNA sequence encoding a protein that provides tolerance to an herbicide.

32. The transgenic soybean plant, or part thereof, of claim 31, wherein said herbicide is selected from the group consisting of glyphosate, 2,4-dichloropropionic acid, bromoxynil, sulfonylurea, imidazolinone, triazolopyrimidine, pyrimidyloxybenzoates, phthalide, bialaphos, phosphinothricin, glufosinate, atrazine, dicamba, cyclohexanedione (sethoxydim), and aryloxyphenoxypropionate (haloxyfop).

33. The transgenic soybean plant, or part thereof, of claim 27, wherein said part is selected from the group consisting of a leaf, a stem, a root, a seed, a flower, pollen, an anther, an ovule, a pedicel, a fruit, a meristem, a cotyledon, a hypocotyl, a pod, an embryo, endosperm, an exsoybean plant, a callus, a tissue culture, a shoot, a cell, and a protoplast.

34. A soybean product made from the transgenic soybean plant, or a part thereof, of claim 27.


SEQUENCE LISTING (419 sequences)

SEQ ID NO: 1 - DNA, 21 bp, Glycine max
tggagaagcagggcacgtgca

SEQ ID NO: 2 - DNA, 21 bp, Glycine max
tcgcttggtgcaggtcgggaa

SEQ ID NO: 3 - DNA, 21 bp, Oryza sativa
ttattgagtgcagcgttgatg

SEQ ID NO: 4 - DNA, 21 bp, Saccharum officinarum
ctgcactgactcttccctggc

SEQ ID NO: 5 - DNA, 21 bp, Selaginella moellendorffii
tggaggtgtcgttgccaagga

SEQ ID NO: 6 - RNA, 95 nt, Glycine max
agcuccuuguuggagaagcagggcacgugcaagucucuuggaucucaaaugccacugaacccuuugcacgugcuccccuucuccaacacggguuu

SEQ ID NO: 7 - RNA, 125 nt, Glycine max
cacugugcggucucuaauucgcuuggugcaggucgggaaccgguuuucgcgcggaauggaggagcggucgccggcgccgaauuggaucccgccuugcaucaacugaaucggaggccgcggugaac

SEQ ID NO: 8 - RNA, 118 nt, Oryza sativa
agggaaggcauuauugagugcagcguugaugaaccugccggccggcuaaauuaauuagcaagaaagucugaaacuggcucaaagguucaccagcacugcacccaaucacgccuuugcu

SEQ ID NO: 9 - RNA, 283 nt, Saccharum officinarum
agaagauggguaugguuggagacagggaugaggcagagcaugggaugaggccaucaacaaaauuuccaauuucuguccuccgcuaggccgcuacugcauuuauguuugcuugcucacaaaacggagggauuugugagaguuaucaggcagaaagaacaaagaaggugccucccuggugaaguggugauggccugaccugagacggaugagagcucagcugguguccuguuguugcuuacuucccugcacugacucuucccuggcuccccaccguugcccuugc

SEQ ID NO: 10 - RNA, 90 nt, Selaginella moellendorffii
uuucaccacaccauuggugauggaggugucguugccaaggaaacuuuccuuagcuuuccuuggcgccgacaccucugccaucaccaaugg

SEQ ID NO: 11 - DNA, 21 bp, Arabidopsis lyrata
tggagaagcagggcacgtgca

SEQ ID NO: 12 - DNA, 21 bp, Arabidopsis lyrata
tggagaagcagggcacgtgca

SEQ ID NO: 13 - DNA, 21 bp, Arabidopsis lyrata
tggagaagcagggcacgtgcg

SEQ ID NO: 14 - DNA, 21 bp, Arabidopsis thaliana
tggagaagcagggcacgtgca

SEQ ID NO: 15 - DNA, 21 bp, Arabidopsis thaliana
tggagaagcagggcacgtgca

SEQ ID NO: 16 - DNA, 21 bp, Arabidopsis thaliana
tggagaagcagggcacgtgcg

SEQ ID NO: 17 - DNA, 21 bp, Brachypodium distachyon
tggagaagcagggcacgtgca

SEQ ID NO: 18 - DNA, 21 bp, Brachypodium distachyon
tggagaagcagggcacgtgca

SEQ ID NO: 19 - DNA, 21 bp, Brachypodium distachyon
tggagaagcagggcacgtgct

SEQ ID NO: 20 - DNA, 21 bp, Brachypodium distachyon
tggagaagcagggcacgtgca

SEQ ID NO: 21 - DNA, 21 bp, Brachypodium distachyon
tggagaagcagggcacgtgca

SEQ ID NO: 22 - DNA, 21 bp, Brachypodium distachyon
tggagaagaagggcacatgca

SEQ ID NO: 23 - DNA, 21 bp, Brassica napus
tggagaagcagggcacgtgca

SEQ ID NO: 24 - DNA, 21 bp, Brassica rapa
tggagaagcagggcacgtgca

SEQ ID NO: 25 - DNA, 21 bp, Citrus sinensis
tggagaagcagggcacgtgca

SEQ ID NO: 26 - DNA, 21 bp, Citrus trifoliata
tggagaagcagggcacgtgca

SEQ ID NO: 27 - DNA, 21 bp, Festuca arundinacea
tggagaagcagggcacttgct

SEQ ID NO: 28 - DNA, 21 bp, Festuca arundinacea
tggagaagcagggcacttgct

SEQ ID NO: 29 - DNA, 21 bp, Gossypium hirsutum
tggagaagcagggcacgtgca

SEQ ID NO: 30 - DNA, 21 bp, Medicago truncatula
tggagaagcagggcacgtgca

SEQ ID NO: 31 - DNA, 21 bp, Medicago truncatula
tggagaagcagggcacgtgca

SEQ ID NO: 32 - DNA, 21 bp, Medicago truncatula
tggagaagcagggcacgtgca

SEQ ID NO: 33 - DNA, 21 bp, Medicago truncatula
tggagaagcagggcacatgct

SEQ ID NO: 34 - DNA, 21 bp, Oryza sativa
tggagaagcagggcacgtgca

SEQ ID NO: 35 - DNA, 21 bp, Oryza sativa
tggagaagcagggcacgtgca

SEQ ID NO: 36 - DNA, 21 bp, Oryza sativa
tggagaagcagggtacgtgca

SEQ ID NO: 37 - DNA, 21 bp, Oryza sativa
tggagaagcagggcacgtgct

SEQ ID NO: 38 - DNA, 21 bp, Oryza sativa
tggagaagcagggcacgtgag

SEQ ID NO: 39 - DNA, 21 bp, Oryza sativa
tggagaagcagggcacgtgca

SEQ ID NO: 40 - DNA, 21 bp, Populus trichocarpa
tggagaagcagggcacgtgca

SEQ ID NO: 41 - DNA, 21 bp, Populus trichocarpa
tggagaagcagggcacgtgca

SEQ ID NO: 42 - DNA, 21 bp, Populus trichocarpa
tggagaagcagggcacgtgca

SEQ ID NO: 43 - DNA, 21 bp, Populus trichocarpa
tggagaagcagggcacgtgca

SEQ ID NO: 44 - DNA, 21 bp, Populus trichocarpa
tggagaagcagggcacgtgca

SEQ ID NO: 45 - DNA, 21 bp, Populus trichocarpa
tggagaagcagggcacatgct

SEQ ID NO: 46 - DNA, 21 bp, Ricinus communis
tggagaagcagggcacgtgca

SEQ ID NO: 47 - DNA, 21 bp, Ricinus communis
tggagaagcagggcacgtgca

SEQ ID NO: 48 - DNA, 21 bp, Ricinus communis
tggagaagcagggcacgtgca

SEQ ID NO: 49 - DNA, 21 bp, Ricinus communis
tggagaagcagggcacatgct

SEQ ID NO: 50 - DNA, 21 bp, Sorghum bicolor
tggagaagcagggcacgtgca

SEQ ID NO: 51 - DNA, 21 bp, Sorghum bicolor
tggagaagcagggcacgtgct

SEQ ID NO: 52 - DNA, 21 bp, Sorghum bicolor
tggagaagcaggacacgtgag

SEQ ID NO: 53 - DNA, 21 bp, Sorghum bicolor
tggagaagcagggcacgtgca

SEQ ID NO: 54 - DNA, 21 bp, Sorghum bicolor
tggagaagcagggcacgtgca

SEQ ID NO: 55 - DNA, 21 bp, Triticum aestivum
tggagaagcagggcacgtgca

SEQ ID NO: 56 - DNA, 21 bp, Theobroma cacao
tggagaagcagggcacgtgca

SEQ ID NO: 57 - DNA, 21 bp, Theobroma cacao
tggagaagcagggcacgtgca

SEQ ID NO: 58 - DNA, 21 bp, Theobroma cacao
tggagaagcagggcacatgct

SEQ ID NO: 59 - DNA, 21 bp, Vitis vinifera
tggagaagcagggcacgtgca

SEQ ID NO: 60 - DNA, 21 bp, Vitis vinifera
tggagaagcagggcacatgct

SEQ ID NO: 61 - DNA, 21 bp, Vitis vinifera
tggagaagcagggcacgtgca

SEQ ID NO: 62 - DNA, 21 bp, Vitis vinifera
tggagaagcagggcacgtgca

SEQ ID NO: 63 - DNA, 21 bp, Zea mays
tggagaagcagggcacgtgca

SEQ ID NO: 64 - DNA, 21 bp, Zea mays
tggagaagcagggcacgtgca

SEQ ID NO: 65 - DNA, 21 bp, Zea mays
tggagaagcagggcacgtgca

SEQ ID NO: 66 - DNA, 21 bp, Zea mays
tggagaagcagggcacgtgca

SEQ ID NO: 67 - DNA, 21 bp, Zea mays
tggagaagcaggacacgtgag

SEQ ID NO: 68 - DNA, 21 bp, Zea mays
tggagaagcagggcacgtgct

SEQ ID NO: 69 - DNA, 21 bp, Zea mays
tggagaagcagggcacgtgca

SEQ ID NO: 70 - DNA, 21 bp, Zea mays
tggagaagcagggcacgtgtg

SEQ ID NO: 71 - DNA, 21 bp, Arabidopsis lyrata
tcgcttggtgcaggtcgggaa

SEQ ID NO: 72 - DNA, 21 bp, Arabidopsis lyrata
tcgcttggtgcaggtcgggaa

SEQ ID NO: 73 - DNA, 21 bp, Aquilegia coerulea
tggcttagtgcagctcgggga

SEQ ID NO: 74 - DNA, 21 bp, Arabidopsis thaliana
tcgcttggtgcaggtcgggaa

SEQ ID NO: 75 - DNA, 21 bp, Arabidopsis thaliana
tcgcttggtgcaggtcgggaa

SEQ ID NO: 76 - DNA, 21 bp, Brachypodium distachyon
tcgcttggtgcagatcgggac

SEQ ID NO: 77 - DNA, 21 bp, Brassica napus
tcgcttggtgcaggtcgggaa

SEQ ID NO: 78 - DNA, 21 bp, Citrus clementine
tcgcttggtgcaggtcgggaa

SEQ ID NO: 79 - DNA, 21 bp, Citrus trifoliata
tcgcttggtgcaggtcgggaa

SEQ ID NO: 80 - DNA, 21 bp, Hordeum vulgare
tcgcttggtgcagatcgggac

SEQ ID NO: 81 - DNA, 21 bp, Medicago truncatula
ttgcttggtgctggtcgggaa

SEQ ID NO: 82 - DNA, 21 bp, Oryza sativa
tcgcttggtgcagatcgggac

SEQ ID NO: 83 - DNA, 21 bp, Oryza sativa
aggcttggtgcagctcgggaa

SEQ ID NO: 84 - DNA, 21 bp, Populus trichocarpa
tcgcttggtgcaggtcgggaa

SEQ ID NO: 85 - DNA, 21 bp, Populus trichocarpa
tcgcttggtgcaggtcgggaa

SEQ ID NO: 86 - DNA, 21 bp, Ricinus communis
tcgcttggtgcaggtcgggaa

SEQ ID NO: 87 - DNA, 21 bp, Sorghum bicolor
tcgcttggtgcagatcgggac

SEQ ID NO: 88 - DNA, 21 bp, Saccharum officinarum
tcgcttggtgcagatcgggac

SEQ ID NO: 89 - DNA, 20 bp, Saccharum officinarum
tcgcttgggcagatcgggac

SEQ ID NO: 90 - DNA, 21 bp, Saccharum spp.
tcgcttggtgcagatcgggac

SEQ ID NO: 91 - DNA, 21 bp, Theobroma cacao
tcgcttggtgcaggtcgggaa

SEQ ID NO: 92 - DNA, 21 bp, Vitis vinifera
tcgcttggtgcaggtcgggaa

SEQ ID NO: 93 - DNA, 21 bp, Zea mays
tcgcttggtgcagatcgggac

SEQ ID NO: 94 - DNA, 21 bp, Zea mays
tcgcttggtgcagatcgggac

SEQ ID NO: 95 - DNA, 21 bp, Arabidopsis lyrata
tcattgagtgcagcgttgatg

SEQ ID NO: 96 - DNA, 21 bp, Arabidopsis thaliana
tcattgagtgcagcgttgatg

SEQ ID NO: 97 - DNA, 21 bp, Brachypodium distachyon
tcattgagtgcagcgttgatg

SEQ ID NO: 98 - DNA, 21 bp, Brachypodium distachyon
attgagtgcagcgttgatgaa

SEQ ID NO: 99 - DNA, 22 bp, Brassica napus
tcattgagtgcagcgttgatgt

SEQ ID NO: 100 - DNA, 22 bp, Brassica napus
tcattgagtgcagcgttgatgt

SEQ ID NO: 101 - DNA, 21 bp, Citrus sinensis
tcattgagtgcagcgttgatg

SEQ ID NO: 102 - DNA, 21 bp, Hordeum vulgare
ccgttgagtgcagcgttgatg

SEQ ID NO: 103 - DNA, 21 bp, Oryza sativa
tcattgagtgcagcgttgatg

SEQ ID NO: 104 - DNA, 21 bp, Picea abies
tcattgagtgcagcgttgacg

SEQ ID NO: 105 - DNA, 21 bp, Populus trichocarpa
tcattgagtgcagcgttgatg

SEQ ID NO: 106 - DNA, 21 bp, Populus trichocarpa
ccattgagtgcagcgttgatg

SEQ ID NO: 107 - DNA, 21 bp, Populus trichocarpa
tcattgagtggagctttgatg

SEQ ID NO: 108 - DNA, 21 bp, Ricinus communis
tcattgagtgcagcgttgatg

SEQ ID NO: 109 - DNA, 21 bp, Sorghum bicolor
tcattgagtgcagcgttgatg

SEQ ID NO: 110 - DNA, 20 bp, Solanum lycopersicum
attgagtgcagcgttgatga

SEQ ID NO: 111 - DNA, 21 bp, Theobroma cacao
tcattgagtgcagcgttgatg

SEQ ID NO: 112 - DNA, 21 bp, Vitis vinifera
tcattgagtgcagcgttgatg

SEQ ID NO: 113 - DNA, 21 bp, Zea mays
tcattgagcgcagcgttgatg

SEQ ID NO: 114 - DNA, 21 bp, Zea mays
tcattgagcgcagcgttgatg

SEQ ID NO: 115 - DNA, 21 bp, Arachis hypogaea
atgcactgcctcttccctggc

SEQ ID NO: 116 - DNA, 21 bp, Arabidopsis lyrata
atgcactgcctcttccctggc

SEQ ID NO: 117 - DNA, 21 bp, Arabidopsis thaliana
atgcactgcctcttccctggc

SEQ ID NO: 118 - DNA, 21 bp, Citrus sinensis
atgcactgcctcttccctggc

SEQ ID NO: 119 - DNA, 21 bp, Oryza sativa
ctgcactgcctcttccctggc

SEQ ID NO: 120 - DNA, 22 bp, Physcomitrella patens
ctgcactgcatcttccctgtgc

SEQ ID NO: 121 - DNA, 21 bp, Physcomitrella patens
tgcactgcctcttccctggct

SEQ ID NO: 122 - DNA, 21 bp, Pinus taeda
atgcactgcctcttccctggc

SEQ ID NO: 123 - DNA, 21 bp, Populus trichocarpa
atgcactgcctcttccctggc

SEQ ID NO: 124 - DNA, 21 bp, Ricinus communis
ctgcactgcctcttccctggc

SEQ ID NO: 125 - DNA, 21 bp, Sorghum bicolor
ctgcactgcctcttccctggc

SEQ ID NO: 126 - DNA, 22 bp, Selaginella moellendorffii
tgcactgcctcttccctggctg

SEQ ID NO: 127 - DNA, 21 bp, Saccharum officinarum
ctgcactgcctcttccctggc

SEQ ID NO: 128 - DNA, 21 bp, Saccharum officinarum
ctgcactgcctcttccctggc

SEQ ID NO: 129 - DNA, 21 bp, Saccharum officinarum
ctgcactgcctcttccctggc

SEQ ID NO: 130 - DNA, 21 bp, Saccharum officinarum
ctgcactgcctcttccctggc

SEQ ID NO: 131 - DNA, 21 bp, Saccharum spp.
ctgcactgcctcttccctggc

SEQ ID NO: 132 - DNA, 21 bp, Saccharum spp.
ctgcactgcctcttccctggc

SEQ ID NO: 133 - DNA, 21 bp, Triticum aestivum
ctgcactgcctcttccctggc

SEQ ID NO: 134 - DNA, 21 bp, Vitis vinifera
atgcactgcctcttccctggc

SEQ ID NO: 135 - DNA, 21 bp, Zea mays
ctgcactgcctcttccctggc

SEQ ID NO: 136 - DNA, 21 bp, Zea mays
ctgcactgcctcttccctggc

SEQ ID NO: 137 - DNA, 79 bp, Arabidopsis lyrata
gttggagaagcagggcacgtgcaaaccaacaaacacgaaatccgtctcatgtgttttgcacgtactccccttctccaac

SEQ ID NO: 138 - DNA, 161 bp, Arabidopsis lyrata
gatggagaagcagggcacgtgcattactagctcatctcgtatgcatatatatatacatcctcaccacaaatgcgtgtatatgtgcggagatatagatatatatgtgtgtggagtgtgatgatataaatgagttagttcttcatgtgcccatcttcaccatc

SEQ ID NO: 139 - DNA, 115 bp, Arabidopsis lyrata
gggtgagtaacacttgatggagaagcagggcacgtgcgaacacaaatgagatcgatcggtacatgatgatcatattttcgcacgtgttctactcctccaacacgtgtctctcccc

SEQ ID NO: 140 - DNA, 113 bp, Arabidopsis thaliana
gggtgagaatctccatgttggagaagcagggcacgtgcaaaccaacaaacacgaaatccgtctcatttgcttatttgcacgtacttaacttctccaacatgagctcttcaccc

SEQ ID NO: 141 - DNA, 153 bp, Arabidopsis thaliana
gatggagaagcagggcacgtgcattactagctcatatatacactctcaccacaaatgcgtgtatatatgcggaattttgtgatatagatgtgtgtgtgtgttgagtgtgatgatatggatgagttagttcttcatgtgcccatcttcaccatc

SEQ ID NO: 142 - DNA, 102 bp, Arabidopsis thaliana
taacacttgatggagaagcagggcacgtgcgaacacaaatgaaatcgatcggtacttgttgatcatattttcgcacgtgttctactactccaacacgtgtct

SEQ ID NO: 143 - DNA, 193 bp, Brachypodium distachyon
gagtggacatatgaggcgaggcgcgcgaggtggagaagcagggcacgtgcattcgttccagctcgccgccggtgtgccgcgccgcggcctgggcgcgcgtgtcgcccgagcggccggccatgcgcgtgtgtgtgcatgcatgtgcccttcttctccaccgcgcaagcctcgcctgcgaacgcgcgcgcacgcc

SEQ ID NO: 144 - DNA, 209 bp, Brachypodium distachyon
aagagaggagagctcagcgagaaggaccgcgttggagaagcagggcacgtgcatgcatgcaactagagcttagctagtgtcaattcttccatctcttgtgcctggccgggatcgatcttatataccgagcttaatttggcacctattggatgctgcatgcatgtgttcttctcctccatcacggtcttcatttctctcgctttctttac

SEQ ID NO: 145 - DNA, 138 bp, Brachypodium distachyon
atgatttgaggagagggggcgagcaaaccttgctggagaagcagggcacgtgcttgaccggtcggccggagccgggttgcagcatgtgcgctccttctccagcatggcttctcgccccgtcgaccggcgttcggcagg

SEQ ID NO: 146 - DNA, 186 bp, Brachypodium distachyon
agcgagaaggaccgcgttggagaagcagggcacgtgcatgcatgcaactagagcttagctagtgtcaattcttccatctcttgtgcctggccgggatcgatcttatataccgagcttaatttggcacctattggatgctgcatgcatgtgttcttctcctccatcacggtcttcatttctctcgct

SEQ ID NO: 147 - DNA, 127 bp, Brachypodium distachyon
tggtgagaatgcccctgttggagaagcagggcacgtgcaaagatcggagcacgtttcgtatgtgatgtacgtatgtgcatggatcgctgtgcacgcgctccccttctccaccatggccttcttacca

SEQ ID NO: 148 - DNA, 161 bp, Brachypodium distachyon
aggcgaggcttgcgcggtggagaagaagggcacatgcatgcacacacacgcgcatggccggccgctcgggcgacacgcgcgcccaggccgcggcgcggcacaccggcggcgagctggaacgaatgcacgtgccctgcttctccacctcgcgcgcctcgcct

SEQ ID NO: 149 - DNA, 86 bp, Brassica napus
ccacgttggagaagcagggcacgtgcaaaccaacaaacacgagatctatctcatgtaatttgcacgtgctccactcctccaacatg

SEQ ID NO: 150 - DNA, 92 bp, Brassica rapa
cctccacgttggagaagcagggcacgtgcaaaccaacaaacacgagatctatctcatgtaatttgcacgtgctccactcctccaacatgagc

SEQ ID NO: 151 - DNA, 278 bp, Citrus sinensis
gtgtagagcaagatggagaagcagggcacgtgcattactaactcaaccgcacatacatctactaacaaaaattaattaatccacacattttgaagaaaccaaatcaagaagcagcaagctctttctgttgttatagctcagtcatgagttgagttatttcttcatgtgcccttcttccccatcatgaccgcggcaccttgtgtgtgtgtgtgtgtgtctgtgtatcatcagggatcagctcaagatctgaatttcttcttagcaaaattcatgtgcca

SEQ ID NO: 152 - DNA, 297 bp, Citrus trifoliata
gaaggtgtgtgtagagcaagatggagaagcagggcacgtgcattactaactcaaccgcacatacatctactaacaaaaattaattaatccacacattttgaagaaaccaaatcaagaagcaggaggctctttctgttgttatagctcagtcatgagttgagttatttcttcatgtgcccttcttccccatcatgaccgcggcaccttgtgcgtgtgtgtgtgtgtatcatcagggatcagctcaagatctgaatttcttcttagcaaaattcatgtgccaatctaaagcctaccaaa

SEQ ID NO: 153 - DNA, 102 bp, Festuca arundinacea
ggtagctgctgttgttgctggcactgttattattgaagctggagaagcagggcacttgctcaagtgcagtggcggcagaggtggagttcatggtggcatgga

SEQ ID NO: 154 - DNA, 102 bp, Festuca arundinacea; identical to SEQ ID NO: 153

SEQ ID NO: 155 - DNA, 101 bp, Gossypium hirsutum
ggggtgagtatctcttgttggagaagcagggcacgtgcaagttcctatgtttaagtgaactttgcacgtgctccccttctccaccgtgagtttctcattct

SEQ ID NO: 156 - DNA, 99 bp, Medicago truncatula
cttgttggagaagcagggcacgtgcaaatcctctttctgattcattctctcataatgcatatcaatatcttttgcacgtgctccccttctccaactagg

SEQ ID NO: 157 - DNA, 144 bp, Medicago truncatula
caagatggagaagcagggcacgtgcaatactaactcatgaacatacacacatgatggaagtgaatacaaagagaatctacattctcttatgtttcatttttttaattgagttagttcttcatgtgcccctcttccccatcatga

SEQ ID NO: 158 - DNA, 86 bp, Medicago truncatula
cttgttggagaagcagggcacgtgcaaccgtagatctctctcaaacttcaatctcattttgcacgtgctccacttttccaacttga

SEQ ID NO: 159 - DNA, 72 bp, Medicago truncatula
cacgttggagaagcagggcacatgcttgattttatcaaagttctgagcatgtgctcttgctctccatcatgg

SEQ ID NO: 160 - DNA, 156 bp, Oryza sativa
gtgagaaggaccgcgttggagaagcagggcacgtgcatgcatatgttcatcatcatcttcttcctcctcctctagctccagccttgtgtgggttggaagtttagatagaactcgcactgcacgtggtctccttctccatcccggtctttttctcac

SEQ ID NO: 161 - DNA, 109 bp, Oryza sativa
gtgcacggtggagaagcagggcacgtgcattaccatccactcgcctgccggccgccggccgccattgccatggatggttcttcatgtgcccgtcttctccaccgagcac

SEQ ID NO: 162 - DNA, 119 bp, Oryza sativa
aggttcttgttggagaagcagggtacgtgcaaaatgcacaccggttggtcgagctaattaacaagctctgacgaccatggtgatcgaatgcacgtgctccccttctccaccatggcctt

SEQ ID NO: 163 - DNA, 94 bp, Oryza sativa
caaaccgtgctggagaagcagggcacgtgctcgacggcggggctggctggccggccggcttgcagcatgtgcgctccttctccagcatggcttc

SEQ ID NO: 164 - DNA, 132 bp, Oryza sativa
ttgtgcagggtggagaagcagggcacgtgagcggccatccagtgtagcttcgctgcgcgtccatggcggcgaacgcgcgtgatctggagtttggatggtcgttcatgtgtccgtcttctccaccgagcactg

SEQ ID NO: 165 - DNA, 210 bp, Oryza sativa
tgaggatggcgaggcgcgcgaggtggagaagcagggcacgtgcattcctagagcttccgtccagctccccggcgggctagctagctcactccgccgccgccgccgccgccgccggcgcgcgcacggctggctggctccggccggctgagatgcatgcacggatgcatgtgcccttcttctccaccgtgcacgcctcgcctgcagcaagga

SEQ ID NO: 166 - DNA, 85 bp, Populus trichocarpa
ggttccttgctggagaagcagggcacgtgcaaaatcctgatgaagtgcttacactttgcacgcgctcttcttctccaacacgggc

SEQ ID NO: 167 - DNA, 162 bp, Populus trichocarpa
gtgagcaagatggagaagcagggcacgtgcactactaactcatgcacacagagagggagacgcatttcttgctggagttacgagttacgactcttacctactattgattttgttagctccagtgagttagttattcatgtgcctgtcttcctcatcatgatc

SEQ ID NO: 168 - DNA, 82 bp, Populus trichocarpa
tagctcttgctggagaagcagggcacgtgcaagctctctcctcaagctttccttgcacgtgctccccttctccaacatgggt

SEQ ID NO: 169 - DNA, 99 bp, Populus trichocarpa
tggctcacgctggagaagcagggcacgtgcaaaatccttctcggcttccagatgctgatgaagcactctttgcacgtgctcccctcctccaacatgagt

SEQ ID NO: 170 - DNA, 155 bp, Populus trichocarpa
gtgagcaagatggagaagcagggcacgtgcattactaactcatgcacacagagtgagagagacatttcttgctggagttatgactcttacctactatagattgtgttggcttcagcgagttagttcttcatgtgcctgtcttccccatcatgatc

SEQ ID NO: 171 - DNA, 90 bp, Populus trichocarpa
tgagccatgctggagaagcagggcacatgctaaatctatcagcttgaaagtctgatagttttgcatgtgctctatctctccagcttggac

SEQ ID NO: 172 - DNA, 78 bp, Ricinus communis
cttgctggagaagcagggcacgtgcaattcctttctttccctctccgaattgcacgtgctccacttctccaacatggg

SEQ ID NO: 173 - DNA, 104 bp, Ricinus communis
cctgctggagaagcagggcacgtgcaaaccccaacactccttggtttcttgaatgccaatggaaactatactcttttgcacgtgctccccttctccaacatggt

SEQ ID NO: 174 - DNA, 116 bp, Ricinus communis
caagatggagaagcagggcacgtgcattactaactcatgcataattgaacatacatacatacgcatttttcttgctggaggttagttcttcatgtgcccgtcttccccatcatgac

SEQ ID NO: 175 - DNA, 85 bp, Ricinus communis
catgctggagaagcagggcacatgctagatttatcagaatgaaaaacctgagaattttgcatgtgcctttgttctccagcttgga

SEQ ID NO: 176 - DNA, 126 bp, Sorghum bicolor
tggagaagcagggcacgtgcattaccatccaatgccgccaagctcgatcctcctctgagcttgctagctccatcagctcgccagccatggctggatggatggttcttcatgtgcccatcttctcca

SEQ ID NO: 177 - DNA, 111 bp, Sorghum bicolor
gaggggcgagcaaaccgtgctggagaagcagggcacgtgctcgtcgtcgctgcatgcgtggtcgtcgtcgcagcacgtgcgctccttctccaacatggcttctcgccccca

SEQ ID NO: 178 - DNA, 153 bp, Sorghum bicolor
tatggtgtgtttgtgcagggtggagaagcaggacacgtgagcgaccatccagtttccatcgctggctctccgctgcgggcgctgccgtgcgttggatcgtcgttgggtggtcgctcatgtgtccgtcctctccaccgagcaccggtacatccg

SEQ ID NO: 179 - DNA, 160 bp, Sorghum bicolor
tggagaagcagggcacgtgcatgctcctaacttctcgcgcgctcctctccgccgagatccatcgttcatctatatctccatggatatggagctatatatggactggatcgcggagagaaagctagctagtatactgcacgcacgtgttctccttctccat

SEQ ID NO: 180 - DNA, 205 bp, Sorghum bicolor
gttggagaagcagggcacgtgcagagacacgccggccggagcacggccgccgccgatagatcgacctcgcactcgcactcgcacaccagctgcgtctgcgtgtggatgaggtcgagatcgtcgtcggtcgaagctagcgaggaacctcttgcttgttgcttcacccgctagctcctgcactgcacgtgctccccttctccaccat

SEQ ID NO: 181 - DNA, 156 bp, Triticum aestivum
ggtggagaagcagggcacgtgcatccatttccagctcggcattcccggcgtccggccggccggctgccgcggccttgcctggctgggtagtgcgtcgctcgatccggccgtgcgccggcggccggcccttgcatgcatgtgcctttcttctccacc

SEQ ID NO: 182 - DNA, 128 bp, Theobroma cacao
ccgggtgagtagctccttgttggagaagcagggcacgtgcaaaattgccaatccattttggcttctcaaatgccatgtctcgcgttctgcacgtgctccccttctccaacacgggttcctcgccctat

SEQ ID NO: 183 - DNA, 130 bp, Theobroma cacao
agtgggtgagtagctcttgttggagaagcagggcacgtgcaagtcctagtttagctatctcgagtttaacatactcccccagtgaactgtgcacgtgctccccttctccaccatgagttcctcacccttt

SEQ ID NO: 184 - DNA, 112 bp, Theobroma cacao
ggagagagggtaagccatgctggagaagcagggcacatgctaaagcaatcagacaaaaatctgagaaattttgcatgtgccctggctctccagcttggaccgtctcccttac

SEQ ID NO: 185 - DNA, 121 bp, Vitis vinifera
agctccttgttggagaagcagggcacgtgcagatttgcctcacttttcccccttttttcttctttactcccaccaccgcccacaggcttgcacgtgctccccttctccaacatgggttcct

SEQ ID NO: 186 - DNA, 93 bp, Vitis vinifera
taaaccatgctggagaagcagggcacatgctggatcaatcagcacctggatctgagattttacatgtgccctggctctcccacttggatccta

SEQ ID NO: 187 - DNA, 97 bp, Vitis vinifera
ttgagcaagatggagaagcagggcacgtgcattactagctcatgcaccacaaaccaataatcttttgctcgagttagactcttgcttgctgctttga

SEQ ID NO: 188 - DNA, 118 bp, Vitis vinifera
aagctcttgatggagaagcagggcacgtgcagttcacaaattctaatctgctctgcacgtgcagttcacaaattctaatctgctctgcacgtgctccccttctccaacatgggttcct

SEQ ID NO: 189 - DNA, 152 bp, Zea mays
cagtgacaaggaccacgctggagaagcagggcacgtgcatgcgcataccatatagctagacgatgttctctctcgctccgctcgaccaagcttcatgtatggatgggtacgcacgcacgtgttctccttctccatcgaggtctttctcactt

SEQ ID NO: 190 - DNA, 128 bp, Zea mays
tagacggtggctgtgcgtggtggagaagcagggcacgtgcattaccatccaatgccgccgggtgggtgggtggaatggatggatggttcttgatgtgcccatcttctccaccgagcacgaactgtctt

SEQ ID NO: 191 - DNA, 270 bp, Zea mays
tggcgaggtgcgcgcggtggagaagcagggcacgtgcattctttccgtcgccggccggcttggcagcggccggcggcccggctctcgcagtcacgcgtacgtcgcctgagcggcgcgcgcgagagagagagacacggcaggtcgtcgccggcgcggctaactggtgcaggtgcagcagctagcttctgaaacccagccagccagccagccggccggccggccgatcgatgcgtgcatgtgcccttcttctccatcgggcacgcctcgcct

SEQ ID NO: 192 - DNA, 216 bp, Zea mays
tgagtgagaaggaccacgctggagaagcagggcacgtgcatgcacatacgccattctcgatctctcctctccaccactactgcatctagctatctccatggatggatgtacgtagctcggactggatcgatcggagaagcaataagctagcgagctcatgcatgctggctgtgcacgcacgtggtctccttctccatcacggttctttctcacttc

SEQ ID NO: 193 - DNA, 116 bp, Zea mays
gtggagaagcaggacacgtgagcgaccatccagttccactcccgctggctcgctgctggagctcccggcggcttggtgagtttgggtggtcgttcatgtgtccgccctctccaccg

SEQ ID NO: 194 - DNA, 108 bp, Zea mays
ctggagaagcagggcacgtgctcgccgacaagtcgaagccttagggcagcttggcctgtggctgcacgcgtacgtggtcgtcgcagcacgtgcgctccttctccaaca

SEQ ID NO: 195 - DNA, 171 bp, Zea mays
ttggagaagcagggcacgtgcagagacacgccggagcacggccgccgccgatctaccgacctcccaaacctgccttatggtgtgggggtggaggtcgtcggtggaagcgatagctgtcgttgttgcttcgatgttgttagctcctcctgcacgtgctccccttctccacca

SEQ ID NO: 196 - DNA, 219 bp, Zea mays
gtggagaagcagggcacgtgtgaattcattcgttccatcgacggtctggcgccgactcacgcatcgccggcgcggcgagctgagcgcgtgacacggaaagcaggtccgccgcgccggcgcccgcgctggccgatcgaccggctaactgtagctccacgaagcaaagccaggccagccggccgatcgatgcgtgcgtgcatgtgcccttcttctccatcg

SEQ ID NO: 197 - DNA, 155 bp, Arabidopsis lyrata
gagtctcaccgtcgggctcggattcgcttggtgcaggtcgggaaccaattcggctgacacagcctcgtgactttaaaacctttattggtttgtgagcagggattggatcccgccttgcatcaactgaatcggatccccgaggtgtaagaaaactc

SEQ ID NO: 198 - DNA, 176 bp, Arabidopsis lyrata
agagagaaatacgatgatttaaagttaccggcggtctcggattcgcttggtgcaggtcgggaactgattagctgacaattacacgtgtgttgtcatggttggtttgtgagatcccgtcttgtatcaactgaatcggagtccgaggtgaaaaaaataaggatcttactttcagatct

SEQ ID NO: 199 - DNA, 117 bp, Aquilegia coerulea
ttggcttagtgcagctcggggactgatggcccaccagggccagcagcaaaaccaccaacacgaccagcactttgaatcccccgtggttgaactccttgagggggtactattccaaat

SEQ ID NO: 200 - DNA, 138 bp, Arabidopsis thaliana
caccatcgggctcggattcgcttggtgcaggtcgggaaccaattcggctgacacagcctcgtgacttttaaacctttattggtttgtgagcagggattggatcccgccttgcatcaactgaatcggatcctcgaggtg

SEQ ID NO: 201 - DNA, 124 bp, Arabidopsis thaliana
ttaccggcggtctcggattcgcttggtgcaggtcgggaactgattggctgacaccgacacgtgtcttgtcatggttggtttgtgagctcccgtcttgtatcaactgaatcggagtccgaggtga

SEQ ID NO: 202 - DNA, 136 bp, Brachypodium distachyon
tgctcctcccctgccgccgccgcctcgggctcgcttggtgcagatcgggaccctccgcccgcccccgccgggccggatcccgccttgcaccaagtgaatcggagccggcgcagcgacttttttttacttaattcct

SEQ ID NO: 203 - DNA, 133 bp, Brassica napus
ttacggcggcctctgactcgcttggtgcaggtcgggaactgattggctgacaccgccacgtggctttccatggttggcttgtgagcagggatcggatcccgccttgtatcaagtgaatccgagtccgacgtga

SEQ ID NO: 204 - DNA, 176 bp, Citrus clementine
gttaccggcggtctctaattcgcttggtgcaggtcgggaactgattggctagtttttttttgaaattttttgacagcgaggtggcgtgttgtaattggtcgtcacgtttaaattaatgattgaaaaaaatgcgaattggatcccgccttgcatcaactgaatcggagaccgaggtg

SEQ ID NO: 205 - DNA, 201 bp, Citrus trifoliata
agttaccggcggtctctaattcgcttggtgcaggtcgggaactgattggctagtttttttttgaaattttttgacagcgaggtggcgtgttgtaattggtcgtcacgtttaaattaatgattgaaaaaaatgcgaattggatcccgccttgcatcaactgaatcggagaccgaggtgagagattggatggatatttacctt

SEQ ID NO: 206 - DNA, 106 bp, Hordeum vulgare
gtccgtcgccgccgcctcgggctcgcttggtgcagatcgggaccctccgcccgccccgacgggccggatcccgccttgcaccaagtgaatcggagccggcgcagcg

SEQ ID NO: 207 - DNA, 98 bp, Medicago truncatula
ctgatttgcttggtgctggtcgggaaccattacatccacagtttccgtagaaactttcccttagtaaattggaccccgtcttgcatcaatcgaatcag

SEQ ID NO: 208 - DNA, 87 bp, Oryza sativa
cgcctcgggctcgcttggtgcagatcgggacccgccgccgccgctgccggggccggatcccgccttgcaccaagtgaatcggagccg

SEQ ID NO: 209 - DNA, 106 bp, Oryza sativa
tggtcttgtgaggcttggtgcagctcgggaactgttcttgatggactggcaggaactccatgtccaccactgccactcctgtgttgtggcattcctccttgccgtt

SEQ ID NO: 210 - DNA, 160 bp, Populus trichocarpa
ggtctctgattcgcttggtgcaggtcgggaactgattcggcgatttgattgccagatggctcgacatgactggttgttgtggaaaaagaaaaggaaggaaacaggaaaaaaaacaaagaatagcgaattggatcccgccttgcatcaactgaatcggagg

SEQ ID NO: 211 - DNA, 136 bp, Populus trichocarpa
ggtctctaattcgcttggtgcaggtcgggaactgattcggcgatttgattgccagatggctaaacacgattggctgtgaggcaaattataaaaagaaagagaattggatcccgccttgcatcaactgaatcggaga

SEQ ID NO: 212 - DNA, 131 bp, Ricinus communis
ctaattcgcttggtgcaggtcgggaactgattggcttttctatttctaatgatttccaggcggcgaaaagttattggtcaagattagatggaaagtgaattggatcccgccttgcatcaactgaatcgaag

SEQ ID NO: 213 - DNA, 106 bp, Sorghum bicolor
gccgccgcgccgcctcgggctcgcttggtgcagatcgggacctgccgccgtgctcggacgggacagatcccgccttgcaccaagtgaatccgagccggagcagccg

SEQ ID NO: 214 - DNA, 104 bp, Saccharum officinarum
gccgccgcgccgcctcgggctcgcttggtgcagatcgggacccgccgcccggccgacgggacggatcccgccttgcaccaagtgaatcggagccggcgcagcca

SEQ ID NO: 215 - DNA, 103 bp, Saccharum officinarum
ccgccccgcgccgtctgggctcgcttgggcagatcgggacccgccgcccggccgacgggacggatcccgccttgcaccaagtgaatcggagccggcgcagcca

SEQ ID NO: 216 - DNA, 99 bp, Saccharum spp.
gccgcgccgcctcgggctcgcttggtgcagatcgggacccgccgcccggccgacgggacggatcccgccttgcaccaagtgaatcggagccggcgcagc

SEQ ID NO: 217 - DNA, 167 bp, Theobroma cacao
agttaccggcggtctctaattcgcttggtgcaggtcgggaactgattggctgctgctccttaatagtggattggattggatttaggccagatggcgggccgtgactggaggaggcacgaattggatcccgccttgcatcaactgaatcggagaccgcggtgaggggt

SEQ ID NO: 218 - DNA, 132 bp, Vitis vinifera
ggtctctaattcgcttggtgcaggtcgggaaccgacttcgccgctccggcagcgccggaggcacgcggcggcctacgattggttgctgagcgaattccgatcccgccttgcatcaactgaatcggagacggc

SEQ ID NO: 219 - DNA, 104 bp, Zea mays
gaagccgcgccgcctcgggctcgcttggtgcagatcgggacccgccgcccggccgacgggacggatcccgccttgcaccaagtgaatcggagccggcggagcga

SEQ ID NO: 220 - DNA, 104 bp, Zea mays
tccgccgcgccgtctcgggctcgcttggtgcagatcgggacccgccgcccggccgacgggacggatcccgccttgcatcaagtgaatcggagccggcgcagcga

SEQ ID NO: 221 - DNA, 126 bp, Arabidopsis lyrata
tcctggatttgaatgaacatcattgagtgcagcgttgatgtaatttcctttttttttcattgttgaatggaattaaaagaatttacaccggcgttgcgctcaattatgtttttcttattttcagga

SEQ ID NO: 222 - DNA, 107 bp, Arabidopsis thaliana
tgaatgaacatcattgagtgcagcgttgatgtaatttcgttttgtttttcattgttgaatggattaaaagaatttataccagcgttgcgctcaattatgtttttcta

SEQ ID NO: 223 - DNA, 124 bp, Brachypodium distachyon
gaagaggcgcaaaggcatcattgagtgcagcgttgatgaacaggggccaggcgaccggcggccggtccggttcggttcaccggcgctgcacacagtgacgcccttgcattctctggcccgattc

SEQ ID NO: 224 - DNA, 292 bp, Brachypodium distachyon
gcgaagaggaattagaagaggcgcaaaggcatcattgagtgcagcgttgatgaacaggggccaggcgaccggcggccggtccggttcggttcaccggcgctgcacacagtgacgcccttgcattctctggcccgattcatttgtgtgcgaagaggaattagaagaggcgcaaaggcatcattgagtgcagcgttgatgaacaggggccaggcgaccggcggccggtccggttcggttcaccggcgctgcacacagtgacgcccttgcattctctggcccgattcatttgtgt

SEQ ID NO: 225 - DNA, 96 bp, Brassica napus
gaacatcattgagtgcagcgttgatgtgatttacttctctttttcattgttgaatggattaaagcaatttacatcaacgttggctcaattatgttt

SEQ ID NO: 226 - DNA, 96 bp, Brassica napus
gaacatcattgagtgcagcgttgatgtgatttacttatctttttcattgttgaatggattaaagcaatttacatcaacgttggctcaattatgttt

SEQ ID NO: 227 - DNA, 156 bp, Citrus sinensis
ccagattgaaaaaaacatcattgagtgcagcgttgatgaatatttgtcggtgctttgccaatatttctctatactagccaggattctttcaccagcgctgcactcgatcatgttttttagctctgctggttcaggattatccagataatacgcaca

SEQ ID NO: 228 - DNA, 113 bp, Hordeum vulgare
cgcagaggtgccgttgagtgcagcgttgatgaaccgtccggccatggcccgtccgcctccaccgaggccggagcggttcaccggcgctgcacgcaatgacgcctctgctttct

SEQ ID NO: 229 - DNA, 114 bp, Oryza sativa
atcaaatgcatcattgagtgcagcgttgatgaacaacggtaaccggtccatgttgatgcgcatttggccggtgatctgatcatcatcagcgcttcactcaatcatgcgtttggc

SEQ ID NO: 230 - DNA, 134 bp, Picea abies
agccgtggcaaccacatcattgagtgcagcgttgacgatagaaattccaccaatagatttgataatatctatctatttgtggaatgccattgatattgttaaccctgcactcaatagtatgtttgccaatggct

SEQ ID NO: 231 - DNA, 120 bp, Populus trichocarpa
tggagaaccatcattgagtgcagcgttgatgaaatcctccattttgtgctattaaactgttaccaaccctttatggggcatggcatcatttcaccagcgctgcattcaatcatgtttttc

SEQ ID NO: 232 - DNA, 114 bp, Populus trichocarpa
taattatacaccattgagtgcagcgttgatgaaattctcttgttagcttacttagctattttctcacgatggcgtggaatcatttcaccagcgctgcattcaaccatgtttttc

SEQ ID NO: 233 - DNA, 71 bp, Populus trichocarpa
caagtttagttcattgagtggagctttgatgacaatttgttttaaaagctctactgtattcgaacaatatg

SEQ ID NO: 234 - DNA, 157 bp, Ricinus communis
aaacatcattgagtgcagcgttgatgaattcccctacatgacatatttctgttggttaattcattaccaatatcccttctttaaaaaaagaagtaaacctttttgggaatagatggttttggatgatttcaccagcgctgcattcaatcatgttttt

SEQ ID NO: 235 - DNA, 91 bp, Sorghum bicolor
tcattgagtgcagcgttgatgagccagctggccggccagccgtgcgtccgccgccggcgccggccacggctcaccggcgctgcactcaatt

SEQ ID NO: 236 - DNA, 116 bp, Solanum lycopersicum
cagagtacttagaaacatgattgagtgcagcgttgatgacatatttgagaagtcctgcatttgggccttcctcatttttcatcaacgctaaactcgatcatgtttttaattctctg

SEQ ID NO: 237 - DNA, 123 bp, Theobroma cacao
attcccagatggaagacacatcattgagtgcagcgttgatgaattctttcactttttgccaactctatctcagatgaattcgccagcgctgcactcaatcatgtgttttgctctttctggaaa

SEQ ID NO: 238 - DNA, 118 bp, Vitis vinifera
gaagaaaacatcattgagtgcagcgttgatgaaactgaagtattccatttttcagcttctttgaagtccggcaagatgggttctggttgattccattggcgctgcactcaatcatgtc

SEQ ID NO: 239 - DNA, 143 bp, Zea mays
gtcattgagcgcagcgttgatgatgcggagcatgtacgtcttcccgggcttcactttcagcttgaacgtgtctgcgtgcatgcatatcataagtatggatatgagttagtataaagaatcatagccgttagcgctcattaact

SEQ ID NO: 240 - DNA, 84 bp, Zea mays
atcattgagcgcagcgttgatgagccagccgccgtgcctcccctgtcggctgcggcggctcaccagcgctgcactcaattacgc

SEQ ID NO: 241 - DNA, 122 bp, Arachis hypogaea
gaagaagagatgacaaagaactgggaacaggcagagcatgaatggaactatcaatagacacattttgttcattgacgctcatgcactgcctcttccctggctctctctttctttttccttct

SEQ ID NO: 242 - DNA, 211 bp, Arabidopsis lyrata
aaaggttagattggtattgcaatgaaagaagtggtaatgatagagagagacagggaacaagcagagcatggattgagtttactaaaacattaaacgactctgttttgtctctacccatgcactgcctcttccctggctccctcttttctctctctctctctctctctccctctctctatttctttctctcctttcatttcaatggattttt

SEQ ID NO: 243 - DNA, 218 bp, Arabidopsis thaliana
aaggttagattggtattgcaatgaaagaagacaaagcggtaatgagagagagacagggaacaagcagagcatggattgagtttactaaaacattaaacgactctgttttgtctctacccatgcactgcctcttccctggctccctctttttttctctatatttctctctctcctttcatttcacagctttcaatggaattttattgctactgctaacg

SEQ ID NO: 244 - DNA, 153 bp, Citrus sinensis
aggtaataataactctttggtgtgtgcgtgagagagaggcaagaagcaagagacaaaagacggggaacaggcagagcatggatggaaccattaacaggttctctgttttggctcctcccatgcactgcctcttccctggctctctgccttcct

SEQ ID NO: 245 - DNA, 213 bp, Oryza sativa
gggagttctgtgattggagaggagaggagacagggatgaggcagagcatgggatggggctatcaacagatgtagattattccttgcacaagagatgatgatgagctgtgaatgagttctgagagatggctggtgttgttgttgctccctcccctgcactgcctcttccctggctcccctgcacacctctctctctctctctctctctctgtgt

SEQ ID NO: 246 - DNA, 148 bp, Physcomitrella patens
acaaccagcactgcactgcatcttccctgtgccatcttcgtcgattgtctcgtaccgaaagtgattcaaccacagcgagatcagactcaactgttaaagctgatgacactgatacgagatgatgaagacataggcagggcagccagtg

SEQ ID NO: 247 - DNA, 144 bp, Physcomitrella patens
gtggaagagagagagtggtggaagggagggaagccagcgtgaggcaatgcatgacaacagcatgcccaggaggtcctgagggtgttgtcctcatgcactgcctcttccctggcttccctacatagctcgccattcttgtgctct

SEQ ID NO: 248 - DNA, 86 bp, Pinus taeda
gagacagggacgagttagggcatgggagttgcatatgcagaaacgtctgcttctgccattcttatgcactgcctcttccctggctc

SEQ ID NO: 249 - DNA, 105 bp, Populus trichocarpa
agagacagatgaagacggggaacaggcagagcatggatggagctactaacagaagtacttgttttggctctacccatgcactgcctcttccctggcttgtggctc

SEQ ID NO: 250 - DNA, 92 bp, Ricinus communis
aaagactgggaacaggcagtgcatggatggggctactaacagaaaacatctgttttggctctacccctgcactgcctcttccctggcttccg

SEQ ID NO: 251 - DNA, 205 bp, Sorghum bicolor
acagggacgaggcagagcatgggatggggccatcaacaacaaaatttccaatttccgtttgcttgcccacaaaatggagggacttgtcaggagaggtatcaggcagagagaaggtgcctccctggtgaaatggtgatggcctgagagagctcagctggtgtcctggtgttgttgcttcctcccctgcactgcctcttccctggct

SEQ ID NO: 252 - DNA, 109 bp, Selaginella moellendorffii
atttagtagtagctagggagagacattgcatgatagctacaccaaagagcaacctcggctccttgtgtggctttgatgcactgcctcttccctggctgcggccaagtta

SEQ ID NO: 253 - DNA, 283 bp, Saccharum officinarum
agaagatgggtatggttggagacagggatgaggcagagcatgggatgaggccatcaacaaaatttccaatttctgtcctccgctaggccgctactgcatttctgtttgcttgctcacaaaacggagggatttgtgagagttatcaggcagaaagaacaaagaaggtgcctccctggtgaagtggtgatggcctgacctgagacggctgagagctcagctggtgtcctgttgttgcttcctcccctgcactgcctcttccctggctccccaccgttgcccttgc

SEQ ID NO: 254 - DNA, 286 bp, Saccharum officinarum
agaagatgggtatggttggagacagggatgaggcagagcatgggatggggccatcaacaaaatttccaatttctgtcctccgctaggccgctactgcatttatgtttgcttgctcacaaaacggagggatttgtgagagttatcaggcagaaagaacaaagaaggtgcctccctggtgaagtggtgatggcctgacctgagagggctgagagctcagctggtgtcctggtgttgttgcttcctcccctgcactgcctcttccctggctccccaccgttgcccttgc

SEQ ID NO: 255 - DNA, 286 bp, Saccharum officinarum
agaagatgggtatggttggagacagggatgaggcagagcatgggatggggccatcaacaaaatttccaatttctgtcctccgctaggccgctactgcatttctgtttgcttgctcacaaaacggagggatttgtgagagttatcaggcagaaagaacaaagaaggtgcctccctggtgaagtggtgatggcctgacctgagagggctgagagctcagctggtgtcctggtgttgttgcttcctcccctgcactgcctcttccctggctccccaccgttgcccttgc

SEQ ID NO: 256 - DNA, 215 bp, Saccharum officinarum
ggaaggtatgtttgattggagacagggacgaggcagagcatgggatggggccatcaacaaaatttccaatttccgtttgcttgctcacaaaacgaaggtgcctgcctccctggtgatggcctgacctgagagggctgagagctcagctggtgtcctggtgttgttgcttccacccctgcactgcctcttccctggctccccaccgttgcccttgc

SEQ ID NO: 257 - DNA, 248 bp, Saccharum spp.
gagacagggatgaggcagagcatgggatgaggccatcaacaaaatttccaatttctgtcctccgctaggccgctactgcatttctgtttgcttgctcacaaaacggagggatttgtgagagttatcaggcagaaagaacaaagaaggtgcctccctggtgaagtggtgatggcctgacctgagacggctgagagctcagctggtgtcctgttgttgcttcctcccctgcactgcctcttccctggctc

SEQ ID NO: 258 - DNA, 180 bp, Saccharum spp.
gagacagggacgaggcagagcatgggatggggccatcaacaaaatttccaatttccgtttgcttgctcacaaaacgaaggtgcctgcctccctggtgatggcctgacctgagagggctgagagctcagctggtgtcctggtgttgttgcttccacccctgcactgcctcttccctggctc

SEQ ID NO: 259 - DNA, 187 bp, Triticum aestivum
attttgtgagtggagaggggggaggagacagggatggagcagagcaagggatgaggcaagcaacaaaatttaccacctgattatgagaagagggagagagttgccagagcttctgttgctgttgttgctccctccctgcactgcctcttccctggctcccctcccaaatctctccctccccctctct

SEQ ID NO: 260 - DNA, 107 bp, Vitis vinifera
aagaggaagacggggacgaggtagtgcatggatggaactattaacagaagaatgttaagctgtttttgctctacccatgcactgcctcttccctggctctgtctctc

SEQ ID NO: 261 - DNA, 191 bp, Zea mays
ggggttggttttgatttggagacagggatgagacagagcatgggatggggccatcaacaaagtggagggactagcttgcgaggcagaaagaaggtgccagtgccggtgcctccccggtgaaacgatgatgggagtgttgttgctccctcccctgcactgcctcttccctggctccgatcccccaccgttgc

SEQ ID NO: 262 - DNA, 147 bp, Zea mays
gacagggacgaggcagagcatgggtagggggccatcaacagaattccaaatttgatttctgtttgctcgctcacaaaatggagggactcaccacaaacacactcaggcgttgttgctccctcccctgcactgcctcttccctggctc

SEQ ID NO: 263 - protein, 299 aa, Phaseolus vulgaris
MSNISMVEAKLPPGFRFHPRDEELVCDYLMKKLTHNDSLLMIDVDLNKCEPWDIPETACVGGKDWYFYTQRDRKYATGLRTNRATASGYWKATGKDRPILRKGTLVGMRKTLVFYQGRAPKGRKTEWVMHEFRIEGPHGPPKVSSSKEDWVLCRVFYKSREVSAKPSMGSCYEDTGSSSLPALMDSYISFDQTQAHADEFEQVPCFSIFSQNQANPIFNHMTTMEPKLPATTYGGAPNLGYCLDPLSCDRKVLKAVLSQITKMERNPLNQSLKGSTSFGEGSSESYLSEVGMPHMWNNY

SEQ ID NO: 264 - protein, 303 aa, Glycine max
MSNISMVEAKLPPGFRFHPRDEELVCDYLMKKVAHNDSLLMINVDLNKCEPWDIPETACVGGKEWYFYTQRDRKYATGLRTNRATASGYWKATGKDRSILRKGTLVGMRKTLVFYQGRAPKGNKTEWVMHEFRIEGPHGPPKISSSKEDWVLCRVFYKNREVSAKPRMGSCYEDTGSSSLPALMDSYISFDQTQTHADEFEQVPCFSIFSQNQTSPIFNHMATMEPKLPANHATNAYGGAPNLGYCLDPLSCDRKMLKAVLNQITKMERNPLNQSLKGSPSLGEGSSESYLSEVGMPHMWNNY

SEQ ID NO: 265 - protein, 302 aa, Glycine max
MSNISMVEAKLPPGFRFHPRDEELVCDYLMKKVQHNDSLLLIDVDLNKCEPWDIPETACVGGKEWYFYTQRDRKYATGLRTNRATASGYWKATGKDRPILRKGTHVGMRKTLVFYQGRAPKGRKTEWVMHEFRIEGPHGPPKISSSKEDWVLCRVFYKNSEVLAKPSMGSCYEDTGSSTLPALMDSYISFDQTQTHADEFEQVPCFSIFSQNQTNPIFNHMTTMEPKFPLNHATTTYGGAPNLGYCLDPLSCDRKMLKAVLNQITKMERNPLNQSLKGSPSLGEGSSESYLSEVGMPHVWNY

SEQ ID NO: 266 - protein, 302 aa, Glycine max
MSNISMVEAKLPPGFRFHPRDEELVCDYLMKKVQHNDSLLLIDVDLNKCEPWDIPETACVGGKEWYFYTQRDRKYATGLRTNRATASGYWKATGKDRPILRKGTHVGMRKTLVFYQGRAPKGRKTEWVMHEFRIEGPHGPPKISSSKEDWVLCRVFYKNSEVLAKPSMGSCYEDTGSSTLPALMDSYISFDQTQTHADEFEQVPCFSIFSQNQTNPIFNHMTTMEPKFPLNHATTAYGGAPNLGYCLDPLSCDRKMLKAVLNQITKMERNPLNQSLKGSPSLGEGSSESYLSEVGMPHVWNY

SEQ ID NO: 267 - protein, 306 aa, Medicago truncatula
MSNISMVEAKLPPGFRFHPRDEELVCDYLMKKVTHSDSFLMIDVDLNKCEPWDIPEAACVGGKEWYFYTQRDRKYATGLRTNRATASGYWKATGKDRAILRKGTLVGMRKTLVFYQGRAPKGRKTEWVMHEFRIEGPHGPPKISSSKEDWVLCRVFYKNREVATKPPSMGSCYDDTGSSSLPALMDSYISFDQAQFHTDEYEQVPCFSMFSQNQTNPIYNNITTNMEPKLPLANNNNASTFGGAPYSLDPLSCDRKVLKAVLSQLSKMERNPINDQNLKGSSPSLGEGSSESYLSEVGMPHMWNNF

SEQ ID NO: 268 - protein, 302 aa, Populus trichocarpa
MSNISFVEAKLPPGFRFHPRDEELVCDYLMKKASHCDSLLMIEVDLNKCEPWDIPETACVGGKEWYFYSQRDRKYATGLRTNRATASGYWKATGKDRHILRKGTLVGMRKTLVFYQGRAPKGKKTDWVMHEFRLEGPLGQPKTSSEKEDWVLCRVFYKNTREVVAKPSIRSCYDDTGSSSLPALMDSYITFDQTQPNLDEHEQVPCFSIFSQIQTNQNFPYITQMEVPNLPTKGTGPFGQVPMNITTHSDAFSCDTKVLKAVLNHFNMMESNANIKGSPSLGEGSSESYLSDVGMPNLWNHY

SEQ ID NO: 269 - protein, 316 aa, Ricinus communis
MSNISMVEAKLPPGFRFHPRDEELVCDYLMKKITHSDSLLLIEVDLNKSEPWDIPETACVGGKEWYFYSQRDRKYATGVRTNRATASGYWKATGKDRPVLRKGTLVGMRKTLVFYQGRAPKGRKSDWVMHEFRLEGPLGPPQIPQQKEDWVLCRVFYKNREVAAKPSMGSCYDDTGSSSLPPLMDSFITFDQTQPNLDEYYDEQVSCFSIFNQNQNNLIFPHINQTDSNIHTKSSTPSAFGQLIPMTTTTTTTTNTTSYPNLETLSCDKKVFKAVLNQLTKMENNPGSMHGSPSLGEGSSESYLSEVGMSNIWNHY

SEQ ID NO: 270 - protein, 304 aa, Populus trichocarpa
MSNISFVEAKLPPGFRFHPRDEELVCDYLMNKASQCCDSLLMIEVDLNKCEPWDIPAARVGGKEWYFYSQRDRKYATGLRTNRATASGYWKATGKDRHVLRKGTLVGMRKTLVFYQGRAPKGKRTDWVMHEFRLEGPLGPPKISSDKEDWVLCRVFYKSNREVVAKPSMESCNNDTGSSSLPALLDSYITYEQTQPNLDEHEQVPCFSIFSQNQTSQNLLAPYTTQMEAPNAPAKCTSPFGKVPMDITTPLDSFSCDTKVLKTVLNNLTKMESYGNLKGSPSLGEGSSESYISEVGMSSLWNHY

SEQ ID NO: 271 - protein, 302 aa, Glycine max; identical to SEQ ID NO: 265

SEQ ID NO: 272 - protein, 302 aa, Glycine max; identical to SEQ ID NO: 266

SEQ ID NO: 273 - protein, 303 aa, Glycine max; identical to SEQ ID NO: 264

SEQ ID NO: 274 - protein, 299 aa, Phaseolus vulgaris; identical to SEQ ID NO: 263

SEQ ID NO: 275 - protein, 306 aa, Medicago truncatula; identical to SEQ ID NO: 267

SEQ ID NO: 276 - protein, 316 aa, Ricinus communis; identical to SEQ ID NO: 269

SEQ ID NO: 277 - protein, 302 aa, Populus trichocarpa; identical to SEQ ID NO: 268

SEQ ID NO: 278 - protein, 304 aa, Populus trichocarpa; identical to SEQ ID NO: 270

SEQ ID NO: 279 - protein, 302 aa, Glycine max; identical to SEQ ID NO: 266

SEQ ID NO: 280 - protein, 302 aa, Glycine max; identical to SEQ ID NO: 265

SEQ ID NO: 281 - protein, 303 aa, Glycine max; identical to SEQ ID NO: 264

SEQ ID NO: 282 - protein, 299 aa, Phaseolus vulgaris; identical to SEQ ID NO: 263

SEQ ID NO: 283 - protein, 306 aa, Medicago truncatula; identical to SEQ ID NO: 267

SEQ ID NO: 284 - protein, 316 aa, Ricinus communis; identical to SEQ ID NO: 269

SEQ ID NO: 285 - protein, 302 aa, Populus trichocarpa; identical to SEQ ID NO: 268

SEQ ID NO: 286 - protein, 304 aa, Populus trichocarpa; identical to SEQ ID NO: 270

SEQ ID NO: 287 - protein, 450 aa, Glycine max
MVTCPGSIIIHFFLFSAPLLSVLWSCSSVSALKPRAFILPIEKDPTTLQYSTSIDMGTPPLTLDLVIDIRERFLWFECGNDYNSSTYYPVRCGTKKCKKAKGTACITCTNHPLKTGCTNNTCGVDPFNPFGEFFVSGDVGEDILSSLHSTSGARAPSTLHVPRFVSTCVYPDKFGVEGFLQGLAKGKKGVLGLARTAISLPTQLAAKYNLEPKFALCLPSTSKYNKLGDLFVGGGPYYLPPHDASKFLSYTPILTNPQSTGPIFDADPSSEYFIDVKSIKLDGKIVNVNTSLLSIDRQGNGGCKLSTVVPYTKFHTSIYQPLVNDFVKQAALRKIKRVTSVAPFGACFDSRTIGKTVTGPNVPTIDLVLKGGVQWRIYGANSMVKVSKNVLCLGFVDGGLEPGSPIATSIVIGGYQMEDNLLEFDLVSSKLGFSSSLLLHMASCSHFRLV

SEQ ID NO: 288 - protein, 589 aa, Glycine max
MEPAKTIHNNVKYSPIFLAIFVLILASALSSANAKIHEHEFVVEATPVKRLCKTHNSITVNGQYPGPTLEINNGDTLVVKVTNKARYNVTIHWHGVRQMRTGWADGPEFVTSVPDCPGGSYTYRFTVQGQEGTLWWHAHSSWLRATVYGALIIRPREGEPYPFPKPKRETPILLGEWWDANPIDVVRQATRTGGAPNVSDAYTINGQPGDLYKCSSKDTTIVPIHAGETNLLRVINAALNQPLFFTVANHKLTVVGADASYLKPFTTKVLILGPGQTTDVLITGDQPPSRYYMAARAYQSAQNAAFDNTTTTAILEYKSPNHHNKHSHHRAKGVKNKTKPIMPPLPAYNDTNAVTSFSKSFRSPRKVEVPTEIDQSLFFTVGLGIKKCPKNFGPKRCQGPINGTRFTASMNNVSFVLPNNVSILQAHHLGIPGVFTTDFPGKPPVKFDYTGNVSRSLWQPVPGTKAHKLEFGSRVQIVLQDTSIVTPENHPIHLHGYDFYIVAEGFGNFDPKKDTAKFNLVDPPLRNTVAVPVNGWAVIRFVADNPGAWLLHCHLDVHIGWGLATVLLVENGVGKLQSIEPPPVDLPLC

SEQ ID NO: 289 - protein, 575 aa, Populus trichocarpa
MEVIKSIFADRHCSFFLVVLLLASTMSLAIAEIHHHDFVVQATKVKRLCKTHNSITVNGMFPGPTLEVKNGDTLVVKVVNKARYNVTIHWHGIRQMRTGWADGPEFVTQCPIRPGGSYTYRFNIEGQEGTLWWHAHSSWLRATVYGALIIHPREGSSYPFAKPKRETPILLGEWWDANPVDVVREATRTGAAPNISDAYTINGQPGDLYNCSSEDTTIVPIASGETNLLRVINAALNQPLFFTIANHKFTVIGADASYLKPFTTSVIMLGPGQTTDVLISGDQLPGRYYMAARAYQSAQNAPFDNTTTTAILEYKSALCPAKCTTKPVMPRLPAYNDTATVTAFSGSLRSPRKVEVPTDIDENLFFTIGLGLNNCPKNSRARRCQGPNGTRFTASMNNVSFVFPSNIALLQAYQQKVPGIYTTDFPAKPPVKFDYTGNVSRSLFQPVRGTKLYKLKYGSRVQIVLQDTSIVTPENHPIHLHGYDFYIIAEGFGNFNPKTHKSKFNLVDPPMRNTVAVPSNGWAVIRFVADNPGVWLMHCHLDVHITWGLAMAFLVEDGIGELQSVEPPPADLPIC

SEQ ID NO: 290 - protein, 577 aa, Vitis vinifera
MEALSCCIANSRSFLLGLLLLLASAVFFTEAETHHHDFVVQATPVKRLCKTHNTITVNGQYPGPTLEINNGDTLEVKVTNKARYNVTIHWHGIRQMRTGWADGPEFVTQCPIRPGGSYTYRFTVQGQEGTLWWHAHSSWLRATVYGALIIHPKPGSSYPFTKPKRETPILLGEWWDANPIDVVRQATRTGAAPNVSDAYTINGQPGDLYNCSSKDTVIVPIDSGETNLLRVINSGLNQELFFTVANHKFTVVSADASYTKPFTTSVIMLGPGQTTDVLITGDQPPARYYMAARAYQSAQGAPFDNTTTTAILEYKSAPCPAKKGVSTTPVFPSLPAFNDTATVTAFSKSFRSPAKVEVPTDIDESLFFTVGLGLNRCPPKFKSSQCQGPNGTRFTASMNNVSFVLPSNFSLLQAHQQGIPGVFTTDYPAAPPVKFDYTGNVSRSLWQPVPGTKLYKLKYGSRVQVVLQGTSIFTAENHPIHLHGYDFYIIAEGFGNFNPSTDTSKFNLVDPPLRNTVAVPVNGWAVIRFVADNPGVWLMHCHLDVHITWGLAMAFLVENGVGALQSIETPPADLPLC

SEQ ID NO: 291 - protein, 577 aa, Ricinus communis
MGDITNHIFANSCFLFFGLLLLLASTLSLANAKVHHHDFVVQATKVKRLCKTHNTITVNGMFPGPTIEVNSGDTLVVKVTNKARYNVTVHWHGIRQMRTGWADGPEFITQCPIRPGGSYTYRFTIEGQEGTLWWHAHSSWLRATVYGALIIYPKDGTSYPYAKPKRETPILLGEWWDANPIDVVREATRTGAAPNISDAYTINGQPGDLYNCSSKETVIVPIGSGETHLLRVINAALNQPLFFTIANHKFTVVGADALYLKPFSTSVIMLGPGQTTDVLISGDQPPARYYIAARAYQSAQNAPFDNTTTTAILEYKSAPCPAKCLTSKPIMPPLPAFNDTPTVTAFSKSLRSPRKVDVPTEIDENLFFTIGLGLNKCPKNFRARRCQGPNGTRFTSSMNNVSFVLPSNFSLLQAARQNIPGVFTTDFPAKPPVKFDYTGNVSQSLWQPVPGTKLYKLKYGSRVQIVLQDTSIVTPENHPIHLHGYDFYVIAEGFGNFNPKKDTAKFNLVDPPMRNTVAVPSNGWAVIRFVADNPGVWIMHCHLDVHITWGLAMAFLVEDGIGELQKLEPPPNDLPLC

SEQ ID NO: 292 - protein, 550 aa, Populus trichocarpa
MSLAIAKTHHHDFTVQATKVKRLCKTHNSITVNGMFPGPTLEVKNGDTLVVKVVNRARYNVTIHWHGIRQMRTGWADGPEFVTQCPIRPGGSYTYRFTIEGQEGTLWWHAHSSWLRATVYGALIIHPREGSSYPFSKPKRETPILLGEWWDANPIDVVREATRTGAAPNISDAYTINGQPGDLFNCSSKDTTIVPIDSGETNLLRVINAALNQPLFFTIANHKFTVVGADASYLKPFTTSVIMLGPGQTTDVLISGDQLPGRYYMAARAYQSAQNAPFDNTTTTAILEYKSVLCPAKCTKKPFMPPLPAYNDTATVTAFSRSFRSPRKVEVPTDIDENLFFTIGLGLNNCPKNFRARRCQGPNGTRFTASMNNVSFVFPSKASLLQAYKQKIPGVFTTDFPAKPQVKFDYTGNVSRSLFQPARGTKLYKLKYGSRVQIVLQDTSIVTPENHPIHLHGYDFYIIAEGFGNFNPKTDKSKFNLVDPPMRNTVAVPVNGWAVIRFVADNPGVWLMHCHLDVHITWGLAMAFLVEEGIGILQSVEPPPADLPIC

SEQ ID NO: 293 - protein, 576 aa, Vitis vinifera
MEALSCCIANSRSFLLGLLLLLASAVFFTEAETHHHDFVVQATPVKRLCKTHNTITVNGQYPGPTLEINNGDTLEVKVTNKARYNVTIHWHGIRQMRTGWADGPEFVTQCPIRPGGSYTYRFTVQGQEGTLWWHAHSSWLRATVYGALIIHPKPGSSYPFTKPKRETPILLGEWWDANPIDVVRQATRTGAAPNVSDAYTINGQPGDLYNCSSKDTVIVPIDSGETNLLRVINSGLNQELFFTVANHKFTVVSADASYTKPFTTSVIMLGPGQTTDVLITGDQPPARYYMAARAYQSAQGAPFDNTTTTAILEYKSAPCPAKKGVSTTPVFPSLPAFNDTATVTAFSKSFRSPAKVEVPTDIDESLFFTVGLGLNRCPPKFKSSQCQGPNGTRFTASMNNVSFVLPSNFSLLQAHQQGIPGVFTTDYPAAPPVKFDYTGNVSRSLWQPVPGTKLYKLKYGSRVQVVLQGTSIFTAENHPIHLHGYDFYIIAEGFGNFNPSTDTSKFNLVDPPLRNTVAVPVNGWAVIRFVADNPVWLMHCHLDVHITWGLAMAFLVENGVGALQSIETPPADLPLC

SEQ ID NO: 294 - protein, 574 aa, Populus trichocarpa
MEVINRIFANRHCSFFLLLLLASAMSLAIAKTHHHDFTVQATKVKRLCKTHNSITVNGMFPGPTLEVKNGDTLVVKVVNRARYNVTIHWHGIRQMRTGWADGPEFVTQCPIRPGGSYTYRFTIEGQEGTLWWHAHSSWLRATVYGALIIHPREGSSYPFSKPKRETPILLGEWWDTNPIDVVREATRTGAAPNISDAYTINGQPGDLFNCSSKDTTIVPIDSGETNLLRVINAALNQPLFFTIANHKFTVVGADASYLKPFTTSVIMLGPGQTTDVLISGDQLPGRYYMAARAYQSAQNAPFDNTTTTAILEYKSVLCPAKCTKKPFMPPLPAYNDTATVTAFSRSFRSPRKVEVPTDIDENLFFTIGLGLNNCPKNFRARRCQGPNGTRFTASMNNVSFVFPSKASLLQAYKQKIPGVFTTDFPAKPQVKFDYTGNVSRSLFQPARGTKLYKLKYGSRVQIVLQDTSIVTPENHPIHLHGYDFYIIAEGFGNFNPKTDKSKFNLVDPPMRNTVAVPVNGWAVIRFVADNPGVWLMHCHLDVHITWGLAMAFLVEEGIGILQSVEPPPADLPIC

SEQ ID NO: 295 - protein, 581 aa, Ricinus communis
MESLTHIFANHLLASFLGLLLVIASALSSANATPMTHNHEFVIQATSVKRLCKTQNVITVNGMFPGPTLEVNNGDTLVVTVTNRAQYNVTIHWHGIRQMRTGWADGPEFVTQCPIRPGGTYTYRFTIQAQEGTLWWHAHSSWLRATVYGALIIHPKEGSSYPFPKPKRETPIILGEWWNANPIDVLRKATRTGGAPNVSDAYTINGQPGDLYNCSSQDTVIVPIDSGETNLLRVINAAMNQPLFFTVANHRLTVVGADASYTKPFTTSVLMLGPGQTTDVLISGDQKPARYYMAARAYQSAQNAQFDNTTTTAILEYKSAPCAAKNCSSNKPIMPPLPAYNDTATVTAFSTSFRSRNKVLVPTEVDENLFFTVGLGLNTCPPNFNKSSQCQGPNGTRFAASMNNVSFQLPSNFSILQAHQLGIPRVFTTDFPASPPLKFDYTGNVSRSLWQAVAGTKVYKLKYGSRVQIVLQDTSIVTSENHPIHLHGYDFYIIAEGFGNFNPQTDTSKFNLVDPPLRNTVGVPVNGWAVIRFVADNPGVWLMHCHLDVHITWGLAMAFLVENGVGVLQSIEAPPEDLPPC

SEQ ID NO: 296 - protein, 581 aa, Arabidopsis lyrata
MEIVKSLIFISLAVVLLFASIAEANIKAHHHEFIIQATKVKRLCETHNSITVNGMFPGPMLVVNNGDTLVVKVINRARYNITIHWHGVRQMRTGWADGPEFVTQCPIRPGSSYTYRFTIQGQEGTLWWHAHSSWLRATVYGSLLVLPPAGSSYPFTNPHRNVPLLLGEWWDANPVDVLRESIRTGGAPNNSDAYTINGQPGDLYKCSSQDTTIVPINVGETILLRVINSALNQPLFFTVANHKLTVVGADASYLKPFTTNVIVLGPGQTTDVLITGDQPPNRYYMAARAYQSAQNAPFGNTTTTAILQYKSAPCCGVGGGSGTKKGISVKLIMPILPAYNDTNTVTRFSQSFRSLRRAEVPTEIDENLFVTVGLGLNNCPKNFRSRRCQGPNGTRFTASMNNISFALPSNYSLLQAHHHGIPGVFTTDFPAKPPVKFDYTGNNISRSLYQPDRGTKLYKLKYGSRVQIVLQDTGIVTPENHPIHLHGYDFYIIAEGFGNFNPKKDTAKFNLEDPPLRNTVGVPVNGWAVIRFVADNPGVWIMHCHLDAHISWGLAMAFLVENGNGVLQTMEQPPADLPVCY

SEQ ID NO: 297 - protein, 566 aa, Vitis vinifera
  MISC_FEATURE (68)..(68): X is any amino acid
MEALSCCIANSRSFLLGLLLLLASAVFFTEAETHHHDFVVQATPVKRLCKTHNTITVNGQYPGPTLEXNNGDTLEVKVTNKARYNVTIHWHGIRQMRTGWADGPEFVTQCPIRPGGSYTYRFTXQGQEGTLWWHAHSSWLRATVYGALIIHPKPGSSYPFTKPKRETPILLGEWWDANPIDVVRQATRTGAAPNVSDAYTINGQPGDLYNCSSKDTVIVPIDSGETNLLRVINSGLNQELFFTVANHKFTVVSADASYTKPFTTSVIMLGPGQTTDVLITGDQPPARYYMAARAYQSAQGAPFDNTTTTAILEYKSAPCPAKKGVSTTPVFPSLPAFNDTATVTAFSKSFRSPAKVEVPTDIDESLFFTVGLGLNRCPPKFKSSQCQGPNGTRFTASMNNVSFVLPSNFSLLQAHQQGIPGVFTTDYPAAPPVKFDYTGNVSRSLWQPVPEFQVVLQGTSIFTAENHPIHLHGYDFYIIAEGFGNFNPSTDTSKFNLVDPPLRNTVAVPVNGWAVIRFVADNPGVWLMHCHLDVHITWGLAMAFLVENGVGALQSIEXPPADLPLC

SEQ ID NO: 298 - protein, 121 aa, Glycine max
MALGRGSAVVLLLCFLLLHSQMARAATYTVGDSGGWTFNTVAWPKGKLFRAGDTLAFNYSPGTHNVVAVNKAGYDSCKTPRGAKVYKSGTDQIRLAKGQNYFICNYVGHCESGMKIAINAA

SEQ ID NO: 299 - protein, 121 aa, Medicago truncatula
MALGRASALVLLVCFFVLNSELAHAATYTVGGPGGWTFNTVGWPNGKRFRAGDTLVFNYSPSAHNVVAVNKGGYDSCKTPRGAKVYRSGKDQIRLARGQNYFICNFVGHCESGMKIAINAA

SEQ ID NO: 300 - protein, 121 aa, Glycine max
MALGRGSAVVLLLCFLVLQSEMARAATYRVGDSRGWTFNTVTWPQGKRFRAGDTLAFNYSPGAHNVVAVSKAGYDSCKTPRGAKVYRSGKDQIRLARGQNYFICNYVGHCESGMKIAINAA

SEQ ID NO: 301 - protein, 122 aa, Cicer arietinum
MALGRGSALVVLLVCFLVIHSELAQAAIYTVGGAGGWTFNTIAWPNGKNFKAGDTLVFNYSPGAHNVVAVSKAGYGSCKTPRGAKVYRSGKDQIRLARGQNYFICNYVGHCESGMKIAINAV

SEQ ID NO: 302 - protein, 125 aa, Populus trichocarpa
MVQGRGSAMVATVAVMLCMLLLHFDMAHAATYTVGGPGGWTFNVSGWPKGKSFKAGDILVFNYSTAAHNVVAVNKAGYSSCTSPRGAKVYTSGKDQIKLVKGQNFFICSFAGHCQSGMKIAVNAA

SEQ ID NO: 303 - protein, 124 aa, Glycine max
MSQGRGSASLPIVVTVVSLLCLLERANAATYSVGGPGGWTFNTNAWPNGKRFRAGDILIFNYDSTTHNVVAVDRSGYNSCKTPGGAKVFSSGKDQIKLARGQNYFICNYPGHCESGMKVAINAL

SEQ ID NO: 304 - protein, 126 aa, Ricinus communis
MAQGRGSANLAIATVVALLCLLTLTKQVRAATYTVGGSGGWTFNVDSWPKGKRFKAGDTLVFNYDSTVHNVVAVNKGSYTSCSAPAGAKVYTSGRDQIKLAKGQNFFICGISGHCQSGMKIAITAA

SEQ ID NO: 305 - protein, 124 aa, Glycine max; identical to SEQ ID NO: 303

SEQ ID NO: 306 - protein, 126 aa, Medicago truncatula
MTEGRGSASMNMVTLISLLCLLVLAESANAASYTVGGTGGWTYNTDTWPNGKKFKAGDVLSFNYDSTTHNVVAVDKSGYNNCKTPGGAKVFSSGSDQIRLSRGQNYFICSYPGHCQSGMKVSIYAV

SEQ ID NO: 307 - protein, 164 aa, Glycine max
MVLKTELCRFSGAKIYPGKGIRFVRGDSQVFLFANSKCKRYFHNRLKPSKLTWTAMYRKQHKKDIAQEAVKKRRRATKKPYSRSIVGATLEVIQKKRTEKPEVRDAAREAQLREIKERIKKTMDDKKAKKAEVAAKSQKSQGKGSISKGAMPKGPKLGGGGGKR

SEQ ID NO: 308 - protein, 163 aa, Populus trichocarpa
MVLKTELCRFSGAKIYPGKGIRFIRSDSQVFLFANSKCKRYFHNRLKPSKLTWTAMYRKQHKKDIAAETIKKRRRATKKPYSRSIVGATLEVIQKKRTEKPEVRDAAREAALREIKERIKKTKDEKRAKKAEVTAKVQKSSKGSVPKGAAPKGPKLGGGGGKR

SEQ ID NO: 309 - protein, 186 aa, Prunus avium
MVLKTELCRFSGAKIYPGKGIRFIRSDSQVFLFANSKCKRYFHNRLKPSKLTWTAMYRKQHKKDIAQEAVKKRRRTTKKPYSRSIVGATLEVIQKRRTEKPEVRDAAREAALREIKERIKKTKDEKKAKKAEVTKSQKSQGKGSIAKGGAQPKGPKLGGWRWQALSHCSVAYLLGSRVIDRTCLFV

SEQ ID NO: 310 - protein, 164 aa, Glycine max
MVLKTELCRFSGAKIYPGKGIRFVRGDSQVFLFANSKCKRYFHNRLKPSKLTWTAMYRKQHKKDIAQEAVRKRRRAAKKPYSRSIVGATLEVIQKKRAEKPEVRDAAREAALREIKERIKKTKDEKKAKKAEVASKSQKAGGKGNVSKGAMPKGPKLGGGGGKR

SEQ ID NO: 311 - protein, 164 aa, Cicer arietinum
MVLKTELCRFSGAKIYPGRGIRFIRGDSQVFLFVNSKCKRYFHNRLKPSKLTWTAMFRKQHKKDAAQEAVKKRRRATKKPYSRSIVGATLEVIQKKRTEKPEVRDAAREAALREIKERIKKTKDEKKAKKAEVASKAQKSQGKGNVQKGALPKGPKMGGGGGKA

SEQ ID NO: 312 - protein, 165 aa, Vitis vinifera
MVLKTELCRFSGAKIYPGKGIRFVRSDSQVFLFANSKCKRYFHNRLKPSKLTWTAMYRKQHKKDIAQEAVKKRRRATKKPYSRSIVGATLEVIQKRRTEKAEVRDAAREAALREIKERIKKTKDEKKAKKAEVMAKVQKTQGKGNVPKGAAAPKGPKIGGGGGKR

SEQ ID NO: 313 - protein, 163 aa, Glycine max
MVLKTELCRLSGAKIYPGKGIRFVRGDSQVFLFANSKCKRYFHNRLKPSKLTWTAMYRKQHKKDIAQEAVKKRRRAAKKPYSRSIVGATLEVIQKKRAEKPEVRDAAREAALREIKERIKKTKDEKKAKKAEVTAKSQKAGGKGISKGAMPKGPKLGGGGGKR

SEQ ID NO: 314 - protein, 163 aa, Medicago truncatula
MVLKTELCRFSGAKIYPGRGIRFIRSDSQVFLFVNSKCKRYFHNKLKPSKLTWTAMYRKQHKKDIAQEAVKKRRRATKKPYSRSIVGATLEVIQKKRSEKPEVRDAAREAALREIKERVKKTKDEKKAKKAESQAKAQKSVGKGNVSKGASKGPKLGGGGGKR

SEQ ID NO: 315 - protein, 178 aa, Camellia sinensis
MVLKTELCRFSGAKIYPGKGIRFIRSDSQVFLFSNSKCKRYFHNRLKPSKLTWTAMYRKQHKKDAAQEAVKKRRRTTKKPYSRSIVGATLEVIQKRRTEKPEVRDAAREAALREIKERIKKTKDEKKAKKAEVTAKTQKSQGKGISKGAVPKGPKLGGGGGKRCKAIFGCCVFYWGIC

SEQ ID NO: 316 - protein, 163 aa, Medicago truncatula
MVLKTELCRFSGAKIYPGRGIRFIRSDSQVFLFVNSKCKRYFHNKLKPSKLIWTAMYRKQHKKDIAQEAVKKRRRATKKPYSRSIVGATLEVIQKKRTEKPEVRDAAREAALREIKERIKKTKDEKKAKKAEVASKAQKSGKGNVQKGAMPKGPKMGGGGGKR

SEQ ID NO: 317 - DNA, 1206 bp, Phaseolus vulgaris
ctttatccattccaaatcatgagcaacataagcatggtggaggcaaagttgccaccagggttcaggttccatccaagagatgaagagcttgtctgtgattacttgatgaagaagctcacacacaatgattcccttctcatgatagatgttgaccttaacaagtgtgaaccttgggatattcctgaaacagcatgtgtgggagggaaagactggtatttctatacacaaagagaccgtaagtatgcaacagggttacgcacaaatcgtgccactgcctcagggtactggaaggccacagggaaggacagacctatccttcgcaagggcacccttgttggtatgaggaagactttggtgttctatcaaggaagggcacccaaagggagaaaaactgaatgggtcatgcatgagtttcgcatagaaggtccccatggaccccctaaagtttcttcttctaaggaagactgggttttgtgtagagtgttctacaaaagcagagaagtttcagccaaacctagcatgggtagctgctatgaagacacaggctcttcatctcttcctgcattaatggactcttacattagttttgatcaaactcaagctcatgcagatgagtttgagcaagtgccctgcttctccattttctctcagaaccaagcaaaccccattttcaaccacatgactaccatggagcctaaactacctgcaactacatatggaggagcaccaaatttgggttactgtttagaccctttatcctgtgacagaaaagtgttgaaagctgttttgagtcagatcacaaaaatggaaaggaatcctctgaaccaaagtcttaaagggtctacaagctttggagaaggaagctcagagagttacttatctgaagtggggatgcctcacatgtggaacaattactgatgtgggacttcagtccccaatgccaccatgccaaagaaaaattaaaacaataaaaaaaaatgaagatgaaattaatccattgtttgaagtgagtgatggtgagtgcatatgtttttacctgtggtgtttagctattactatgatacacatcctccattatgtgtcccaatatattaggaaaatgggacttgtaattcaaacttataaatatctgtagttaatttctagttagtggatgatttttttttcttgttgctgaaaaaaaaaaaaaaaaaaaaaaaaaaaaaa

SEQ ID NO: 318 - DNA, 973 bp, Glycine max
atatttcacctttattacctttatcgaaggaaaaatgagcaacataagcatggtagaggcaaagctgccaccagggttcaggtttcatccaagagatgaagaacttgtgtgtgattacttgatgaagaaggtggcacacaatgattcccttctcatgataaacgttgaccttaacaagtgtgagccatgggatattcctgaaacagcttgtgtgggagggaaggagtggtatttctatacacaaagagaccgtaagtatgcaacagggctacgcacaaatcgtgccactgcttcagggtattggaaggccacagggaaggacaggtctatcctccgcaagggcacccttgtagggatgaggaagactttggtgttctatcaagggagggcacccaaaggaaataaaactgagtgggtcatgcatgagtttcgaattgaaggacctcatggacctcctaaaatttcttcttccaaggaagattgggttttgtgtagggtgttctacaaaaacagagaagtttcagccaaacctagaatgggtagctgctatgaggacacaggatcttcatctcttcctgcattaatggactcttacataagttttgaccaaactcaaacccatgcagatgagtttgagcaagtgccctgcttctccattttctctcagaaccaaacaagccccattttcaaccacatggccactatggagcctaagttacctgccaaccatgcaactaatgcatatggaggagcaccaaatttgggttattgcttagaccctttatcatgtgacagaaaaatgttaaaagctgttttgaatcagatcacaaagatggaaaggaatccacttaaccaaagcctaaaagggtcaccaagcttaggagaaggaagttcagagagttacttatctgaagtggggatgccccacatgtggaacaattactgatgtgggacttcagtccttaaaccctct

SEQ ID NO: 319 - DNA, 976 bp, Glycine max
gattcatctcttatcttccttcacctttttattgaaagaaaaaaaaaatgagcaacataagcatggtagaggctaagctgccaccaggattcaggtttcatccaagagatgaagagcttgtgtgtgattacttgatgaagaaggtgcaacacaatgattcccttctcttgatagatgttgaccttaacaagtgtgagccatgggatattcctgaaacagcatgcgttggagggaaggagtggtatttctacacacaaagagaccgtaagtatgcaacagggttacgcacaaatcgtgccactgcctcagggtattggaaggccacagggaaggacaggcctatcctccgcaagggcacccatgtagggatgaggaagactttggtgttctatcaaggaagggcacccaaagggagaaaaactgagtgggtcatgcatgagtttcgtatcgaaggacctcatggacctcctaaaatttcttcttccaaggaagattgggttttgtgtagggtgttctacaaaaacagtgaagttttagccaaacctagcatgggtagctgctatgaggacacgggatcttcaactcttcctgcgttaatggactcttacataagttttgaccaaactcaaacccatgcagatgagtttgagcaagtgccctgcttctccattttctctcagaaccaaacaaaccccattttcaaccacatgaccactatggagcctaagttccctctcaaccatgcaactacaacatatggaggagcaccaaatttgggttattgcttagaccctttatcttgtgacagaaaaatgttaaaagctgttttgaatcaaatcacaaagatggaaaggaatccacttaaccaaagtctaaaagggtcaccaagcttaggagaaggcagttcagagagttatttatctgaagtggggatgccccacgtgtggaattactgatgtgagactttagtacgatc

SEQ ID NO: 320 - DNA, 1330 bp, Glycine max
ttttttttttttttttttttagcaaagaagaaaaaaaaatcatccactaaataaaattaactacaaatatttataagtttaaattacaagtcccattttcctaatatatagggacacatcatggatgggtatacattaatagctaaaacaccacaggtaaaacataagtactcaccatgacttaaaacaatggattgattttatcttcccccttatacctttttttttcttgtttttttttttaaaaaaaaaaatttcatggtggcataggtggaggagagggtttaagtactaaagtctcacatcagtaattccacacgtggggcatccccacttcagataaataactctctgaacttccttctcctaagcttggtgacccttttagactttggttaagtggattcctttccatctttgtgatttgattcaaaacagcttttaacatttttctgtcacaagataaagggtctaagcaataacccaaatttggtgctcctccatatgctgtagttgcatggttgagagggaacttaggctccatagtggtcatgtggttgaaaatggggtttgtttggttctgagagaaaatggagaagcagggcacttgctcaaactcatctgcatgggtttgagtttggtcaaaacttatgtaagagtccattaacgcaggaagagttgaagatcccgtgtcctcatagcagctacccatgctaggtttggctaaaacttcactgtttttgtagaacaccctacacaaaacccaatcttccttggaagaagaaattttaggaggtccatgaggtccttcgatacgaaactcatgcatgacccactcagtttttctccctttgggtgcccttccttgatagaacaccaaagtcttcctcatccctacatgggtgcccttgcggaggataggcctgtccttccctgtggccttccaataccctgaggcagtggcacgatttgtgcgtaaccctgttgcatacttacggtctctttgtgtgtagaaataccactccttccctccaacgcatgctgtttcaggaatatcccatggctcacacttgttaaggtcaacatctatcaagagaagggaatcattgtgttgcaccttcttcatcaagtaatcacacacaagctcttcatctcttggatgaaacctgaatcctggtggcagctttgcctctaccatgcttatgttgctcatttttttttttctttcaataaaaaggtgaaggaagataagagatgaagaaaaagagaaagataataataatagagaaaagaagttcaaagttgaagtgagaaattttgtgaggtgtcc

SEQ ID NO: 321 - DNA, 921 bp, Medicago truncatula
atgagcaacataagcatggttgaggcaaagctaccaccagggttcagatttcatcctagagatgaagaacttgtgtgtgattacttgatgaagaaggtgacacatagtgattcctttctcatgattgatgttgaccttaacaagtgtgagccatgggatattcctgaagcagcatgtgtgggagggaaagagtggtatttctacacacaaagagatcgtaaatatgcaacaggactacgaacaaatcgtgcaactgcatcaggttattggaaagcaacaggaaaggacagagctatccttcgtaagggcactcttgttggaatgagaaagactttagtgttctatcaaggaagggcacctaaagggagaaaaactgaatgggttatgcatgagtttcgtattgagggtcctcatggccctcctaaaatttcatcttctaaggaagattgggttttgtgtagggtgttctacaaaaacagagaagttgctacaaaacctccaagcatgggtagttgctatgatgacacaggctcttcatcacttccagcattaatggattcttacataagttttgaccaagctcaattccatacagatgaatatgagcaagtgccctgcttctccatgttttctcaaaaccaaaccaacccaatctacaacaatataacaaccaatatggaaccaaaattacctctagccaacaataacaatgcaagtacatttggaggagcaccttatagcttagaccctttatcatgtgatagaaaagtgttaaaagctgttttgagtcaactatcaaagatggaaagaaaccctattaatgatcaaaacctaaaagggtcatcaccaagcttaggtgaaggaagttctgagagttacttatctgaagtgggtatgccccacatgtggaacaatttctaa

SEQ ID NO: 322 - DNA, 1075 bp, Populus trichocarpa
gaggagatctcatattctatctccttgctctgttcgcattgttgctagcaatataaatacagtcttcccgttaccttgccttatctgttgagagcaagagagagatgagcaacataagctttgtggaggcaaaactgccaccagggtttaggttccatccaagagatgaagagcttgtatgtgattacttgatgaagaaggcttctcactgcgactcccttctcatgatagaggtcgacctcaacaagtgtgagccttgggatattcctgaaacggcatgcgtgggaggcaaggaatggtacttttacagccaaagagatcgtaaatatgcaactggactaagaactaatcgagcaacagcatctggatattggaaggccaccgggaaggacagacatatcctacgtaagggaacccttgttggcatgagaaagaccttggtgttctaccaaggtagggcacctaaagggaaaaaaaccgattgggtcatgcatgagtttcggcttgaagggccacttggtcagcccaaaacttcttcagagaaggaagattgggtcttatgtcgagtgttctataaaaacaccagagaagttgtggccaaacctagcataagaagctgctatgatgacacaggctcttcatctttgcctgcattaatggattcatacatcacttttgaccaaactcaacccaatttagatgagcacgagcaagtgccctgcttctccattttctcccaaatccaaaccaaccaaaatttcccttacataactcaaatggaagtaccaaatttacccacaaagggtacaggcccatttgggcaagtacctatgaatattaccactcattcagacgctttttcttgtgacacaaaggtactaaaagctgttttgaatcacttcaacatgatggaaagcaatgccaacattaaagggtcaccaagcttaggagaaggtagttcagaaagctacttatctgatgtgggcatgcccaacttatggaatcattattgatttttaggtcttaaatataggatttaaagagggtctctcttctcccactatcaaaaaaaaaa

SEQ ID NO: 323 - DNA, 951 bp, Ricinus communis
atgagcaacataagcatggtagaggcaaagttgccaccagggttcagattccacccgagagatgaagaactcgtctgtgattacttgatgaagaaaatcactcattctgattctcttctcttgattgaagttgacctcaacaagtctgaaccttgggatattcctgaaacagcatgtgtgggaggaaaagaatggtatttttacagccaaagagatcgtaaatatgcaacaggagtaaggacaaacagggcaacagcatctgggtattggaaggctacaggaaaagataggcctgtccttaggaagggaacccttgttggcatgaggaagaccttagtcttctatcaaggaagggcaccaaaaggaagaaaatcagattgggtcatgcatgagtttcgccttgaaggccctcttggtccccctcaaattcctcaacaaaaggaggattgggtcttatgcagagtgttctacaagaatagagaagttgcagccaaaccaagcatgggaagctgttatgatgacacaggctcttcatctctcccaccattgatggattctttcatcacttttgaccaaactcaacccaacttagatgagtattatgatgagcaagtgtcctgcttctccatttttaaccaaaaccaaaacaacctaattttcccacacatcaaccaaacagactcaaacattcacacaaaaagtagtactccaagtgcatttggacaactaatacccatgactactactacaacaactactactaatacaacttcttatcctaatttagagactctctcttgtgacaagaaggtatttaaggctgtcttgaatcaacttaccaagatggaaaacaatcctggaagcatgcatggatctccaagcttaggtgaagggagttcagaaagctacttatctgaagtgggtatgtccaatatatggaatcactattga

SEQ ID NO: 324 - DNA, 1006 bp, Populus trichocarpa
aaagggagaggaaaatgagcaacataagctttgtggaggcaaagttgccaccagggttcaggtttcatccaagagatgaagaacttgtatgcgattacttgatgaacaaggcttctcagtgctgtgattcccttcttatgatagaagttgacctcaacaagtgtgagccgtgggatatccctgcggcacgcgtaggtggcaaggaatggtatttttatagccaaagagatcgcaagtatgcaactggattgagaactaatcgtgcaacagcttctggatattggaaagccacagggaaggacaggcacgtccttcgcaagggcactcttgttggcatgagaaagaccttggtgttctaccaaggtagggcacccaaagggaaaagaactgactgggtcatgcatgagtttcgccttgaaggacctcttggtcccccaaaaatttcttcagataaggaagactgggttttatgccgagtgttctataaaagtaacagagaagttgtggccaaacctagcatggaaagctgcaataatgacacaggttcttcatctttgcctgcattattggattcatacatcacttatgagcaaactcaacccaatttagatgagcacgagcaagtgccctgcttctccattttctcacaaaaccaaaccagccaaaatctcctggctccgtacaccacccagatggaagccccgaacgccccggctaagtgtacgagcccatttggaaaagtacctatggatattaccactcccttggactctttttcttgtgacacaaaggtactaaaaactgttttgaataaccttaccaagatggaaagctatggcaaccttaaagggtcaccaagcttaggagaaggtagttcagagagctacatatctgaagtgggcatgtccagcttatggaatcattattgatttttaggtcttaaatataggatttaattagcgaagttcttcctctctcactgtcgaaaaagaggtaaaaaaaaaaa

SEQ ID NO: 325 - DNA, 976 bp, Glycine max; identical to SEQ ID NO: 319

SEQ ID NO: 326 - DNA, 1330 bp, Glycine max; identical to SEQ ID NO: 320

SEQ ID NO: 327 - DNA, 973 bp, Glycine max; identical to SEQ ID NO: 318

SEQ ID NO: 328 - DNA, 1206 bp, Phaseolus vulgaris; identical to SEQ ID NO: 317

SEQ ID NO: 329 - DNA, 921 bp, Medicago truncatula; identical to SEQ ID NO: 321

SEQ ID NO: 330 - DNA, 951 bp, Ricinus communis; identical to SEQ ID NO: 323

SEQ ID NO: 331 - DNA, 1075 bp, Populus trichocarpa; identical to SEQ ID NO: 322

SEQ ID NO: 332 - DNA, 1006 bp, Populus trichocarpa; identical to SEQ ID NO: 324

SEQ ID NO: 333 - DNA, 1330 bp, Glycine max; identical to SEQ ID NO: 320

SEQ ID NO: 334 - DNA, 976 bp, Glycine max; identical to SEQ ID NO: 319

SEQ ID NO: 335 - DNA, 973 bp, Glycine max; identical to SEQ ID NO: 318

SEQ ID NO: 336 - DNA, 1206 bp, Phaseolus vulgaris; identical to SEQ ID NO: 317

SEQ ID NO: 337 - DNA, 921 bp, Medicago truncatula; identical to SEQ ID NO: 321

SEQ ID NO: 338 - DNA, 951 bp, Ricinus communis; identical to SEQ ID NO: 323

SEQ ID NO: 339 - DNA, 1075 bp, Populus trichocarpa; identical to SEQ ID NO: 322

SEQ ID NO: 340 - DNA, 1006 bp, Populus trichocarpa; identical to SEQ ID NO: 324

SEQ ID NO: 341 - DNA, 1487 bp, Glycine max
gggagcaatacgagatcttaatttctttcacagatcacaatggttacttgtcctggttccataataatccatttcttcctcttctcagcgcctctcctttcagtcttatggtcttgttcatcagtttctgcattgaaaccccgtgccttcatcctccccatcgagaaagacccaaccacccttcagtactcaacttccatcgacatgggtacccctccactcacactagatctagtcatcgacatcagagaacgcttcctatggttcgagtgcggcaacgactacaactcctcaacctactaccctgtccgatgtgggactaagaaatgcaagaaagccaagggcacggcttgcattacatgcaccaaccaccctcttaaaacaggatgcaccaacaacacgtgtggtgtagatccattcaaccccttcggcgagttcttcgtgagtggagacgtgggtgaagacatcttgtcctcgctgcactcaacaagcggtgcacgagcaccttccactttgcacgtgccacgcttcgtctctacatgtgtgtacccagataaatttggggttgagggctttctccagggcctggctaagggcaagaaaggagttttaggccttgcaaggactgctatttccttaccaacacaactcgcagccaaatacaaccttgaacctaagtttgcactttgtttaccttcaacttcaaagtataataagcttggtgatctctttgttggtggtgggccttactatttgccacctcatgatgcttccaaatttctttcctacactccaattctcaccaacccccaaagcacaggtccaatctttgatgctgatccttccagcgagtactttattgacgtgaagtcaatcaagcttgacggtaaaattgttaacgttaacacctccctgctttctattgacagacagggaaatgggggctgcaaacttagtaccgtagttccttacaccaaattccacacttcaatataccagccacttgtgaatgactttgtaaagcaagcagcgcttaggaaaataaagagagtgacttcggtggcaccatttggggcgtgctttgattcaagaaccattggcaagaccgttactggacccaatgtgccgacaattgatctggttctcaaggggggagttcaatggagaatctatggtgccaattcaatggtcaaggtttctaagaatgtgctgtgccttggatttgtggatggaggtttggagccaggaagtcccattgcaacttcgattgtgattggtgggtatcagatggaggacaatcttttggagtttgatcttgtttcctcaaaacttggttttagctcctcccttttactccacatggctagctgttcccacttcagacttgtttgacttttcactttcgatcatttcagcaaagtttggttcatttggtgatgactgatgaataaattttatttgccattgtaaaaaaaaaaaaaaaaaaa

SEQ ID NO: 342 - DNA, 1770 bp, Glycine max
atggagcctgccaaaaccattcacaacaatgtcaaatactcccccatcttcttagccatctttgttctgatcttagcttcagcattgtcttcagcaaatgccaaaattcacgagcacgagtttgttgttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggcacggtgttaggcaaatgagaacagggtgggcagatggaccagaatttgtgacttcagtgcccgattgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcgcgagacacccattcttcttggggaatggtgggacgcaaaccctattgatgttgtgaggcaggccacacgaactgggggagccccaaacgtgtctgatgcatacactatcaatggtcaacctggtgatctttacaagtgctccagcaaagacaccaccattgtcccaatacatgccggcgagaccaaccttcttcgtgtcatcaatgctgcactcaatcaacctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttggagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtgcatggcttttgcattgtcacttggacgttcacattggatggggtttggctacggtgttgttggtggagaatggagttgggaagttgcaatccatagagcctcctcctgtggatcttcctctttgttag

SEQ ID NO: 343 - DNA, 1932 bp, Populus trichocarpa
aaaatggaggttatcaagtctatctttgctgatcgtcattgttctttcttcctggtcgtgctgcttctagcctcaacaatgtcattagcgattgcagagatccaccaccatgattttgttgttcaagcaacgaaagtgaagaggctgtgcaaaacccacaatagcatcacagtcaatgggatgttcccagggccaaccctggaagtgaaaaatggagacactctagttgtaaaagttgtcaacaaagcccgatacaacgttaccattcactggcatggtattaggcaaatgagaacgggctgggcagatgggccagaatttgtgacacaatgcccgatcagaccaggtgggagttatacctacaggtttaatattgaaggacaagaagggacactttggtggcatgctcatagctcttggcttagagccaccgtttatggtgctctaattatccatccaagagaaggatcctcgtatccgttcgctaagccaaagcgtgaaacacccatacttcttggcgaatggtgggatgcgaatcccgttgatgtggtgagggaggcaactagaactggagcagctccaaatatatctgatgcatataccattaatggtcaacctggtgatctttataattgttccagcgaagatactaccatcgttccgatcgcctctggtgagacaaacctccttcgagtcatcaatgctgcactcaatcaaccgcttttcttcacaatagccaatcacaagttcacagttattggtgctgatgcctcttatcttaaaccctttactacctcggtcataatgctaggaccaggccaaaccactgatgttttgatctcaggcgaccaactacctggccgatattacatggcagctcgagcttatcagagtgcgcaaaatgcaccatttgacaatactacaaccactgccattcttgaatacaaatctgccctttgccctgctaagtgtacaacaaaaccagttatgcctcgacttccagcttataacgacacagctacagttacagcattcagtggaagccttagaagccctcgaaaagtcgaggtcccaacagatattgacgagaacctcttcttcacaattggtctagggcttaacaactgtcctaaaaattccagagctaggcggtgtcaaggacccaacggcactcgcttcaccgctagcatgaacaatgtttcttttgtgtttccttcaaacattgcgttactgcaggcgtatcagcaaaaagttcctggaatttacaccactgactttccagcaaaaccaccagtaaagtttgattacactggtaatgtgagtcgttccctctttcagcctgttcgtggaactaagctgtacaagttgaagtacggatcaagggtgcagattgtgttgcaggacacgagcattgtcacaccagaaaaccacccaatccatcttcatggatacgacttctacatcattgcagaaggatttggaaatttcaatcccaaaactcataaatctaaatttaatcttgttgatccaccaatgaggaatacagttgcagtgccttcgaatggatgggcagtcattagatttgttgctgacaatccaggtgtgtggctaatgcactgtcacttggatgttcacatcacatggggtttggccatggctttcttggtagaggatgggattggggaattacagagtgtagagcctcctccagcagatctgccgatatgttaattatgatcgaaacataaaagaagattttacccattgttgacaaggattttctttctgttgttcttccctacatttggggacatagctcaatattgtatttttcctctctgcctttgaaacaagagtcgccatctcatgttagtgactgacctgcttgtttcttgattttagagctattcttctctcttcgttaataataca

SEQ ID NO: 344 - DNA, 2732 bp, Vitis vinifera
gaggccttgagctgctgcattgccaattctaggtctttcctcttgggccttttgcttctccttgcttctgcagtgttctttacagaagccgaaacccaccatcatgattttgttgtatgtcttctcttcttccttccttccttttacttgcttctttcctctcttggccttggattctgactatctgcattgatgtaacaggttcaagccaccccggtgaagaggctgtgcaaaacccacaacaccataacagtgaacgggcaatacccggggccaacgttggaaataaacaatggagacaccctagaagtcaaagtcaccaacaaagctcgatacaatgttaccattcactggtaattccatgcatgcatacatgtttcctctgtacaaaaatgaattgaattgaaggtatctggatagggcatgaatgcatgcaggcatgcaggcatgctcctacaaaagaaccaaccaaagtatatatttcatccaatgaacatactaatgtatatagaaagactcttcctaccttctatacttatcttactattatttctctccaggcacggtatacggcagatgagaacaggatgggcagatgggccagagtttgtaactcagtgcccaattcgaccaggaggaagttacacctaccggtttacagttcaaggacaggaaggcactctatggtggcatgctcatagctcatggctcagagccactgtctatggtgccctcatcatccacccaaaaccaggatcctcctatccattcactaagccaaagcgagaaacacccattcttcttggtaatgatcacttctctaatcactaacaacagatgatgataatgatgtggataataggataacaatttggatgcctatgactgagaatgatagattaacggtgagcataatgatgttcaggagaatggtgggatgcaaaccccatcgatgttgtgaggcaggcgactaggacaggagcagctccaaatgtgtctgatgcatacaccatcaatggtcaacctggtgatctttacaactgctccagcaaaggtttgcttcttttcacaaatactttgctctgctgacgagttcatgcaatatgcctagattttccctagtgggccatttcctccacacaaagacatatgatgtccgtggatgctcctagaaattataggaaaaaaaattaatccagtttttggaagacatccaaacttgtcctctcggggcaaaagagggccaccaatttaccggggcattctctgtcctttagaaaaagatgacacacctaaagaaatcagagcaattccttcaaatttttgaagcaaatccttactcttccaactcgaatccataaccagcttgacactggggactgggagagtggatttgaccatatttacccggaggttcatacattggacagaaccaaacacccaggaagcaaccaatttctactgtcctataacagctaaaacttaaatcttcatcatggatgcttagatcatgcctacaaaaaatcttctgaccttgttttattggtatgcagacactgtgatagttccaattgacagtggtgagaccaacctcctccgagtcatcaactctgggcttaaccaagagctcttcttcactgttgccaaccacaagtttacagttgtctcagctgatgcatcctataccaaacccttcaccacctcagtcatcatgctaggacctggtcaaaccactgacgtcctaatcaccggtgatcagccaccagctcgttattacatggccgcacgtgcctatcaaagtgctcaaggtgcaccatttgacaatactaccaccacagccatacttgaatacaagtctgctccttgtcctgctaagaagggtgtctccaccacccctgttttcccttctttgcctgctttcaatgacacggccactgtcacagccttcagtaagagcttcagaagtcctgctaaagtcgaagtccccacagacattgatgaaagcctcttcttcactgttggcctagggctcaataggtgcccaccaaagtttaaatccagccagtgtcaaggtcccaatggaacccgcttcaccgccagcatgaacaatgtctcttttgttctcccatccaacttttccttactgcaagctcaccagcaaggtatccctggagttttcaccactgattatcccgcagctccaccagtaaaattcgattacactggtaatgtgagccggtcactctggcaacccgttccaggtactaagttgtacaagttgaagtatgggtcaagagtccaggttgtattacagggaacaagtatcttcacagctgagaaccaccccatccatcttcatggatacgatttctacatcattgcagaaggtttcggaaacttcaaccccagtactgatacatccaagtttaatcttgttgatccacctcttaggaatacagtggccgtacctgtgaatggttgggcagtcatcagatttgttgccgataatccaggtaaaattacttgaatttcccaaaaattctacttccaatatatttcaaatttttgccctcaactttcaattcatttgtctggcaaaaacaggagtttggctaatgcactgccacttggatgttcatatcacctggggtctggccatggctttcctggtggagaatggagttggggctttgcaatcgatagagactcctccagcggatttgcctctgtgc

SEQ ID NO: 345 - DNA, 1734 bp, Ricinus communis
atgggagacatcaccaatcacatctttgccaattcttgcttcctcttctttggccttctgcttctgctagcttcaacattgtcattagccaatgcaaaagttcaccaccatgactttgttgttcaagcaacaaaagtaaagaggctgtgtaaaactcacaatactattacagtaaatgggatgtttcctggaccaaccatagaagtgaacagtggagacacactggttgtcaaagttaccaacaaagctagatacaatgttaccgttcactggcatggcattagacagatgagaacaggatgggctgatgggccagaatttataactcagtgcccaattagaccaggagggagttacacctacaggtttacaattgaaggacaagaaggaacactgtggtggcatgctcatagctcatggcttagagccactgtttatggtgctctaattatttacccaaaagatggaacctcatatccttatgctaagcccaaaagagaaacacccattcttcttggagaatggtgggatgctaaccctattgatgtggtgagggaggcaactcgaacaggagctgctccaaatatatctgatgcttataccatcaatggtcaacctggtgatctttataactgttctagtaaagaaactgtcatcgttccaattggatccggcgagacacacctcctccgagtcatcaacgctgcgctcaatcaaccactgttctttacaattgccaaccataagttcacagttgtaggtgctgatgccttgtatctcaaaccctttagcacttcagtcatcatgctgggaccaggacaaacaaccgatgttctgatctccggtgaccagccaccagcaagatactacatcgcagcacgtgcctatcaaagtgcacaaaatgctccatttgacaataccacaactactgcaattcttgaatataagtctgccccttgccctgctaagtgcctgactagcaagccaattatgcctccattgcctgctttcaatgatacacctacagtcactgcctttagcaagagcctcagaagtcccagaaaagttgatgttcccactgaaattgatgaaaatctcttctttacaattgggttaggactcaataaatgtcctaagaattttcgggctaggcgatgccaaggaccgaatggtactcgcttcacttctagcatgaacaacgtatcttttgtgcttccatccaacttttccttgctccaagctgctaggcaaaacattcctggagttttcaccactgattttccagctaagcctcctgttaaattcgattatactggtaatgtaagccaatcactttggcaacctgttccaggcactaaattgtacaaattgaagtatggatcaagggtgcagattgtgttacaggacacaagtattgtcacacctgagaaccacccaattcatcttcatgggtatgatttctatgtcattgctgaagggtttggaaacttcaatcctaaaaaagatacagctaaattcaaccttgttgatccaccaatgaggaatacagttgcagtaccttcaaatggatgggcagtcattagattcgtcgcagacaatccaggtgtatggataatgcattgtcacttggatgttcatatcacatggggactggccatggctttcttggtagaagatggaattggagagctgcagaaactagaacctcctccaaatgatctgcctttatgctag

SEQ ID NO: 346 - DNA, 1980 bp, Populus trichocarpa
ttgctcctgcttctggcctcagcaatgtcattagctattgcaaaaacccaccaccatgattttactgttcaagcaacaaaagtgaaaaggctatgcaaaacccacaacagcatcacagtaaacggaatgttcccggggccaaccttggaagtgaagaacggagacactctagttgtaaaagttgtgaacagagcccgatataatgttaccattcactggcacggtattaggcaaatgagaacgggctgggcagatgggccagaatttgtgacacaatgcccaattagaccaggtggaagttacacctacaggtttactattgaaggacaagaaggaacactttggtggcatgctcatagctcctggcttagagccactgtttatggtgctctaatcatccatccaagagaaggatcatcgtatccattctctaagccaaagcgtgaaacacccatactccttggtgaatggtgggatgcgaaccctattgatgtggtgagggaagcaactagaaccggagcggctcctaatatatctgatgcttataccattaacggtcaacctggtgatctttttaactgctccagcaaagatactaccatcgttccgatcgactctggtgagacaaacctcctccgagtcatcaatgctgcactcaatcaaccacttttcttcaccatagccaaccacaagtttacagttgtcggtgccgatgcatcctatcttaagccctttaccacctcggtcatcatgctaggacctggccaaaccacagatgttttgatctctggcgaccaactacctggccgatattacatggcagcccgagcctatcaaagtgcgcaaaatgcaccatttgacaatactaccaccactgccattcttgaatacaaatctgttctttgccctgcgaagtgtacaaagaaaccatttatgccaccacttccagcttacaatgacacagctacagtcacagccttcagtagaagctttagaagtcctcgaaaagttgaggtcccgaccgatattgatgagaacctcttcttcacaatcggcctaggactcaacaattgtcccaaaaactttagagctaggcggtgtcaaggacccaacggcactcgtttcactgctagtatgaacaatgtgtcctttgtgtttccttcaaaagcctcgctattgcaagcatataagcaaaaaattcctggagtcttcaccaccgattttccagcaaaaccacaagtaaaatttgactacactggcaatgttagtcgttccctctttcaacctgctcgtggaactaagctgtacaagttgaagtacggatcaagggtgcagattgtgctacaggacacgagcatcgtcacaccagaaaaccatccgatccatctccatggatacgacttctacatcatcgctgaaggatttgggaatttcaatcccaaaactgataaatccaaattcaaccttgttgatccaccaatgaggaatacagttgcagtacccgtgaatggatgggcggttattagatttgttgctgacaacccaggtgtgtggctaatgcactgtcacttggatgttcacatcacatggggtctggccatggcgtttttggtagaagaggggattgggattttacagagtgtcgagcctcctccagccgatctgccaatatgttaattaatatcaatacataaaagaagattttacccattgttgacactcatagaagccaacaactacaggattttctttcacctttttttctgtttcttgtagtcaatttcccatgttgttcttccctgcatttggggacatatttcattattgtattcttcctctctgggcttacaagccttagaaacaagagctgccatcttattttagttattgacctgcttgttattgattttccagagctgtttttgtttctcttcgttaataatacattcaaactttttgaaaaaaaaaaaaaaaaaaaaa

SEQ ID NO: 347 - DNA, 2044 bp, Vitis vinifera
atggaggccttgagctgctgcattgccaattctaggtctttcctcttgggccttttgcttctccttgcttctgcagtgttctttacagaagccgaaacccaccatcatgattttgttgttcaagccaccccggtgaagaggctgtgcaaaacccacaacaccataacagtgaacgggcaatacccggggccaacgttggaaataaacaatggagacaccctagaagtcaaagtcaccaacaaagctcgatacaatgttaccattcactggcacggtatacggcagatgagaacaggatgggcagatgggccagagtttgtaactcagtgcccaattcgaccaggaggaagttacacctaccggtttacagttcaaggacaggaaggcactctatggtggcatgctcatagctcatggctcagagccactgtctatggtgccctcatcatccacccaaaaccaggatcctcctatccattcactaagccaaagcgagaaacacccattcttcttggagaatggtgggatgcaaaccccatcgatgttgtgaggcaggcgactaggacaggagcagctccaaatgtgtctgatgcatacaccatcaatggtcaacctggtgatctttacaactgctccagcaaagacactgtgatagttccaattgacagtggtgagaccaacctcctccgagtcatcaactctgggcttaaccaagagctcttcttcactgttgccaaccacaagtttacagttgtctcagctgatgcatcctataccaaacccttcaccacctcagtcatcatgctaggacctggtcaaaccactgacgtcctaatcaccggtgatcagccaccagctcgttattacatggccgcacgtgcctatcaaagtgctcaaggtgcaccatttgacaatactaccaccacagccatacttgaatacaagtctgctccttgtcctgctaagaagggtgtctccaccacccctgttttcccttctttgcctgctttcaatgacacggccactgtcacagccttcagtaagagcttcagaagtcctgctaaagtcgaagtccccacagacattgatgaaagcctcttcttcactgttggcctagggctcaataggtgcccaccaaagtttaaatccagccagtgtcaaggtcccaatggaacccgcttcaccgccagcatgaacaatgtctcttttgttctcccatccaacttttccttactgcaagctcaccagcaaggtatccctggagttttcaccactgattatcccgcagctccaccagtaaaattcgattacactggtaatgtgagccggtcactctggcaacccgttccaggtactaagttgtacaagttgaagtatgggtcaagagtccaggttgtattacagggaacaagtatcttcacagctgagaaccaccccatccatcttcatggatacgatttctacatcattgcagaaggtttcggaaacttcaaccccagtactgatacatccaagtttaatcttgttgatccacctcttaggaatacagtggccgtacctgtgaatggttgggcagtcatcagatttgttgccgataatccagtttggctaatgcactgccacttggatgttcatatcacctggggtctggccatggctttcctggtggagaatggagttggggctttgcaatcgatagagactcctccagcggatttgcctctgtgctaagatcatcaacataaaaaccacccacagttgaacttcatatatatgccttataaaaggggcacctatttttctactcttagcatacggattattcagtgttgttttccccgcatttggggccttagttcttgctttctatttcattgttctttttgatagataaagcgggtccagatggtgctgtgttgcatcacagagcgccatcagatttaaaatcagaccccatttgtttccttgttttttttcttttttcggttgtattgtttcaagaaacatcatcagagataaattgaagttcagaaagttattatta

SEQ ID NO: 348 - DNA, 2018 bp, Populus trichocarpa
atggaggttatcaaccgtatctttgctaatcgccattgttctttcttcttgctcctgcttctggcctcagcaatgtcattagctattgcaaaaacccaccaccatgattttactgttcaagcaacaaaagtgaaaaggctatgcaaaacccacaacagcatcacagtaaacggaatgttcccggggccaaccttggaagtgaagaacggagacactctagttgtaaaagttgtgaacagagcccgatataatgttaccattcactggcacggtattaggcaaatgagaacgggctgggcagatgggccagaatttgtgacacaatgcccaattagaccaggtggaagttacacctacaggtttactattgaaggacaagaaggaacactttggtggcatgctcatagctcctggcttagagccactgtttatggtgctctaatcatccatccaagagaaggatcatcgtatccattctctaagccaaagcgtgaaacacccatactccttggtgaatggtgggatacgaaccctattgatgtggtgagggaagcaactagaaccggagcggctcctaatatatctgatgcttataccattaacggtcaacctggtgatctttttaactgctccagcaaagatactaccatcgttccgatcgactctggtgagacaaacctcctccgagtcatcaatgctgcactcaatcaaccacttttcttcaccatagccaaccacaagtttacagttgtcggtgccgatgcatcctatcttaagccctttaccacctcggtcatcatgctaggacctggccaaaccacagatgttttgatctctggcgaccaactacctggccgatattacatggcagcccgagcctatcaaagtgcgcaaaatgcaccatttgacaatactaccaccactgccattcttgaatacaaatctgttctttgccctgcgaagtgtacaaagaaaccatttatgccaccacttccagcttacaatgacacagctacagtcacagccttcagtagaagctttagaagtcctcgaaaagttgaggtcccgaccgatattgatgagaacctcttcttcacaatcggcctaggactcaacaattgtcccaaaaactttagagctaggcggtgtcaaggacccaacggcactcgtttcactgctagtatgaacaatgtgtcctttgtgtttccttcaaaagcctcgctattgcaagcatataagcaaaaaattcctggagtcttcaccaccgattttccagcaaaaccacaagtaaaatttgactacactggcaatgttagtcgttccctctttcaacctgctcgtggaactaagctgtacaagttgaagtacggatcaagggtgcagattgtgctacaggacacgagcatcgtcacaccagaaaaccatccgatccatctccatggatacgacttctacatcatcgctgaaggatttgggaatttcaatcccaaaactgataaatccaaattcaaccttgttgatccaccaatgaggaatacagttgcagtacccgtgaatggatgggcggttattagatttgttgctgacaacccaggtgtgtggctaatgcactgtcacttggatgttcacatcacatggggtctggccatggcgtttttggtagaagaggggattgggattttacagagtgtcgagcctcctccagccgatctgccaatatgttaattaatatcaatacataaaagaagattttacccattgttgacactcatagaagccaacaactacaggattttctttcacctttttttctgtttcttgtagtcaatttcccatgttgttcttccctgcatttggggacatatttcattattgtattcttcctctctgggcttacaagccttagaaacaagagctgccatcttattttagttattgacctgcttgttattgattttccagagctgtttttgtttctcttcgttaataatacattcaaactttttgaattctctgct

SEQ ID NO: 349 - DNA, 1746 bp, Ricinus communis
atggagtccctaacccacatttttgctaatcatcttttagcttccttcttaggccttctattagtcattgcttctgcattgtcctcagccaatgcaacaccaatgactcacaaccatgagtttgttattcaagcaacgtcagtgaagaggctgtgcaagactcaaaatgtcatcactgttaatgggatgtttcctggaccaacactagaagtgaacaatggagacactctagttgttactgttaccaacagagctcagtacaacgttaccatccactggcatggtatcaggcaaatgaggactggatgggcagatggaccagaatttgtaacacagtgtccaattaggccaggagggacatacacatacaggtttactattcaagcacaagaaggaacactttggtggcacgctcatagctcatggctgagagccactgtttatggagctttaatcattcatccaaaagaaggatcttcatatccattccctaagccaaagcgtgaaacaccaattattcttggtgaatggtggaatgcaaatcccattgatgttctgaggaaagcaacaagaacaggaggggcaccaaatgtttctgatgcgtacaccattaatggtcaacctggtgatctttacaactgttctagccaagatactgtgattgttccaatagattccggcgagacaaaccttctccgagtcatcaatgctgcaatgaaccaaccacttttctttactgtagctaaccacaggctcacagttgttggtgctgatgcttcctacacaaaacccttcaccaccagtgtcctaatgctaggacctggacagaccacagatgttttgatctccggcgaccagaaacctgcccggtactacatggcagcacgcgcataccaaagcgcccaaaatgcccagtttgacaacactactactactgccattcttgagtacaaatctgcaccttgtgctgccaaaaactgctcatcaaacaaaccaattatgccaccattaccagcatataatgacacagcaactgtcactgcttttagtaccagcttcaggagtcgtaacaaagtcttagtcccaacagaagttgatgaaaatctattctttacagttggtttaggactcaacacttgccccccaaattttaacaagtctagccaatgtcaagggcctaatggaacaagattcgcagcaagcatgaacaatgtgtcatttcaactcccatcaaatttttccatcttacaagctcatcagttaggaattccaagagtatttacaactgatttccctgccagcccaccattaaaattcgactacacaggtaatgtcagccgatcattatggcaagctgttgctggtaccaaggtttataaactgaagtacggatcaagagtgcagattgtgctacaagatacaagtattgtcacatctgaaaaccacccaattcaccttcacggatatgatttctatatcattgctgaaggctttggaaactttaatccacagactgatacttctaaatttaaccttgttgatccacctctgagaaacacagttggagtacccgttaatggatgggcagtcattagattcgtcgcggataacccaggtgtctggttaatgcactgtcacttggatgttcatattacatggggcttggccatggctttcttggtagagaatggagttggtgtattacaatctatagaagctccaccagaagatctgcctccatgctaa

SEQ ID NO: 350 - DNA, 1786 bp, Arabidopsis lyrata
aatttatcgaaatcgcattgatggagatcgtcaagagcctcatcttcatctctttggctgtcgttcttctcttcgcttccatagcagaagccaacattaaagcacaccaccacgagttcattatacaagcgacgaaagtgaagagactatgtgaaacacacaacagcattacggtcaacggaatgtttcccggtccaatgcttgtagtcaacaacggtgatactctcgtcgtcaaagttattaaccgggctcggtacaacataacaatccactggcacggtgtgaggcaaatgcgaaccggttgggcagatggaccggaatttgtgacccaatgtccaatcagaccgggatcaagctacacgtaccggtttactatccaaggacaagaaggtacactttggtggcatgctcatagttcgtggcttagagccactgtctacggttcgcttcttgtccttcctccggctggctcgtcttacccattcacaaatccccaccgcaacgtccctctccttcttggtgaatggtgggacgcaaatccggttgatgtgttgagagaatcaatacgaaccggaggagctcctaataactccgacgcttacaccatcaatggtcaaccaggcgatctctataaatgctcttctcaagatacgacaatagtaccaataaacgttggtgagactatactactacgggtaataaactcagcattgaaccaaccgttattcttcacggttgctaaccacaagctcacggtggtgggagctgacgcctcttacctaaaacccttcaccactaacgtgatagttcttggcccgggccaaaccactgatgtcctcataaccggtgaccaaccaccaaaccgctactacatggccgcaagagcttaccaaagcgcccaaaacgcaccatttggaaacacaactacaaccgcaatccttcaatacaaatccgccccttgttgcggtgtaggcggcggaagtggaaccaagaagggtatttccgtcaaactaatcatgcctatcctccctgcctacaacgacaccaacaccgtcacacgtttcagccaaagcttccggtcactaagaagagctgaggttccgacagaaatcgacgagaatctctttgtaactgtcggacttggtctcaacaactgtcctaagaactttagatcaagaagatgtcaaggtcctaacggcacacgtttcactgcatcgatgaacaacatttcatttgctcttccaagtaactactcactccttcaagctcaccaccatggaatccccggagtcttcacaaccgattttccggccaagcctccggttaaatttgattacacaggtaacaacataagccgatctctctaccaacctgatagagggactaagctatacaaactcaagtatggatcaagggttcagattgttcttcaggacactggtatagtaactcctgaaaatcatccaattcatctacacggctacgatttctacattatcgccgaaggtttcggtaacttcaatcccaagaaagataccgcgaaattcaatcttgaagacccgcctctcagaaatactgttggtgtacctgttaatggttgggccgtcatcagattcgtcgcagacaaccctggggtttggataatgcactgtcatctagatgcacacatatcttgggggctagccatggctttcttggttgagaatggaaatggagttttgcagacaatggagcagcctcctgctgatttacccgtatgttattaggagttcaagatcctttgtaa

SEQ ID NO: 351 - DNA, 2743 bp, Vitis vinifera
atggaggccttgagctgctgcattgccaattctaggtctttcctcttgggccttttgcttctccttgcttctgcagtgttctttacagaagccgaaacccaccatcatgattttgttgtatgtcttctcttctctcttccttccttttacttgcttctttcctctcttggccttggattctgactatctgcattgatgtaacaggttcaagccaccccygtgaagaggctgtgcaaaacccacaacaccataacagtgaacgggcaatacccggggccaacgttggaaytaaacaatggagacaccctagaagtcaaagtcaccaacaaagctcgatacaatgttaccattcactggtaattccatgcatgcatacatgtttcctctgtacaaaaatgaattgawmtgaaggtatctggatagggcatgaatgcatgcaggcatgcaggcatgctcctacaaaagaaccaaccaakgtatatatttcwtccaatgaacytactaatgtatatasaaagactcttcktaccttctatacttatcttyctattatttctctccaggcacggtatacggcagatgagaacaggatgggcagatgggccagagtttgtaactcagtgcccaattcgaccaggaggaagttacacctaccggtttacayttcaaggacaggaaggcactctatggtggcatgctcatagctcatggctcagagccactgtctatggtgccctcatcatccacccaaaaccaggatcctcctatccattcactaagccaaagcgagaaacacccattcttcttggtaatgatcacttctctaatcactaacaacagatgatgataatgatgtggataataggaraacaatttggatgcctatgartgagaatgatagattaacggtgagcataatgatgttcaggygaatggtgggatgcaaacccratcgatgttgtgaggcaggcgactaggacaggagcagctccaaatgtgtctgatgcatacaccatcaatggtcaacctggtgatctttacaactgctccagcaaaggtttgcttcttttcacaaatactttgctctgctgacgagttcatgcaatatgcctagattttccccagtwgtgggccatttcctccacycaaagacatatgatgtccgtggatgctcctagaaattamaggaaaaaaaattaatcgagtttttggaagacatccaaacttgtcctctcgyggcaaaagagggccaccaatttacrggggcattctctgtcctttagaaaaagatgacacacctaaagaaatcagagcgattccttcaaatttttgaagcaaatccttactcttccaactcgaatctataaccagcttgacactgtggggactgggagagtggatttgaccatatttacccggaggttcatacattggacagaaccaaacacccaggaagcaaccaatttctactgtcctataacagctaaaatttaaatcttcatcatggatgcttagatcatgcctacaaaaaatcttctgaccttgttttattggtatgcagacactgtgatagttccaattgacagtggtgagaccaacctcctccgagtcatcaactctgggcttaaccaagagctcttcttcactgttgccaaccacaagtttacagttgtctcagctgatgcatcctataccaaacccttcaccacctcagtcatcatgctaggacctggtcaaaccactgacgtcctaatcaccggtgatcagccaccagctcgttattacatggccgcacgtgcctatcaaagtgctcaaggtgcaccatttgacaatactaccaccacagccatacttgaatacaagtctgctccttgtcctgctaagaagggtgtctccaccacccctgttttcccttctttgcctgctttcaatgacacggccactgtcacagccttcagtaagagcttcagaagtcctgctaaagtcgaagtccccacagacattgatgaaagcctcttcttcactgttggcctagggctcaataggtgcccaccaaagtttaaatccagccagtgtcaaggtcccaatggaacccgcttcaccgccagcatgaacaatgtctcttttgttctcccatccaacttttccttactgcaagctcaccagcaaggtatccctggagttttcaccactgattatcccgcagctccaccagtaaaattcgattacactggtaatgtgagccggtcactctggcaacccgttccaggtactaagttgtacaagttgaagtatgggttcaagagttccaggttgtattacagggaacaagtatcttcacagctgagaaccaccccatccatcttcatggatacgatttctacatcattgcagaaggtttcggaaacttcaaccccagtactgatacatccaagtttaatcttgttgatccacctcttaggaatacagtggccgtacctgtgaatggttgggcagtcatcagatttgttgccgataatccaggtaaaattacttgaatttcccaaaatttctacttccaatatatttcaaaattttgccctcaactttcaattcatttgtctggcaaaaacaggagtttggctaatgcactgccacttggatgttcatatcacctggggtctggccatggctttcctggtggagaatggagttggggctttgcaatcgatagagwctcctccagcggatttgcctctgtgct

SEQ ID NO: 352 - DNA, 542 bp, Glycine max
ctcaaggagatggctctgggaagaggcagtgcagtggttctactactttgcttcttgctgcttcactctcagatggctcgtgctgccacctacacagttggagattctgggggttggacctttaacactgttgcctggcccaaaggaaagctctttcgggctggtgacacacttgctttcaattatagccctgggactcacaatgtggtggccgtgaacaaggctggatatgatagctgcaagactccaagaggagccaaagtgtataagtcagggacggatcagatcagacttgccaagggacagaactacttcatctgcaattatgttggtcactgcgagtctgggatgaaaattgccatcaacgctgcctgagtttaataatatggttaactacccatacatattgtattgtaatgcaaattgcaccctttagtgggaagttagctcctttaaataatgttaagaaaaaaagtagctatgtgtgctcttctataaatgtcacttatctataaagaataaatgtcggcagtcggtgatcc

SEQ ID NO: 353 - DNA, 730 bp, Medicago truncatula
ttttttttttttttttttttgaatcaattgtagttcccttcaaacaattggttcgctacataacatcctgagagacccagaaatcatattacacaaattatttgttactactagatccatgatgactaacatatagcagcatacatggaacacttaacacttcacacccacctatccacttatttaatttaaggataataaaacttctacatgattcaagaagtacataaagaacaggtacaacaagaggtatgaaaattgaaaaacacacacatatataagttgaaccctaaattattaggtatattagactttaatatagcaaaatttaagcagcattgatggcaattttcattccggactcgcagtgacccacaaagttgcaaatgaagtagttttgtcccctagcaagcctgatctgatcctttcctgacctatacacttttgctcctcttggagtcttgcaactatcataccctcctttgttcaccgccaccacattgtgagctgatggactatagttgaacacaagggtatcaccagccctaaagcgttttccattaggccatccaacagtgttaaaggtccaacctccaggacctccaacagtgtaggtagcagcatgagccaactcagagttaagaacaaaaaagcagactagtagaaccaatgcactggctcttcccagagccatgtttctctactattatttgctagaaaactgtgaaag

SEQ ID NO: 354 - DNA, 625 bp, Glycine max
ggactcacctccaaaacatatctccttctttacttagttcaactcaacccaaggagataattaatatggctctgggaagaggcagtgcagtggttctactactttgcttcttggtgcttcagtctgagatggctcgtgctgccacttatagagttggagattctaggggttggacctttaacaccgttacctggccccaaggaaaacgctttagggcaggtgacacacttgcgttcaattatagtcctggggctcacaatgtggtggcggtgagcaaggctgggtatgatagctgcaagacaccaagaggagccaaagtgtatcggtcagggaaggatcagatcagacttgctaggggacaaaactacttcatctgcaattacgttggtcactgcgagtctgggatgaaaattgccatcaacgctgcatgactttcttctctactcatacgcatctattctaacgcaaactgcacccctttagtgggaatcaacttgtctcctttaaataaatggatagatataggacgtgtgtgttatttaagaaaaagggtagctatgtgtgctctcctatgtatgtaacttattatctattgatgattcagaataaatgtcggcagtatatc

SEQ ID NO: 355 - DNA, 708 bp, Cicer arietinum
agctcattattccaaattcaattacatccatatcccaaaatttccttttacaaatttctagcaattacatccatatggctttgggaagaggcagtgcattggttgttctactagtttgctttttggtgattcactctgagttggctcaggctgccatctacactgttggaggtgctggtggttggacctttaacactattgcctggcctaatgggaaaaactttaaagctggtgatacacttgtattcaactatagtccgggtgcacacaatgtggtggcagtgagcaaagcagggtatggtagctgcaagactccaagaggagccaaagtgtatcggtcaggaaaagatcagataaggcttgctagaggacaaaactatttcatctgcaattatgttggtcactgcgagtctggaatgaaaattgccatcaatgctgtttgatttttattattataataataatattgtgtgcttttatctatgtgctttcatcatacctcttgtacctgttctaatttatacacttcgtgaatcatgtagaagttttcaataaatggaaaggtgggtgtgaattgtggtgtgtaccatttatgttagtaaatgttattcatcatgggtgtagtaagttgtaacaatacttttgtgtaatacggcccaattgttttgagagaacggcatatgacttgaaaaaaaaaaaaaaaaaaaa

SEQ ID NO: 356 - DNA, 635 bp, Populus trichocarpa
aagcaaaatccctcaactagctagccagcaaagatctgtcctctttgtctcttatctctcaaaaaatggttcagggaagaggcagtgcgatggtggcgacagtcgcggttatgctgtgcatgctgctgctccattttgatatggctcacgcagcaacctacactgttggaggccctggtggctggaccttcaatgtttctggctggcctaaaggaaagagttttaaagctggtgatatacttgtattcaattacagcactgcagcccacaatgttgttgctgtgaacaaggctggttacagttcatgcacgagccctagaggtgccaaggtttacacatcaggaaaggatcagatcaagctcgtgaagggacaaaatttcttcatctgtagctttgctggacactgtcagtctggaatgaaaatagctgttaatgctgcgtgaaatggtggtgttgggggatggataatcagagatgagtcatgagattcatgatcttttccttttatgtcttgatatatatatatatatgcaactctatgtgctagcgtgttgatcagtggctagctaccccttgtactagttaattattactaacatataaaaggaataaacaagaaataatgttgcttgctt

SEQ ID NO: 357 - DNA, 803 bp, Glycine max
ggaacacaaccaaaattcgctacttgttaagggctgcactataccagaatgtctcagggaagaggcagtgcatctttgcctattgtggtcactgtggtttcactactgtgccttttggaacgtgctaacgcagcaacttactccgttggaggacctgggggatggaccttcaacactaatgcttggcccaatggaaaaagattcagagctggtgatatcctaatcttcaactatgactcaacgacccacaatgtggttgctgtggacagaagtggatacaacagctgcaagacaccagggggtgctaaagtgttcagttcagggaaggaccaaatcaaactagcaagagggcagaactacttcatatgtaactaccctggtcactgcgaatctgggatgaaagttgccattaatgcgctgtgatatactagtagtgctcaatacttcttgaaatgaagtaactgttaagttaatggtcaattaactagcattagtaaatgttagtgtgtgatattgtatgtagtcttttttttatatgaagctgttatatatattaattactatagtttgtgaagcaagtgggagtgagctggcttggtagatcaattgttcctgtatttggtatgtgttgtgtggtgggagtatgataatggtccattagtcacgggctcttcttattagacattggatctgcatattttgaaattattcaagtctgagccaattatctgtatctagtgtctcggtgtcacagtgtgtggttgtattactgaatttgttgccttcataagttaaatttctaa

SEQ ID NO: 358 - DNA, 381 bp, Ricinus communis
atggctcagggaaggggcagtgcaaatctagccatagccactgtggttgcactactgtgcctgctgactctcactaagcaagttcgtgctgcaacttacactgttggtggctctggcggttggactttcaatgtagacagttggcctaaaggcaaacgttttaaagctggggatacactcgtattcaattatgattcaacagtgcacaatgtggtagctgtgaacaaaggaagttatacaagctgcagtgctccagcaggtgcaaaagtgtacacatcagggcgagatcagatcaagctggcgaagggacaaaacttcttcatatgcggtatcagtggccactgtcaatctggcatgaaaattgctattactgctgcatga

SEQ ID NO: 359 - DNA, 803 bp, Glycine max; identical to SEQ ID NO: 357

SEQ ID NO: 360 - DNA, 733 bp, Medicago truncatula
gactcaaccatctcactataattgctataggcatctatagtttataccaagaaaaatagtgaatatgactgagggaagaggcagtgcttctatgaacatggtcactctaatttcactgctgtgccttttggttctggctgaaagtgctaatgcagcatcttacaccgttggaggaactgggggatggacatacaatactgatacttggcctaatggaaaaaagtttaaagctggtgatgtgctcagcttcaactatgattcaaccacacacaatgtggttgccgtggacaaaagtggatacaacaactgtaagacaccgggaggtgctaaagtgttcagttcagggagtgaccaaattaggctatcaagaggacaaaactacttcatatgcagctatcctggtcactgccaatctgggatgaaggtttccatctatgcagtttagtcctctagctaagtgaccacagtagttaattagttaattaatggtcaattagctgtagtggtaatggtatgatttctttaattaccatataaataacagcatgagtactaggctactagctatatgttgatttggtgatcaattactattattattattatgtatgtaaaaggagtgtggtgaggtggctttctttataaaattgtatgtttgatatttttatttttgtggtgtgctgtgtgatgtgaggatatatatatatatgtatgatagagattaactatcttg

SEQ ID NO: 361 - DNA, 642 bp, Glycine max
ggagggtttacactgaggaggcacaccttcaggagcagtcatggttctcaaaaccgagctgtgccgattcagcggtgccaagatctacccagggaaaggcatcagatttgttcgtggtgattctcaggttttcctgtttgctaactcaaaatgtaagaggtatttccacaacaggttgaagccgtcaaagctcacgtggactgcaatgtaccgaaagcagcataagaaggatattgctcaagaagctgtgaagaagagaagacgtgctaccaaaaaaccttactctaggtctattgttggtgctactttagaagttatccagaaaaagagaaccgagaagccagaagttcgagatgcagctagggaagcacagcttcgtgaaattaaggagaggatcaagaaaactatggatgacaagaaagctaagaaagcagaagttgcagctaagtcccaaaaatcacaagggaaaggaagtatttcgaagggtgccatgcccaaaggtcccaaacttggtgggggaggtgggaaacgctgagcttttaagttttgttcttatttttggctacaatttaaatagagctgttttgaaacttctgtactgatattttatttgcatgattaatatgttttattaagaaaccc

SEQ ID NO: 362 - DNA, 746 bp, Populus trichocarpa
ccaaaccctagtctggtcttccttcttccctgccaaaaaatcagtagctgctgccgccatggttctcaagactgaactctgccgcttcagtggggcgaagatctatcctggcaagggtatcagatttattcgctcagattcccaggtcttcctctttgccaattctaaatgcaagaggtacttccacaacaggctgaagccctcaaagctaacctggacagctatgtacaggaagcagcataaaaaggacattgctgcagaaactattaagaagaggcgtcgcgccacgaaaaaaccttactcaaggtccattgtaggggctactttggaggtcatacagaagaagcgaactgagaaacctgaagttcgtgatgctgcaagggaggctgcactccgtgaaattaaggagaggattaagaaaacaaaggatgagaagagagccaagaaggctgaggtaacagccaaggtacaaaagagcagcaaaggtagcgtgccaaagggtgctgcaccaaagggccccaagcttggcgggggtggaggaaagcggtgaaatccttccttctttgttgtcctattaactcgttaaagaattttgtattggatattttacatggatcaaggtctgttgttgccattttgttctttaagagttaacctactcttttctctttcagcgttaattatccatggtttgaaagttgttgaattttattattatggattgcacgcactgccaatgctgggga

SEQ ID NO: 363 - DNA, 690 bp, Prunus avium
aacaagaagagcagcagtcgataacagcaggagcaggaggcagcagccatggttctcaagaccgaactctgtcgttttagcggggccaagatttacccaggaaagggcatcagatttattcgttctgactctcaggtcttcctgtttgccaactcaaaatgcaaaaggtacttccacaacaggttgaagccatcaaagcttacctggacagccatgtacaggaagcagcacaaaaaggatattgctcaagaagctgtgaagaagaggagacgtaccaccaagaagccttactcaaggtctattgtgggtgccacactcgaggttatccagaagaggagaactgagaagcccgaggttcgtgatgctgcaagggaagctgctctccgtgaaatcaaggagaggatcaagaaaaccaaggatgaaaagaaggccaagaaggcagaagttacaaaatcccaaaaatctcaaggcaagggtagcattgccaagggaggtgcacaaccaaagggaccaaagcttggggggtggcggtggcaagcgctgagccactgttctgttgcctacttgttgggaagtagagtaatagataggacgtgtttgtttgtttgaagattttgtaaggattgattggccatgcttgatggtccattcgtatttttcttttatctatcttatatctgtcactttgat

SEQ ID NO: 364 - DNA, 659 bp, Glycine max
agggtttacaccgagcagccacacctcccgcagcagccatggttctcaaaactgaactatgccgattcagtggtgccaagatctacccaggaaagggcatcagatttgttcgtggtgattctcaggttttcctgtttgcaaactcaaaatgtaagaggtatttccacaatcgcctgaagccttcaaagctcacctggactgcaatgtacagaaagcaacacaaaaaggacattgctcaagaagctgtgaggaagaggagacgtgctgccaaaaagccttactctaggtccattgttggtgcgaccttggaagtaatccagaaaaagagagctgagaagccagaagttcgagatgcagctagggaagctgctcttcgtgaaattaaggagaggatcaagaaaacaaaggatgagaagaaggctaagaaagcagaagttgcttccaagtcgcaaaaagcaggagggaaaggcaatgtttctaagggtgctatgcccaaaggtcccaaacttggcggtggaggcgggaaacgctgagcttctagttttgttcctaattttggctacaatttaaatagttgttttgacactgctgtactgatatttcatttggaagattaatcttttattttactgttttactaaacctgtgtttggatattc

SEQ ID NO: 365 - DNA, 697 bp, Cicer arietinum
gcgcattctcccctaacgcagcagccatggttctcaagactgaactttgccgattcagtggtgcaaagatctacccaggaagaggaatcagatttattcgtggtgattctcaggttttcctgtttgttaactcaaaatgcaaaaggtatttccacaaccgtttgaagccttcgaagcttacctggactgccatgttcaggaagcaacataaaaaggacgctgctcaagaagctgtgaagaagaggcgtcgtgctaccaaaaagccatactctaggtccattgttggtgctactttggaagtcattcagaagaaaagaactgagaagcctgaagttcgtgatgccgctagggaagctgctcttcgtgaaattaaggagagaatcaagaaaactaaggatgagaagaaagccaagaaagcagaagtagcatctaaggcacaaaaatctcaaggcaaaggaaatgttcagaagggtgctttgcccaaaggtcctaaaatgggtggcggcggtgggaaagcctgagcgttccatttttggttacatttaaatatatttcttttgaaacttctgtatccgtatatttgatttgcttgattagtctttttttgttacgttttatcaaacttgtttttggagcttccaagagaaaccagtgatcgattttgctagttatgttattttttggtgcaaaaaaaaaa

SEQ ID NO: 366 - DNA, 864 bp, Vitis vinifera
tactcgaggggtgtgtggttagggtttgggcttccttttctagggtttccgaggcgcagcagcagcaacagcagccatggttctcaaaactgaactctgccgattcagtggtgccaagatataccctgggaaaggaatcagatttgttcgttcagattctcaggtgtttctatttgccaattcaaaatgcaagaggtacttccacaaccggctgaagccatcaaagcttacctggacagccatgtacaggaagcaacataaaaaggatattgctcaagaggctgtaaagaagaggcgtcgtgccaccaaaaagccctactctaggtccattgtgggtgctacattggaggttatccagaagagaagaaccgagaaagcagaagtcagagatgctgccagggaggctgctcttcgtgaaattaaggaaagaatcaagaagaccaaggatgaaaagaaggcaaagaaggcagaagtgatggctaaggtgcagaagacacagggcaagggtaacgttcccaagggtgctgctgctccaaagggccctaaaattggaggtggtggtggtaagcgctgatttggggcttgtttggtgcggtgagagtgagatcggtaggttggaagaagttttaatttaaagttttgttgtaaggattgcttggcctggttagccagattgttattcgcatccccttttctataattagcactgtcgactgaagttgagcacgattatatagctaaatggatttttattttgtttcctatatacctgttttaatttataaaaatgagatatcaatgaagttttgtgaaatggattcatgggattcagtgatatgatcatgagttgtcattgagaaatag

SEQ ID NO: 367 - DNA, 663 bp, Glycine max
agggtttacaccgagcagcgacacctccctcccgcagtagccatggttctcaaaactgaactatgccgactcagtggtgccaagatctaccccggaaagggcatcagatttgttcgtggtgattctcaggttttcctgtttgccaactcaaaatgtaagcggtatttccacaaccgcctgaagccctcaaagctcacctggactgctatgtacagaaagcaacacaaaaaggacattgctcaagaagctgtgaagaagaggagacgcgctgccaaaaagccttactctaggtccattgttggtgccactctggaagttatccagaaaaagagagctgagaagccagaagttcgagatgcagctagggaagctgctcttcgtgaaattaaggagaggatcaagaaaacaaaggatgagaagaaggctaagaaagcagaagttacagccaagtcacaaaaagcaggagggaaaggcatttctaagggtgctatgcccaaaggtcccaaactcggtggtggaggcgggaaacgctgagcttttagttttgttcctattttttgttacagtttaaatagagttgttttgacactgatgtaccgatatttcatttgcaagattaatcttttattgtactgttttattcagcctgtgtttggatcttca

SEQ ID NO: 368 - DNA, 1169 bp, Medicago truncatula
gtgtgttaggaccgaattgtgccgattcagtggtgccaagatctaccctggtagggggatcagatttatccgtagtgattctcaggtaatataaaattggcagagattgaagtttgttattgggttttgaatttgatgtttgctataatttttattgtgattggttgcttatgagattgtgtttgttgatttttccaggttttcctgtttgtcaactcgaaatgtaaaaggtatttccacaacaagttgaagccctcaaagcttacatggactgccatgtataggaagcaacacaagaaggtgaacagttttacttaaaatcacttttgcataattgtatgtcgtagaagctatatattctgttgcaaatctgttgtatttaaggcgcgtttggattgacttatgtttgtctcataaactaccttgacaagcttatgagtatacatagaagcttatttatttgcataagctaaaaaataagccaatccaatccggcccttgctacttaaaccaaaattgccaactaatgaacttcactgtgaggggaattatttttacacctttaagctgtgatttatccttctgatattacggtacaacgtactattgcatatataatacttggaagcaacataagaagatgaatattaatatttgagattactttgcatagttgtatgtgacaaattatcatcaattgctaatcaattgtacttgtagcttgtgtaacctgattatcaaaggaacttttattatttgacaaacttttgttgtactcgttacaagttttaatatctttaagctgtaaatttatacttctgatttacctcaggattatgacgaaaggtataccacacacatctaaacatgttactatgctacttgcctttatgttaatgtttggtgtaatttgaagtcttgttgtaatttaaagttgtagtgtctgtgattaagccgtatgtaattaagcctctgttttgattgctgcatgcttatttttgtgttcctgtgattgacaggatattgcccaagaagctgtgaaaaagaggcgtagagctaccaaaaagccttactcaaggtccattgttggtgctactttggaagttattcaaaagaagagatcagaaaagccagaggttcgggatgctgccagagaagctgcccttcgg

SEQ ID NO: 369 - DNA, 575 bp, Camellia sinensis
gagggtttaaggaaatagtagctgctgccgctcgctagccatggttctcaagactgaactctgccgattcagtggggccaagatataccctggcaagggtatcagatttattcgttcagactctcaggtcttcctcttttccaattcaaaatgcaagaggtacttccacaaccggctgaagccctcaaagctcacctggacagccatgtacaggaagcagcataagaaggatgccgctcaagaagctgttaagaagaggcgtcgcactaccaagaaaccttactcgaggtccattgttggtgctactttggaggtcatacagaagagaagaaccgagaaacctgaagttcgtgatgctgccagggaggcggccctgcgtgaaatcaaggagaggattaagaaaaccaaggatgaaaagaaggctaagaaagccgaggtgactgcgaaaacacagaagtcacaaggcaaaggtatttcaaagggtgctgtaccaaagggccccaagcttggaggtggtggtggaaagcgttgcaaggccattttcggctgttgtgttttttattggggtatatgtc

SEQ ID NO: 370 - DNA, 712 bp, Medicago truncatula
ggggtttacatttacaaagagcagccaccacagcgacgaagcagccatggttctcaaaactgaactctgtcgattcagtggcgcaaagatttacccaggaagagggatcagatttattcgtagtgattctcaggttttcctctttgtgaactcaaaatgtaagaggtatttccacaacaagttgaagccttcaaagctcatatggactgccatgtacagaaagcagcacaagaaggacattgctcaagaagcggtgaagaagaggcgccgtgccaccaagaagccatactccaggtccattgttggtgctacacttgaagtcattcaaaaaaaaagaaccgagaagcccgaggttcgagatgctgctagggaagctgctcttcgtgaaatcaaggagaggataaagaaaaccaaggatgagaaaaaagccaagaaagccgaagtagcatctaaggcacaaaagtcagggaaaggcaatgttcagaagggtgctatgcccaagggtcctaaaatgggcggtggtggtgggaaacgttaagtttttccgtttttggttaccatttaaatataatttgttttcaaacttctgtactgatatattctatttgcttgattagccttttatgttactgttttatcaaacttatgtttggagcttcagagaactcggatattaaatggattttgctagttatgctatgtttatttgcac

SEQ ID NO: 371 - DNA, 21 bp, artificial sequence
  synthetic construct
tggagaagcagggcacgtgca

SEQ ID NO: 372 - DNA, 21 bp, artificial sequence
  synthetic construct
tcgcttggtgcaggtcgggaa

SEQ ID NO: 373 - DNA, 24 bp, artificial sequence
  synthetic construct
ggcttattgagtgcagcgttgatg

SEQ ID NO: 374 - DNA, 21 bp, artificial sequence
  synthetic construct
ctgcactgactcttccctggc

SEQ ID NO: 375 - DNA, 21 bp, artificial sequence
  synthetic construct
tggaggtgtcgttgccaagga

SEQ ID NO: 376 - protein, 450 aa, Glycine max; identical to SEQ ID NO: 287

SEQ ID NO: 377 - DNA, 20 bp, artificial sequence
  synthetic construct
tttagcgatgaacttcactc

SEQ ID NO: 378 - protein, 589 aa, Glycine max; identical to SEQ ID NO: 288

SEQ ID NO: 379 - protein, 124 aa, Glycine max; identical to SEQ ID NO: 303

SEQ ID NO: 380 - DNA, 1487 bp, Glycine max; identical to SEQ ID NO: 341

SEQ ID NO: 381 - DNA, 1770 bp, Glycine max; identical to SEQ ID NO: 342

SEQ ID NO: 382 - DNA, 803 bp, Glycine max; identical to SEQ ID NO: 357

SEQ ID NO: 383 - DNA, 1350 bp, Glycine max
atggttacttgtcctggttccataataatccatttcttcctcttctcagcgcctctcctttcagtcttatggtcttgttcatcagtttctgcattgaaaccccgtgccttcatcctccccatcgagaaagacccaaccacccttcagtactcaacttccatcgacatgggtacccctccactcacactagatctagtcatcgacatcagagaacgcttcctatggttcgagtgcggcaacgactacaactcctcaacctactaccctgtccgatgtgggactaagaaatgcaagaaagccaagggcacggcttgcattacatgcaccaaccaccctcttaaaacaggatgcaccaacaacacgtgtggtgtagatccattcaaccccttcggcgagttcttcgtgagtggagacgtgggtgaagacatcttgtcctcgctgcactcaacaagcggtgcacgagcaccttccactttgcacgtgccacgcttcgtctctacatgtgtgtacccagataaatttggggttgagggctttctccagggcctggctaagggcaagaaaggagttttaggccttgcaaggactgctatttccttaccaacacaactcgcagccaaatacaaccttgaacctaagtttgcactttgtttaccttcaacttcaaagtataataagcttggtgatctctttgttggtggtgggccttactatttgccacctcatgatgcttccaaatttctttcctacactccaattctcaccaacccccaaagcacaggtccaatctttgatgctgatccttccagcgagtactttattgacgtgaagtcaatcaagcttgacggtaaaattgttaacgttaacacctccctgctttctattgacagacagggaaatgggggctgcaaacttagtaccgtagttccttacaccaaattccacacttcaatataccagccacttgtgaatgactttgtaaagcaagcagcgcttaggaaaataaagagagtgacttcggtggcaccatttggggcgtgctttgattcaagaaccattggcaagaccgttactggacccaatgtgccgacaattgatctggttctcaaggggggagttcaatggagaatctatggtgccaattcaatggtcaaggtttctaagaatgtgctgtgccttggatttgtggatggaggtttggagccaggaagtcccattgcaacttcgattgtgattggtgggtatcagatggaggacaatcttttggagtttgatcttgtttcctcaaaacttggttttagctcctcccttttactccacatggctagctgttcccacttcagacttgtt

SEQ ID NO: 384 - DNA, 1767 bp, Glycine max
atggagcctgccaaaaccattcacaacaatgtcaaatactcccccatcttcttagccatctttgttctgatcttagcttcagcattgtcttcagcaaatgccaaaattcacgagcacgagtttgttgttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggcacggtgttaggcaaatgagaacagggtgggcagatggaccagaatttgtgacttcagtgcccgattgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcgcgagacacccattcttcttggggaatggtgggacgcaaaccctattgatgttgtgaggcaggccacacgaactgggggagccccaaacgtgtctgatgcatacactatcaatggtcaacctggtgatctttacaagtgctccagcaaagacaccaccattgtcccaatacatgccggcgagaccaaccttcttcgtgtcatcaatgctgcactcaatcaacctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttggagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtgcatggcttttgcattgtcacttggacgttcacattggatggggtttggctacggtgttgttggtggagaatggagttgggaagttgcaatccatagagcctcctcctgtggatcttcctctttgt

SEQ ID NO: 385 - DNA, 372 bp, Glycine max
atgtctcagggaagaggcagtgcatctttgcctattgtggtcactgtggtttcactactgtgccttttggaacgtgctaacgcagcaacttactccgttggaggacctgggggatggaccttcaacactaatgcttggcccaatggaaaaagattcagagctggtgatatcctaatcttcaactatgactcaacgacccacaatgtggttgctgtggacagaagtggatacaacagctgcaagacaccagggggtgctaaagtgttcagttcagggaaggaccaaatcaaactagcaagagggcagaactacttcatatgtaactaccctggtcactgcgaatctgggatgaaagttgccattaatgcgctg

SEQ ID NO: 386 - DNA, 1350 bp, artificial sequence
  synthetic construct
atggttacttgtcctggttccataataatccatttcttcctcttctcagcgcctctcctttcagtcttatggtcttgttcatcagtttctgcattgaaaccccgtgccttcatcctccccatcgagaaagacccaaccacccttcagtactcaacttccatcgacatgggtacccctccactcacactagatctagtcatcgacatcagagaacgcttcctatggttcgagtgcggcaacgactacaactcctcaacctactaccctgtccgatgtgggactaagaaatgcaagaaagccaagggcacggcttgcattacatgcaccaaccaccctcttaaaacaggatgcaccaacaacacgtgtggtgtagatccattcaaccccttcggcgagttcttcgtgagtggagacgtgggtgaagacatcctaagtagcttacatagcacctcaggtgcacgagcaccttccactttgcacgtgccacgcttcgtctctacatgtgtgtacccagataaatttggggttgagggctttctccagggcctggctaagggcaagaaaggagttttaggccttgcaaggactgctatttccttaccaacacaactcgcagccaaatacaaccttgaacctaagtttgcactttgtttaccttcaacttcaaagtataataagcttggtgatctctttgttggtggtgggccttactatttgccacctcatgatgcttccaaatttctttcctacactccaattctcaccaacccccaaagcacaggtccaatctttgatgctgatccttccagcgagtactttattgacgtgaagtcaatcaagcttgacggtaaaattgttaacgttaacacctccctgctttctattgacagacagggaaatgggggctgcaaacttagtaccgtagttccttacaccaaattccacacttcaatataccagccacttgtgaatgactttgtaaagcaagcagcgcttaggaaaataaagagagtgacttcggtggcaccatttggggcgtgctttgattcaagaaccattggcaagaccgttactggacccaatgtgccgacaattgatctggttctcaaggggggagttcaatggagaatctatggtgccaattcaatggtcaaggtttctaagaatgtgctgtgccttggatttgtggatggaggtttggagccaggaagtcccattgcaacttcgattgtgattggtgggtatcagatggaggacaatcttttggagtttgatcttgtttcctcaaaacttggttttagctcctcccttttactccacatggctagctgttcccacttcagacttgtt

SEQ ID NO: 387 - DNA, 1350 bp, artificial sequence
  synthetic construct
atggttacttgtcctggttccataataatccatttcttcctcttctcagcgcctctcctttcagtcttatggtcttgttcatcagtttctgcattgaaaccccgtgccttcatcctccccatcgagaaagacccaaccacccttcagtactcaacttccatcgacatgggtacccctccactcacactagatctagtcatcgacatcagagaacgcttcctatggttcgagtgcggcaacgactacaactcctcaacctactaccctgtccgatgtgggactaagaaatgcaagaaagccaagggcacggcttgcattacatgcaccaaccaccctcttaaaacaggatgcaccaacaacacgtgtggtgtagatccattcaaccccttcggcgagttcttcgtgagtggagacgtgggtgaagacatcttgagtagcttacatagcacctcaggtgcacgagcaccttccactttgcacgtgccacgcttcgtctctacatgtgtgtacccagataaatttggggttgagggctttctccagggcctggctaagggcaagaaaggagttttaggccttgcaaggactgctatttccttaccaacacaactcgcagccaaatacaaccttgaacctaagtttgcactttgtttaccttcaacttcaaagtataataagcttggtgatctctttgttggtggtgggccttactatttgccacctcatgatgcttccaaatttctttcctacactccaattctcaccaacccccaaagcacaggtccaatctttgatgctgatccttccagcgagtactttattgacgtgaagtcaatcaagcttgacggtaaaattgttaacgttaacacctccctgctttctattgacagacagggaaatgggggctgcaaacttagtaccgtagttccttacaccaaattccacacttcaatataccagccacttgtgaatgactttgtaaagcaagcagcgcttaggaaaataaagagagtgacttcggtggcaccatttggggcgtgctttgattcaagaaccattggcaagaccgttactggacccaatgtgccgacaattgatctggttctcaaggggggagttcaatggagaatctatggtgccaattcaatggtcaaggtttctaagaatgtgctgtgccttggatttgtggatggaggtttggagccaggaagtcccattgcaacttcgattgtgattggtgggtatcagatggaggacaatcttttggagtttgatcttgtttcctcaaaacttggttttagctcctcccttttactccacatggctagctgttcccacttcagacttgtt

SEQ ID NO: 388 - DNA, 1350 bp, artificial sequence; identical to SEQ ID NO: 387
  synthetic construct

SEQ ID NO: 389 - DNA, 1350 bp, artificial sequence; identical to SEQ ID NO: 387
  synthetic construct

SEQ ID NO: 390 - DNA, 1350 bp, artificial sequence; identical to SEQ ID NO: 387
  synthetic construct

SEQ ID NO: 391 - DNA, 1350 bp, artificial sequence; identical to SEQ ID NO: 387
  synthetic construct

SEQ ID NO: 392 - DNA, 1350 bp, artificial sequence
  synthetic construct
atggttacttgtcctggttccataataatccatttcttcctcttctcagcgcctctcctttcagtcttatggtcttgttcatcagtttctgcattgaaaccccgtgccttcatcctccccatcgagaaagacccaaccacccttcagtactcaacttccatcgacatgggtacccctccactcacactagatctagtcatcgacatcagagaacgcttcctatggttcgagtgcggcaacgactacaactcctcaacctactaccctgtccgatgtgggactaagaaatgcaagaaagccaagggcacggcttgcattacatgcaccaaccaccctcttaaaacaggatgcaccaacaacacgtgtggtgtagatccattcaaccccttcggcgagttcttcgtgagtggagacgtgggtgaagacatcctatccagcttacatagcacctcaggtgcacgagcaccttccactttgcacgtgccacgcttcgtctctacatgtgtgtacccagataaatttggggttgagggctttctccagggcctggctaagggcaagaaaggagttttaggccttgcaaggactgctatttccttaccaacacaactcgcagccaaatacaaccttgaacctaagtttgcactttgtttaccttcaacttcaaagtataataagcttggtgatctctttgttggtggtgggccttactatttgccacctcatgatgcttccaaatttctttcctacactccaattctcaccaacccccaaagcacaggtccaatctttgatgctgatccttccagcgagtactttattgacgtgaagtcaatcaagcttgacggtaaaattgttaacgttaacacctccctgctttctattgacagacagggaaatgggggctgcaaacttagtaccgtagttccttacaccaaattccacacttcaatataccagccacttgtgaatgactttgtaaagcaagcagcgcttaggaaaataaagagagtgacttcggtggcaccatttggggcgtgctttgattcaagaaccattggcaagaccgttactggacccaatgtgccgacaattgatctggttctcaaggggggagttcaatggagaatctatggtgccaattcaatggtcaaggtttctaagaatgtgctgtgccttggatttgtggatggaggtttggagccaggaagtcccattgcaacttcgattgtgattggtgggtatcagatggaggacaatcttttggagtttgatcttgtttcctcaaaacttggttttagctcctcccttttactccacatggctagctgttcccacttcagacttgtt

SEQ ID NO: 393 - DNA, 1350 bp, artificial sequence; identical to SEQ ID NO: 392
  synthetic construct

SEQ ID NO: 394 - DNA, 1350 bp, artificial sequence; identical to SEQ ID NO: 392
  synthetic construct

SEQ ID NO: 395 - DNA, 1350 bp, artificial sequence; identical to SEQ ID NO: 392
  synthetic construct

SEQ ID NO: 396 - DNA, 1767 bp, artificial sequence
  synthetic construct
atggagcctgccaaaaccattcacaacaatgtcaaatactcccccatcttcttagccatctttgttctgatcttagcttcagcattgtcttcagcaaatgccaaaattcacgagcacgagtttgttgttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggcacggtgttaggcaaatgagaacagggtgggcagatggaccagaatttgtgacttcagtgcccgattgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcgcgagacacccattcttcttggggaatggtgggacgcaaaccctattgatgttgtgaggcaggccacacgaactgggggagccccaaacgtgtctgatgcatacactatcaatggtcaacctggtgatctttacaagtgctccagcaaagacaccaccattgtcccaatacatgccggcgagaccaaccttcttcgtgtaataaacgcagccttaaaccagcctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttggagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtgcatggcttttgcattgtcacttggacgttcacattggatggggtttggctacggtgttgttggtggagaatggagttgggaagttgcaatccatagagcctcctcctgtggatcttcctctttgt

SEQ ID NO: 397 - DNA, 1767 bp, artificial sequence
  synthetic construct
atggagcctgccaaaaccattcacaacaatgtcaaatactcccccatcttcttagccatctttgttctgatcttagcttcagcattgtcttcagcaaatgccaaaattcacgagcacgagtttgttgttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggcacggtgttaggcaaatgagaacagggtgggcagatggaccagaatttgtgacttcagtgcccgattgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcgcgagacacccattcttcttggggaatggtgggacgcaaaccctattgatgttgtgaggcaggccacacgaactgggggagccccaaacgtgtctgatgcatacactatcaatggtcaacctggtgatctttacaagtgctccagcaaagacaccaccattgtcccaatacatgccggcgagaccaaccttcttcgtgtcataaacgcagccttaaaccagcctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttggagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtgcatggcttttgcattgtcacttggacgttcacattggatggggtttggctacggtgttgttggtggagaatggagttgggaagttgcaatccatagagcctcctcctgtggatcttcctctttgt

SEQ ID NO: 398 - DNA, 1767 bp, artificial sequence; identical to SEQ ID NO: 397
  synthetic construct

SEQ ID NO: 399 - DNA, 1767 bp, artificial sequence; identical to SEQ ID NO: 397
  synthetic construct

SEQ ID NO: 400 - DNA, 1767 bp, artificial sequence
  synthetic construct
atggagcctgccaaaaccattcacaacaatgtcaaatactcccccatcttcttagccatctttgttctgatcttagcttcagcattgtcttcagcaaatgccaaaattcacgagcacgagtttgttgttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggcacggtgttaggcaaatgagaacagggtgggcagatggaccagaatttgtgacttcagtgcccgattgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcgcgagacacccattcttcttggggaatggtgggacgcaaaccctattgatgttgtgaggcaggccacacgaactgggggagccccaaacgtgtctgatgcatacactatcaatggtcaacctggtgatctttacaagtgctccagcaaagacaccaccattgtcccaatacatgccggcgagaccaaccttcttcgtgtaatcaacgcagccttaaaccagcctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttggagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtgcatggcttttgcattgtcacttggacgttcacattggatggggtttggctacggtgttgttggtggagaatggagttgggaagttgcaatccatagagcctcctcctgtggatcttcctctttgt

SEQ ID NO: 401 - DNA, 1767 bp, artificial sequence; identical to SEQ ID NO: 400
  synthetic construct

SEQ ID NO: 402 - DNA, 1767 bp, artificial sequence; identical to SEQ ID NO: 400
  synthetic construct

SEQ ID NO: 403 - DNA, 1767 bp, artificial sequence
  synthetic construct
atggagcctgccaaaaccattcacaacaatgtcaaatactcccccatcttcttagccatctttgttctgatcttagcttcagcattgtcttcagcaaatgccaaaattcacgagcacgagtttgttgttgaagcaactccagtgaagaggctgtgcaaaacccacaacagcatcaccgtgaatggacaatacccgggcccaacgttggaaatcaacaatggagacactttggtcgtcaaagtcactaacaaagctcgttacaatgtgaccattcattggcacggtgttaggcaaatgagaacagggtgggcagatggaccagaatttgtgacttcagtgcccgattgtccaggaggaagttacacctaccgttttaccgttcaaggacaagaaggcacactttggtggcacgctcatagctcatggttaagggccaccgtttacggtgctttaatcattcgtcctagggaaggagaaccctaccctttccccaagcctaagcgcgagacacccattcttcttggggaatggtgggacgcaaaccctattgatgttgtgaggcaggccacacgaactgggggagccccaaacgtgtctgatgcatacactatcaatggtcaacctggtgatctttacaagtgctccagcaaagacaccaccattgtcccaatacatgccggcgagaccaaccttcttcgtgtaataaatgcagccttaaatcagcctctcttcttcaccgtcgcaaaccacaaactcacagtggttggtgccgacgcctcctacctcaaacccttcaccaccaaagtcctcatactgggccccgggcaaaccaccgacgtcttaatcaccggcgaccagccaccttcccgctactacatggcggcgcgtgcgtaccaatccgcccaaaacgctgccttcgacaacaccactacaaccgccatactcgaatacaaatcaccgaatcaccacaataagcattctcaccatcgtgccaaaggagtaaagaacaaaaccaaacctataatgcctccactccctgcttacaacgacacaaacgcagtcacttccttcagcaaaagcttcagaagccctagaaaagttgaagtacccactgaaattgaccagagcctcttcttcactgtgggtttaggtatcaagaagtgccccaaaaacttcggaccaaagaggtgtcagggacccattaatgggacgaggttcactgcgagcatgaacaacgtgtctttcgttctcccgaacaacgtgtccatcttgcaggctcaccacctcggaatccctggagtgttcaccactgattttccggggaagccgccggtgaagtttgattacaccggcaatgtgagccgttcgctgtggcaacctgttcccgggacaaaggcacacaagttggagtttgggtcgagggtgcagattgtgttgcaggatactagcattgtcactcctgagaaccaccctatccatcttcatgggtacgatttctacattgttgcagagggtttcgggaacttcgacccaaagaaagatacggcgaaattcaaccttgttgatccacctttgagaaacacagtggctgtgcctgtaaatggatgggcagttattcgatttgtggctgataacccaggtgcatggcttttgcattgtcacttggacgttcacattggatggggtttggctacggtgttgttggtggagaatggagttgggaagttgcaatccatagagcctcctcctgtggatcttcctctttgt

SEQ ID NO: 404 - DNA, 1767 bp, artificial sequence; identical to SEQ ID NO: 403
  synthetic construct

SEQ ID NO: 405 - DNA, 1767 bp, artificial sequence; identical to SEQ ID NO: 403
  synthetic construct

SEQ ID NO: 406 - DNA, 372 bp, artificial sequence
  synthetic construct
atgagccaaggccgcggatcagccagcttgcctattgtggtcactgtggtttcactactgtgccttttggaacgtgctaacgcagcaacttactccgttggaggacctgggggatggaccttcaacactaatgcttggcccaatggaaaaagattcagagctggtgatatcctaatcttcaactatgactcaacgacccacaatgtggttgctgtggacagaagtggatacaacagctgcaagacaccagggggtgctaaagtgttcagttcagggaaggaccaaatcaaactagcaagagggcagaactacttcatatgtaactaccctggtcactgcgaatctgggatgaaagttgccattaatgcgctg

SEQ ID NO: 407 - DNA, 372 bp, artificial sequence
  synthetic construct
atgagccagggccgcggatcagccagcttgcctattgtggtcactgtggtttcactactgtgccttttggaacgtgctaacgcagcaacttactccgttggaggacctgggggatggaccttcaacactaatgcttggcccaatggaaaaagattcagagctggtgatatcctaatcttcaactatgactcaacgacccacaatgtggttgctgtggacagaagtggatacaacagctgcaagacaccagggggtgctaaagtgttcagttcagggaaggaccaaatcaaactagcaagagggcagaactacttcatatgtaactaccctggtcactgcgaatctgggatgaaagttgccattaatgcgctg

SEQ ID NO: 408 - DNA, 372 bp, artificial sequence; identical to SEQ ID NO: 407
  synthetic construct

SEQ ID NO: 409 - DNA, 372 bp, artificial sequence; identical to SEQ ID NO: 407
  synthetic construct

SEQ ID NO: 410 - DNA, 372 bp, artificial sequence; identical to SEQ ID NO: 407
  synthetic construct

SEQ ID NO: 411 - DNA, 24 bp, artificial sequence
  synthetic construct
catcaacgctgctacactcaataa

SEQ ID NO: 412 - DNA, 24 bp, artificial sequence
  synthetic construct
gccagggaagactagtcagtgcag

SEQ ID NO: 413 - DNA, 24 bp, artificial sequence
  synthetic construct
tccttggcaacctagacacctcca

SEQ ID NO: 414 - DNA, 24 bp, artificial sequence
  synthetic construct
tgtcctcgctgctacactcaacaa

SEQ ID NO: 415 - DNA, 24 bp, artificial sequence
  synthetic construct
ctcggggaagactaggcagtgcag

SEQ ID NO: 416 - DNA, 24 bp, artificial sequence
  synthetic construct
accgtagcagcctagacacctccc

SEQ ID NO: 417 - DNA, 1652 bp, artificial sequence
  synthetic construct
ggggagcaatacaagatcttaatttctttcacaaatcacaatggttacttgtcctggttccataataatccatttcttcctcttctcagcgcctctcctttcagtcttatggtcttgttcatcagtttctgcattgaaaccccgtgccttcatcctccccatcgagaaagacccaaccacccttcagtactcaacttccatcgacatgggtacccctccactcacactagatctagtcatcgacatcagagaacgcttcctatggttcgagtgcggcaacgactacaactcctcaacctactaccctgtccgatgtgggactaagaaatgcaagaaagccaagggcacggcttgcattacatgcaccaaccaccctcttaaaacaggatgcaccaacaacacgtgtggtgtagatccattcaaccccttcggcgagttcttcgtgagtggagacgtgggtgaagacatcttgtcctcgctgctacactcaacaagcggtgcacgagcaccttccactttgcacgtgccacgcttcgtttctacatgtgtgtacccagataaatttgggggttgagggctttctccagggcctggctaaagggcaagaaaggagttttaggccttgcaaggactgctatttccttaccaacacaactcgcagccaaatacaaccttgaacctaagtttgcactttgtttaccttcaacttcaaagtataataagcttggtgatctctttgttggtggtgggccttactatttgccacctcatgatgcttccaaatttctttcctacactccaattctcaccaacccccaaagcacaggtccaatctttgatgctgatccttccagcgagtactttattgacgtgaagtcaatcaagcttgacggtaaaattgttaacgttaacacctccctgctttctattgacagacagggaaatgggggctgcaaacttagtaccgtagttccttacaccaaattccacacttcaatataccagccacttgtgaatgactttgtaaagcaagcagcgcttaggaaaataaagagagtgacttcggtggcaccatttggggcgtgctttgattcaagaaccattggcaagaccgttactggacccaatgtgccgacaattgatctggttctcaaggggggagttcaatggagaatctatggtgccaattcaatggtcaaggtttctaagaatgtgctgtgccttggatttgtggatggaggtttggagccaggaagtcccattgcaacttcgattgtgattggtgggtatcagatggaggacaatcttttggagtttgatcttgtttcctcaaaacttggttttagctcctcccttttactccacatggctagctgttcccacttcagacttgtttgacttttcactttcgatcatttcagcaaagtttggttcatttggtgatgactgatgaataaattttatttgccattgtaatcgtatggaatatatgctcatttcactctcggtgtgttgggataaaccaaacattctaagtttgttgtgtatttatttcattataagcactagtcagtaagcattttcatttttgttccatcctacttttatatttctcattgtttacccatactataataaatcaataataaagg

SEQ ID NO: 418 - DNA, 328 bp, artificial sequence
  synthetic construct
ggctcggggaagactaggcagtgcagtggttctactactttgcttcttgctgcttcactctcagatggctcgtgctgccacctacacagctggagattctgggggttggacctttaacactgttgcctggcccaaaggaaagctctttcgggctggtgacacacttgcttttaattatagccctgggactcacattgtggtggccgtgaacaaggctggatatgatagctgcaacactccaagaggagccaaagtgtttaagtcagggacggatcagatcagacttgccatgggacacaactactttcatagcccttatgttggtcat

SEQ ID NO: 419 - DNA, 387 bp, artificial sequence
  synthetic construct
cggccggggagggagaatttaggttttttagggttttactaccgtagcagcctagacacctccctcccgcagtagccatggttctcaaaactgaactatgccgattcagtggtgccaagatctaccccggaaagggcatcagatttgttcgtggtgattctcaggttttcctgtttgccaactcaaaatgtaagcggtatttccacaaccgcctgaagccctcaaagctcacctggactgctatgtacagaaagcaacacaaaaaggacattgctcaagaagctgtgaagaagaggagacgcgctgccaaaaagccttactctaggtccattggtggtgccactcctgaagttatccagaaaaagagagctgagaagagatcaagaa